(19) United States
(12) Patent Application Publication
(10) Pub. No.: US 2024/0124910 A1
(43) Pub. Date: Apr. 18, 2024

(54) RIBOSOME-MEDIATED POLYMERIZATION OF NOVEL CHEMISTRIES

(71) Applicants: Northwestern University, Evanston, IL (US); BOARD OF REGENTS, THE UNIVERSITY OF TEXAS SYSTEM, Austin, TX (US)

(72) Inventors: Michael Christopher Jewett, Evanston, IL (US); Joongoo Lee, Evanston, IL (US); Eric V. Anslyn, Austin, TX (US); Jaime Coronado, Austin, TX (US); Jongdoo Lim, Austin, TX (US)

(21) Appl. No.: 18/264,025

(22) PCT Filed: Feb. 2, 2022
(86) PCT No.: PCT/US2022/014889

§ 371 Date: Aug. 2, 2023

Related U.S. Application Data
(60) Provisional application No. 63/144,814, filed on Feb. 2, 2021.

(51) Int. Cl. C12P 19/34; C12P 21/02

(52) U.S. Cl. CPC C12P 19/34; C12P 21/02

(57) ABSTRACT

Disclosed are methods, systems, components, and compositions for synthesis of sequence defined polymers. The methods, systems, components, and compositions may be utilized for incorporating novel substrates that include non-standard amino acid monomers and non-amino acid monomers into sequence defined polymers. As disclosed herein, the novel substrates may be utilized for acylation of tRNA via flexizyme catalyzed reactions. The tRNAs thus acylated with the novel substrates may be utilized in synthesis platforms for incorporating the novel substrates into a sequence defined polymer.

### CROSS-REFERENCE TO RELATED APPLICATIONS

This application claims the benefit of U.S. Application No. 63/144,814 filed on Feb. 2, 2021, the entire content of which is incorporated herein by reference.

### STATEMENT REGARDING FEDERALLY SPONSORED RESEARCH OR DEVELOPMENT

This invention was made with government support under W911NF-16-1-0372 awarded by the Army Research Office. The government has certain rights in the invention.

### SEQUENCE LISTING

A Sequence Listing accompanies this application and is submitted as an ASCII text file of the sequence listing named “702581_02084_ST25.txt” which is 7,968 bytes in size and was created on Feb. 1, 2022. The sequence listing is electronically submitted via EFS-Web with the application and is incorporated herein by reference in its entirety.

### FIELD

The field of the invention relates to components and methods for preparing novel chemical moieties and sequence defined polymers comprising the chemical moieties. In particular, the field of the inventions related to components and methods for the synthesis of chemical moieties comprising optionally substituted pyrazolone groups, optionally substituted pyridazinone groups, and optionally substituted diazepinone groups, and sequence defined polymers into which the optionally substituted pyrazolone groups, the optionally substituted pyridazinone groups, and the optionally substituted diazepinone groups have been incorporated. The components include and the methods utilize a variety of oxo-acid precursors and hydrazineyl acid precursors and cell-free protein synthesis platforms in order to prepare the disclosed optionally substituted pyrazolone groups, optionally substituted pyridazinone groups, and optionally substituted diazepinone groups, and sequence defined polymers into which the optionally substituted pyrazolone groups, the optionally substituted pyridazinone groups, and the optionally substituted diazepinone groups have been incorporated.

### BACKGROUND

The flexizyme (Fx) enzymes (eFx, dFx, and aFx) are RNA enzyme developed through directed evolution and sequence optimization. The Fx is capable of aminoacylating the 3′-OH of an arbitrary tRNA with activated esters. Thus, the (Fx) system enables reprogramming of the genetic code by reassigning the codons that are generally assigned to natural amino acids to non-natural residues. Accordingly, non-natural polypeptides or polymers can be made by mRNA-directed synthesis. Current studies have reported more than 200 non-canonical substrates can be charged into tRNA and incorporated into a peptide by the Fx approach, and multiple strategies have been devised to synthesize tRNAs charged with non-canonical amino acid. However, there still exist limitations and gaps in the range of substrates.

The Fx system has seen widespread success over the last decade in which a wide range of chemical substrates (α-amino acids, β-amino acids, γ-amino acids, D-amino acids, nonstandard amino acids, N-protected (alkylated) amino acids, fluorescent amino acids, and hydroxy acids, aromatic, aliphatic, malonyl, and oligomeric amino acids) have been incorporated into a ribosomal peptide chain through mis-acylated tRNAs. The system has produced different types of polymers such as polyamides, polyesters, polythioesters, polythioamides.

However, the chemical bond synthesized so far other than an amide-(peptide) bond has been confined to ester and thioester by the use of hydroxy and thioacids because the translational machinery has been evolutionarily optimized to form amide using the canonical 20 amino acid building blocks.

Here, the inventors disclose new covalent chemical bonds by using rationally designed non-canonical monomer substrates that are charged to tRNA and form a new chemical bond by the ribosome-mediated protein translation process in a cell-free platform. In particular, the inventors investigated the creation of heterocyclic chemical bonds between oxo-acid substrates (e.g., β-keto esters, γ-keto esters, and δ-keto esters) and hydrazineyl acid substrates and demonstrate that the ribosome can be used as a chemical machine to conjugate the oxo-acid substrates and hydrazineyl substrates to form optionally substituted pyrazolone groups, optionally substituted pyridazinone groups, and optionally substituted diazepinone groups and build a sequence-defined polymers based on the information read from mRNA.

The inventors demonstrate that the disclosed substrates and ribosome-mediated polymerization thereof produce polymers having structures that are unobtainable by the post-translational modification machinery or other naturally occurring synthesis methods. Furthermore, polymers produced by the disclosed substrates and ribosome-mediated polymerization thereof have novel functionality, which may be useful in creating next-generation based-commodities such as polymers and therapeutics that need to be precisely designed for high-tech science and personalized drugs. The disclosed technology may be utilized as a foundational resource for chemists, biochemists, and molecular biologists as well as protein engineers to prepare novel chemical moieties and polymers comprising the novel chemical moieties. Finally, the disclosed technology may be utilized to prepare further, novel chemical substrate variants for the synthesis of various peptides, including precursors for therapeutic medicines and macrocyclic materials. As such, the disclosed technology has advantages for fundamental and synthetic/engineering biology.

### SUMMARY

Disclosed herein are components and methods that include or utilize novel chemical moieties and sequence defined polymers into which the novel chemical moieties have been incorporated. The novel chemical moieties may be prepared from substrates which are utilized as monomers for preparing sequence defined polymers. As disclosed herein, the substrates may be utilized for acylation of tRNA via flexizyme catalyzed reactions. The tRNAs thus acylated with the novel substrates may be utilized in synthesis platforms for incorporating the novel substrates into a sequence defined polymer via translation, and the novel substrates thus incorporated further may be conjugated in order to form the novel chemical moieties.

The components disclosed herein include chemical substrates and acylated tRNA molecules comprising the chemical substrates, where the chemical substrates may be utilized as precursors or monomers that may be incorporated into a sequence defined polymer. As such, tRNAs may be acylated with the disclosed chemical substrates in order to form the disclosed acylated tRNA molecules.

In some embodiments, the chemical substrate has a Formula I or II:

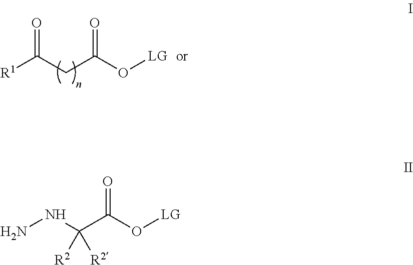

wherein:
n is 1-10 (or 1-9, 1-8, 1-7, 1-6, 1-5, 1-4, or 1-3);
R¹ is hydrogen, alkyl (e.g., methyl, ethyl), aryl (e.g., phenyl) which optionally is substituted at one or more positions with alkyl or alkylthio (e.g., 4-methylthio-phenyl),
R² is hydrogen, alkyl (e.g., methyl, isopropyl), alkylaryl (e.g., benzyl) which optionally is substituted at one or more positions with hydroxyl (e.g., 3,4-dihydroxy-benzyl), or R2 is the side chain of an amino acid (e.g., a side chain of an amino acid selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine;
R^{2′} is hydrogen or alkyl (e.g., methyl); and
LG is a leaving group that is removed when the chemical moiety is utilized to acylate a tRNA molecule (e.g., when the chemical moiety is utilized to acylate a tRNA molecule at the C3 hydroxyl group) and form an acylated tRNA having a Formula I(a) or II(a):

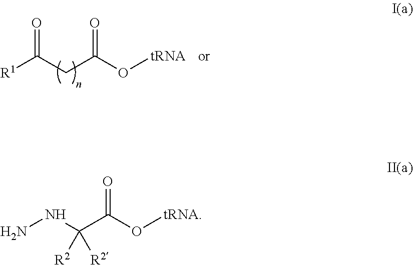

In the disclosed methods, a chemical substrate comprising a keto ester group and a chemical substrate comprising a hydrazine group may be conjugated to form a heterocyclic compound that may provide a linkage in a polymer. In some embodiments of the disclosed methods, the chemical substrates may be conjugated to form novel optionally substituted pyrazolone, optionally substituted pyridazinones, and optionally substituted diazepinones. In some embodiments, the chemical substrates (e.g., as part of acylated tRNAs) may be conjugated via a ribosome to form optionally substituted pyrazolones, optionally substituted pyridazinones, and optionally substituted diazepinones. The optionally substituted pyrazolones, optionally substituted pyridazinones, and optionally substituted diazepinones thus formed may provide a novel linkage in the backbone of a polymer. In some embodiments, the heterocyclic compound thus formed may provide a linkage having a structure:

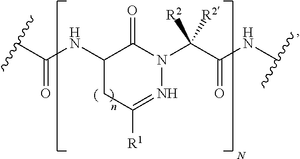

wherein n is 0-10, R¹, R², and R^{2′} are as defined above for Formula I, I(a), II, and II(a), and N is 1-100 or higher.

### BRIEF DESCRIPTION OF THE FIGURES

FIG. 1. Provides a schematic of the flexizyme (Fx) system for Fx mediate acylation of tRNAs with oxo-acid variants (e.g., β-, γ-, and δ-keto esters) and hydrazineyl acid variants, followed by ribosome-mediated polymerization in vitro. Depending on the number of carbons in the oxo-acid variant, the resulting compounds comprise a bond including either a 5, 6, or 7-membered ring, formed by the ribosome. Flexizyme leaving groups include, but are not limited to Cyanomethylester (CME), dinitrobenzylester (DNB), chlorobenzylthioester (CBT), and amino-derivatized benzylthioester (ABT). Methods to link the leaving group to the substrate are well known in the art, see e.g., Nature Communications volume 10, Article number: 5097 (2019); Nature Communications volume 11, Article number: 4304 (2020); Nature Protocols volume 6, pages 779-790(2011), incorporated herein by reference in their entireties.

FIG. 2A-B. Provides structures of exemplary molecules that were charged to tRNA, and then incorporated into a polymer via a cell-free synthesis system using wild-type ribosomes. (A) substrate structures; (B) acylation yield (% of Fx-charged tRNA with substrates) first set of bars; and N-terminus incorporation efficiency (second set of bars). Acylation reactions were performed as follows. Acylation of microhelix (small tRNA mimic): 1 μL of 0.5 M HEPES (pH 7.5) or bicine (pH 8.8), 1 μL of 10 μM microhelix, and 3 μL of nuclease-free water were mixed in a PCR tube with 1 μL of 10 μM eFx, dFx, and aFx, respectively. The mixture was heated for 2 min at 95° C. and cooled down to room temperature over 5 min. 2 μL of 300 mM MgCl₂ was added to the cooled mixture and incubated for 5 min at room temperature. Followed by the incubation of the reaction mixture on ice for 2 min, 2 μL of 25 mM activated ester substrate in DMSO was added to the reaction mixture. The reaction mixture was further incubated for 12 h on ice in cold room. Microhelix was purchased from IDT. (Sequence: 5′-GGCUCUGUUCGCAGAGCCGCCA-5′ (SEQ ID NO: 1). Acylation of tRNA. 2 μL of 0.5 M HEPES (pH 7.5) or 0.5 M bicine (pH 8.8), 2 μL of 250 μM tRNA, 2 μL of 250 μM of a Fx selected on the microhelix experiment and 6 μL of nuclease-free water were mixed in a PCR tube. The mixture was heated for 2 min at 95° C. and cooled down to room temperature over 5 min. 4 μL of 300 mM MgCl₂ was added to the cooled mixture and incubated for 5 min at room temperature. Followed by the incubation of the reaction mixture on ice for 2 min, 4 μL of 25 mM activated ester substrate in DMSO was added to the reaction mixture. The reaction mixture was further incubated for the optimal time determined on the microhelix experiment on ice in cold room. The tRNAs were ethanol-precipitated to wash unreacted substrates before being subjected a cell-free protein synthesis reaction. The acylated tRNAs were then subject to a cell-free protein synthesis system, to test the ability of wild-type ribosomes in the system to add the substrate to N-terminus of a 10-mer peptide (the length of the final product can vary depending on the DNA template design). The polymerization reaction was carried out as follows. A reporter peptide, a T7 promoter-controlled DNA template (pJL1_StrepII_1, encoded as ATGACCTGGTCTCATCCGCAGTTCGAAAAATAG (SEQ ID NO: 2)) was designed to encode a streptavidin (Strep) tag and additional Met and Thr codons (XYWHSPQFEK (SEQ ID NO: 3) (strep-tag), where X and Y indicate the position of the non-canonical chemical substrates). The translation initiation codon AUG and the Thr codon (ACC) were used for two consecutive incorporation, X and Y. Peptide synthesis was performed using only the 8 amino acids (100 μM) that decode the purification tag in the absence of the other 12 amino acids to prevent corresponding endogenous tRNAs from being aminoacylated and used in translation. The PURExpress® Δ (aa, tRNA) or PURExpress® Δ (aa, tRNA, ribosome) kit was used for ring formation reaction and the reaction mixtures were incubated at 37° C. for 3 h. The synthesized peptides were then purified using Strep-Tactin®-coated magnetic beads (IBA), denatured with 0.1% SDS, and characterized by MALDI-TOF mass spectroscopy. As shown in the bar graph (B), acylation yield and the N-terminus incorporation efficiency are consistent. The N-terminus incorporation is highly dependent on the acylation yields.

FIG. 3A-N. Provides a comparison of the performance of wild type and engineered ribosomes to form pyridazinone bonds from the following charged-tRNA substrates:

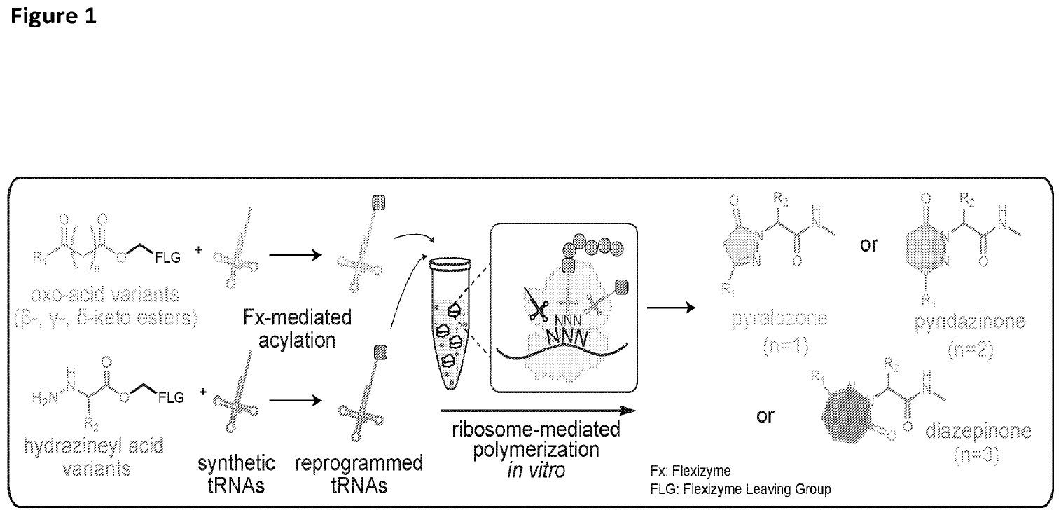

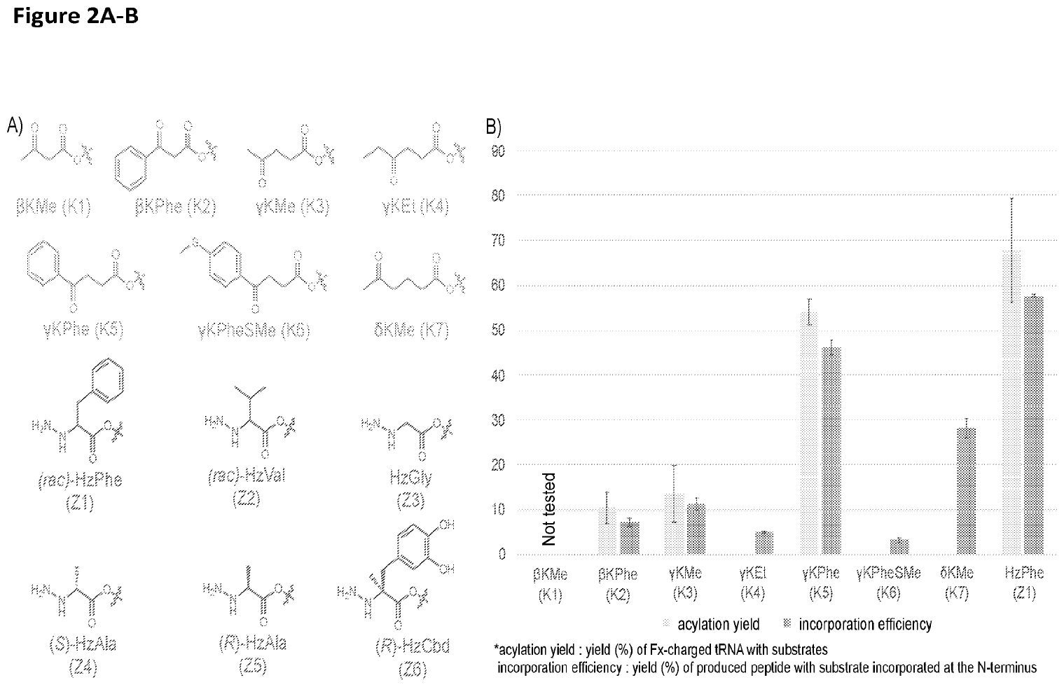

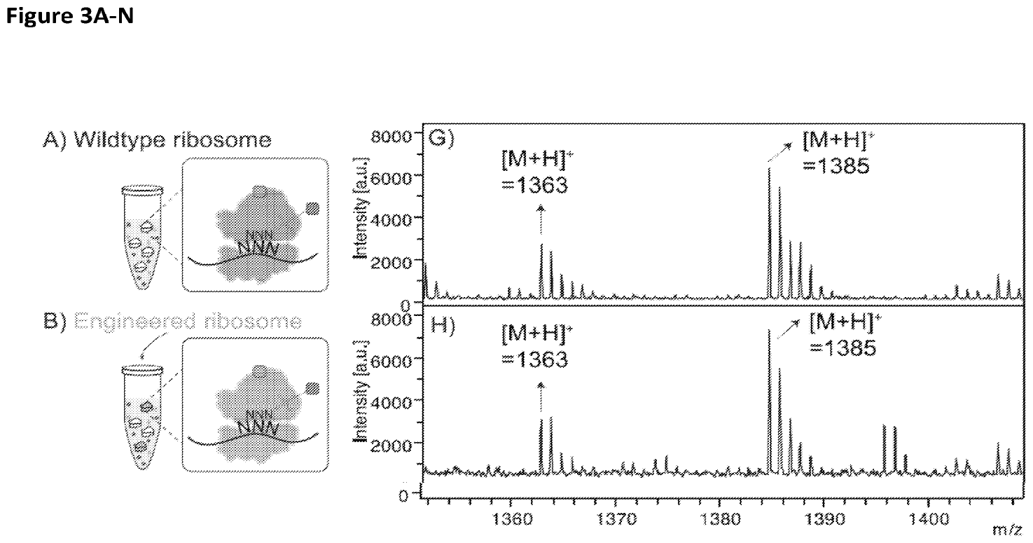

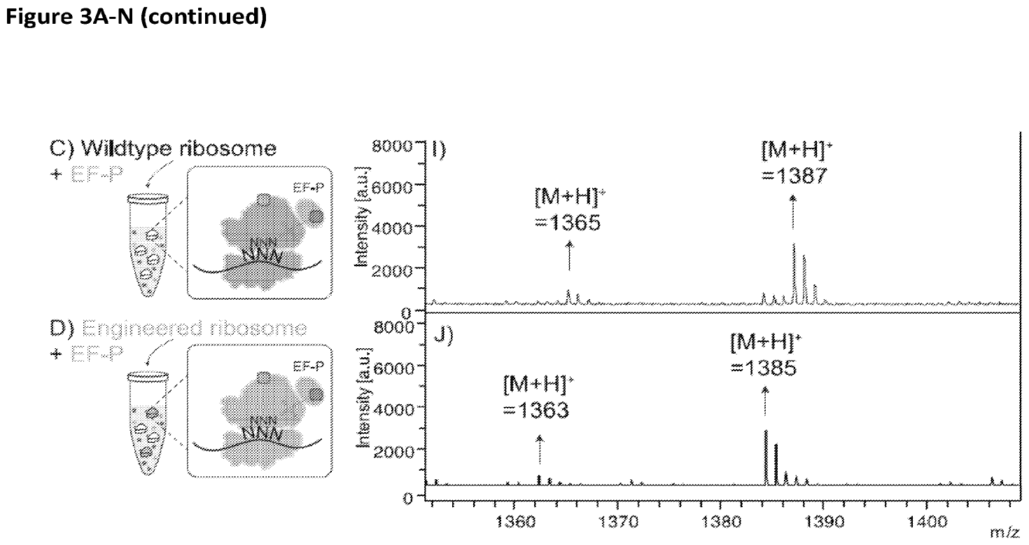

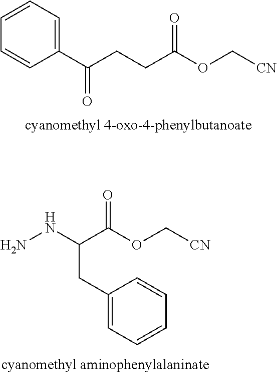 [SMILES: N#CCOC(=O)CCC(=O)c1ccccc1 ; N#CCOC(=O)C(Cc1ccccc1)NN]

Elongation factor (“EF”) EF-P (see e.g., Nat Commun 7, 11657 (2016); Chem. Commun., 2020, 56, 5597-5600, incorporated herein by reference in entirety), EF-Tu mutant (E69D), and EF-Tu mutant (Y771), (see e.g., Nat Commun 7, 11657 (2016); ACS Synth. Biol. 2019, 8, 2, 287-296, incorporated by reference in its entirety). The mutation sites were designed through reconstructing evolutionary adaptive path (REAP) analysis of EF-Tu and its analog SelB. The mutation sites are expected to influence EF-Tu binding ability to aa-tRNA and are located close to the amino acid binding pocket of EF-Tu. The mutated amino acids may or may not interact with the tRNA directly but can still influence binding to EF-Tu. The substrate-promiscuous EF-Tu mutants were identified using in vivo fluorescence assays. The substrate-charged tRNAs were precipitated and added into the PURExpress reaction in the presence of 10 μM (in final) EF-P or EF-Tu. Addition of EF-P and EF-Tu did not significantly increase the yield of the pyridazinone bonds. As shown in A and B, while both the wild-type and engineered ribosomes formed heterocyclic bonds, the engineered ribosomes performed slightly better.

FIG. 4. Provides a table showing exemplary substrate compounds and the resultant product after cell-free ribosome-directed synthesis. The top row provides one exemplary set of substrate compounds, and the far left column provides a second set of exemplary substrates. When charged to a tRNA by Fx and subjected to cell-free protein synthetic conditions, the product produced for each combination, presented at the intersection of the row and column, is shown.

FIG. 5. Shows the production of peptides including an oxo- or hydrazineyl acid substrate. The five different keto esters were charged to separately to fMet (CAU) by Fx separately. HzPhe (Z1) was charged to Pro1E2(GGU). The tRNAs were washed by ethanol-precipitation and added into the PURE system in which the plasmid DNA encoding XYWSHPQFEK (SEQ ID NO: 7) was contained. Keto ester and Z1 is encoded for the position of X and Y, respectively. Five sets of peptide synthesis reactions were performed as described above separately using only the 8 amino acids (100 μM) that decode the purification tag in the absence of the other 12 amino acids. The yield is important because it shows ribosome ability to form non-amide bond formation.

FIG. 6. Provides a schematic for production of sequence defined alternating copolymers. The figure illustrates the consecutive incorporation of oxo- or hydrazineyl acid substrate monomers into a peptide, producing various polymers containing multiple cyclic bonds. Three different plasmids (pJL1-StrepII_AB, pTh1-StrepII_ABA, pTh1-StrepII_ABAB, encoding (MWHSPQFEKSAB (SEQ ID NO: 4), MWHSPQFEKSABA (SEQ ID NO: 5), MWHSPQFEKSABAB (SEQ ID NO: 6), where A and B indicate the position of the K8 and Z1 substrates) were used for C-terminal incorporation. K8 and Z1 were incorporated at the A and B position, respectively using two tRNAs (Pro1E2 (GGU) and GluE2(GAU)) in the PURExpress system.

FIG. 7. Ribosome-catalyzed formation of pyridazinone bonds in vitro. Genetic code reprogramming using the flexizyme system enables the acylation of non-canonical substrates with tRNA. Upon flexizyme-mediated tRNA acylation of keto (orange) and hydrazino (green) activated esters, the programmed keto-tRNA (orange) and hydrazino-tRNA (green) were added to an in vitro transcription and translation platform using purified components and allowed to decode two consecutive codons programmed on an mRNA strand. The translation mixture produced a novel pyridazinone bond (pink). The typical peptide bond (red) is shown on the right.

FIG. 8A-E. Design of γ-keto and hydrazino esters and ribosome-mediated synthesis of pyridazinone bonds. (A) Four γ-keto (orange) and (B) two hydrazino (green) esters were synthesized with an activated leaving group, such as cyanomethyl ester (CME), dinitrobenzyl ester (DNB), and amino-derivatized benzylthio ester (ABT). DNB or ABT were used for the substrates that do not contain an aromatic moiety and the ABT-activated substances were only synthesized when the DNB substrates were found to be water-insoluble (1-CME, 2-CME, 3-DNB, 3-ABT, 4-DNB; 5-CME, 6-DNB, 6-ABT). The substrates were charged to tRNA by the appropriate Fx and introduced to an in vitro translation reaction containing wild-type ribosomes. (C) In vitro translation reactions were carried out with pairs to γ-keto ester substrates in panel A and hydrazino ester substrates in panel B. The ribosome catalyzed the synthesis of eight different pyridazinone rings. The relative percent yield of target oligomer of all species as determined by peak area is shown in matrix-assisted laser desorption/ionization-time of flight (MALDI-TOF) mass spectra (Supplementary Information). Percent yield is based on n=3 reactions. (D-E) MALDI-TOF mass spectra of oligomers polymerized by the ribosome in vitro with a pyridazinone bond because of the polymerization reactions between 1 and 5, and 1 and 6, respectively. The calculated mass of the products in D are [M+H]⁺=1362, [M+Na]⁺=1384 and in E are [M+H]⁺=1286, [M+Na]⁺=1308. See SI for MALDI-TOF mass spectra of the other pyridazinone bonds represented in C. The 1305 and 1327 peaks marked by an asterisk (*) are unidentified. Spectra in D and E are representative of n=3 independent experiments.

FIG. 9A-D. The ribosome is required for pyridazinone formation under in vitro translation conditions. (A) The in vitro polymerization reaction was conducted using the same conditions that produced an N-terminal pyridazinone bond in an oligomer, but without the presence of ribosomes. In the deconvoluted mass spectra, the compounds having a molar mass of 179.0701 and 181.1017 were observed as a single peak at 4.4 and 3.6 min, suggesting that they are 4-oxo-4-phenylbutanoic acid (orange in B) aminophenylalanine (green in C) hydrolyzed from tRNA^{fMet}(CUA) and tRNA^{Pro1E2}(GGU), respectively. (D) No species corresponding to the theoretical mass of OPDP (322.1317) were observed in the reaction mixture. The extracted ion chromatograms were obtained based on theoretical average masses (Supplementary Information). Spectra are representative of n=3 independent experiments.

FIG. 10A-C. Ribosomal synthesis of alternating copolymers with a pyridazinone backbone. (A) We designed an additional amino acid, γKPheA (7), bearing a ketone on its g-carbon of the side-chain, for sequential polymerization of pyridazinones bonds on a biopolymer chain. Compounds 7 and 6 were charged to tRNA^{Pro1E2}(GGU) and tRNA^{GluE2}(GAU) by flexizyme, respectively, and added to an in vitro transcription and translation reaction. The genetic template was designed to consecutively incorporate the monomers in an alternating fashion (ABAB- or ABABAB-type). The resulting peptides-pyridazinone hybrids were purified via the Streptavidin tag (WSHPQFEK (SEQ ID NO: 7)) and characterized by MALDI-TOF mass spectrometry. (B) MALDI mass spectrum of the StrepII-7676 peptide (relative yield: 14.8%) and its molecular structure, calculated mass: [M+H]⁺=1791; [M+Na]⁺=1813 (C) MALDI mass spectrum of the StrepII-767676 (relative yield: 16.9%) peptide and its molecular structure, calculated mass: [M+H]⁺=2034; [M+Na]⁺=2056.

FIG. 11. Acylation of microhelix with substrates 1-7. The Fx-catalyzed acylation reaction using the 7 substrates were monitored at two different pH (7.5 and 8.8) over 48 h with three different flexizymes (eFx, dFx, and aFx). The yield of each reaction was determined by quantifying the relative band intensity of unacylated and acylated microhelix (mihx) on the gel using ImageJ software. Fx: Flexizyme, mihx: microhelix. The red arrows indicate the selected acylation reaction conditions for tRNA acylation with the substrate. 3-DNB and 6-DNB were not charged to mihx, presumably because of poor water-solubility. Substrate structures for 1-7 are shown in FIGS. 8 and 10.

FIG. 12A-E. Control experiments for pyridazinone bond reactions. To confirm that the pyridazinone is only formed when the tRNA^{fMet}(CAU):1 and tRNA^{Pro1E2}(GGU):5 complexes are supplemented into the reaction mixture, we carried out three control experiments under the same condition. (A) Mass spectrum of the peptide in the presence of all 20 natural amino acids and absence of Fx-charged tRNA. (B) Mass spectrum of the peptide produced in the presence of 9 amino acids (T+WHSPQFEK (SEQ ID NO: 20)) and tRNA^{fMet}(CAU):1 complex. (C) Mass spectrum of the peptide produced in the presence of 9 amino acids (M+WHSPQFEK (SEQ ID NO: 20)) and tRNA^{Pro1E2}(GGU):5 complex. (D) Mass spectrum of the peptide in the presence of 8 amino acids (WHSPQFEK (SEQ ID NO: 20)) and tRNA^{fMet}(CAU):1 and tRNA^{Pro1E2}(GGU):5 complexes. (E) The enlarged mass spectrum (red box in D) shows a set of isotopic masses that increase sequentially by 1 Da from the theoretical mass.

FIG. 13A-C. Ribosome-mediated synthesis of pyridazinone derivatives with HzPhe (5). The pyridazinone bonds were derivatized by the use of the other γ-keto esters with HzPhe (5). For this, 2 and 5, 3 and 5, and 4 and 5 were consecutively incorporated into the AUG and ACC codon on an mRNA using tRNA^{fMet}(CAU) and tRNA^{Pro1E2}(GGU), respectively. The theoretical molecular weight and structure are shown in the right panel of each mass spectrum. The observed masses are [M+H]⁺=1409, [M+Na]⁺=1431(A); [M+Na]⁺=1323 (B); [M+H]⁺=1314, [M+Na]⁺=1336 (C).

FIG. 14A-C. Ribosome-mediated synthesis of pyridazinone derivatives with HzAla (6). The pyridazinone bonds were derivatized by the use of the other γ-keto esters with HzAla (6). For this, 2 and 6, 3 and 6, and 4 and 6 were consecutively incorporated into the AUG and ACC codon on an mRNA using tRNA^{fMet}(CAU) and tRNA^{Pro1E2}(GGU), respectively. The theoretical molecular weight and structure are shown in the right panel of of each mass spectrum. The observed masses are [M+H]⁺=1332, [M+Na]⁺=1354, [M−H+Na]⁺=1376 (A); [M+H]⁺=1224, [M+Na]⁺=1246 (B); [M+H]⁺=1238, [M+Na]⁺=1260 (C).

FIG. 15A-B. Two possible mechanisms of pyridazinone bond formation in the ribosome. (A) The β-nitrogen atom of hydrazineyl ester (green) coming into the A-site of the ribosome the carbonyl of the ketone of γ-keto ester (orange) to form an imine followed by removal of water. Next, the α-nitrogen atom attacks the ester bond to tRNA, thereby resulting in the formation of pyridazinone bond. (B) The α-nitrogen atom forms a hydrazone with ketone and then β-nitrogen atom forms a hydrazone, resulting in production of a pyridazinone bond.

FIG. 16A-D. Monitoring the pyridazinone formation reaction. The pyridazinone produced in the reaction of cyanomethyl 4-oxo-4-phenylbutanoate with phenylhydrazine hydrochloride in MeOH/H₂O (3/2: v/v) at 37° C. The reactions were monitored by LC-MS with different substrate concentrations of 40 μM (A), 4 mM (B), and 40 mM (C). The fractions collected at the 7.7 min peak (orange box in panel C) was confirmed to be a 2,6-substituted pyridazinone (D) through ¹H NMR spectroscopy (400 MHz, CDCl₃).

FIG. 17A-J. Effect of engineered translational machinery on the pyridazinone bond formation with 1 and 5. γKPhe (1) and (S)-HzPhe (5) were charged to tRNAfMet(CAU) and tRNAPro1E2(GGU), respectively by Fx and subsequently added to the PURExpress™ system. The 1 and 5 delivered to the ribosome on tRNAfMet(CAU) and tRNAPro1E2(GGU), respectively, were consecutively incorporated into a peptide polymer and permitted to undergo water condensation reactions, yielding a pyridazinone bond. (A-B) The pyridazinone bond was produced either in the presence of the wild-type and an engineered ribosome. (C-D) Peptides containing a pyridazinone at the N-terminus were observed in a low yield when an additional translational machinery, EF-P, was supplemented. Of note, a custom-made Δ (aa, tRNA, ribosome) PURExpress system (NEB, E3315Z) supplying the wild-type ribosome in a separate tube was used for the condensation reactions with engineered ribosomes. The wild-type ribosome supplied in the kit was not used, however, based on previous literature, we expected the engineered 040329 ribosomes to constitute ˜25% of the purified ribosome population. (E-H) The observed mass of each peptide corresponds to the theoretical mass, which is [M+H]⁺=1363; [M+Na]⁺=1385 (see panel J for the structure of theoretical peptide). (I) The percent yield of ribosome-mediated condensation of pyridazninone bond. The yield was obtained based on the relative peak area of the peptide containing a pyridazinone bond shown in the MALDI spectra. (J) Structure of the peptide containing a pyridazinone bond of the N-terminus.

FIG. 18. Map of plasmid pJL1_StrepII. CATATG:
NdeI; GTCGAC: SalI; shaded text:
TGGTCTCATCCGCAGTTCGAAAAA (SEQ ID NO: 8):
strep tag; TAGTAA: stop.
pJL1_MT_StrepII:
[CATATGTCC
TGGTCTCATCCGCAGTTCGAAAAA
TAGTAAGTCGAC (SEQ ID NO: 9)],
fMetThrTrpSerHisProGlnPheGluLys
(SEQ ID NO: 10) (shaded text in FIG. 17 is
represented by bold text).
pJL1_StrepII_TI2:
[CATATG
TGGTCTCATCCGCAGTTCGAAAAA
TCCACCATCACCATCTAGTAAGTCGAC (SEQ ID NO: 11)],
fMetTrpSerHisProGlnPheGluLys
SerThrIleThrIle (SEQ ID NO: 12)
(shaded text in FIG. 17 is represented by
bold text.
pJL1_StrepII_TI3:
[CATATG
TGGTCTCATCCGCAGTTCGAAAAA
TCCACCATCACCATCACCATCTAGTAA
GTCGAC] (SEQ ID NO: 13);
fMetTrpSerHisProGlnPheGluLys
SerThrIleThrIleThrIle (SEQ ID NO: 14)
(shaded text in FIG. 17 is represented by
bold text.)

FIG. 19. Exemplary synthesis scheme of oxaziridine.

FIG. 20. Exemplary synthesis scheme of HzPhe-CME HCl (5).

FIG. 21. Exemplary synthesis scheme of AOP (7).

### DETAILED DESCRIPTION

The presently disclosed subject matter is described herein using several definitions, as set forth below and throughout the application.

### Definitions

Unless defined otherwise, all technical and scientific terms used herein have the same meaning as commonly understood by one of skill in the art to which the invention pertains. Although any methods and materials similar to or equivalent to those described herein can be used in the practice or testing of the present invention, the preferred methods and materials are described herein.

Unless otherwise specified or indicated by context, the terms “a”, “an”, and “the” mean “one or more.” For example, “a component” should be interpreted to mean “one or more components.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

Ranges recited herein include the defined boundary numerical values as well as sub-ranges encompassing any non-recited numerical values within the recited range. For example, a range from about 0.01 mM to about 10.0 mM includes both 0.01 mM and 10.0 mM. Non-recited numerical values within this exemplary recited range also contemplated include, for example, 0.05 mM, 0.10 mM, 0.20 mM, 0.51 mM, 1.0 mM, 1.75 mM, 2.5 mM 5.0 mM, 6.0 mM, 7.5 mM, 8.0 mM, 9.0 mM, and 9.9 mM, among others. Exemplary sub-ranges within this exemplary range include from about 0.01 mM to about 5.0 mM; from about 0.1 mM to about 2.5 mM; and from about 2.0 mM to about 6.0 mM, among others.

### Chemical Entities

New chemical entities and uses for chemical entities are disclosed herein. The chemical entities may be described using terminology known in the art and further discussed below.

As used herein, an asterisk “*” or a plus sign “+” may be used to designate the point of attachment for any radical group or substituent group, for example “R” as discussed herein.

The term “alkyl” as contemplated herein includes a straight-chain or branched alkyl radical in all of its isomeric forms, such as a straight or branched group of 1-12, 1-10, or 1-6 carbon atoms, referred to herein as C1-C12 alkyl, C1-C10-alkyl, and C1-C6-alkyl, respectively.

The term “alkylene” refers to a diradical of straight-chain or branched alkyl group (i.e., a diradical of straight-chain or branched C₁-C₆ alkyl group). Exemplary alkylene groups include, but are not limited to —CH₂—, —CH₂CH₂—, —CH₂CH₂CH₂—, —CH(CH₃)CH₂—, —CH₂CH(CH₃)CH2—, —CH(CH₂CH₃)CH₂—, and the like.

The term “haloalkyl” refers to an alkyl group that is substituted with at least one halogen. For example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). One type of heteroalkyl group is an “alkoxy” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C2-C12-alkenyl, C2-C10-alkenyl, and C2-C6-alkenyl, respectively.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C2-C12-alkynyl, C2-C10-alkynyl, and C2-C6-alkynyl, respectively.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C4-8-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido or carboxyamido, amidino, amino, aryl, arylalkyl, azido, carbamate, carbonate, carboxy, cyano, cycloalkyl, ester, ether, formyl, halo, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloheteroalkyl” refers to a monovalent saturated cyclic, bicyclic, or bridged cyclic hydrocarbon group of 3-12, 3-8, 4-8, or 4-6 carbons in which at least one carbon of the cycloalkane is replaced with a heteroatom such as, for example, N, O, and/or S.

The term “cycloalkylene” refers to a cycloalkyl group that is unsaturated at one or more ring bonds.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number ring carbon atoms. For example, the partially unsaturated carbocyclyl may contain 5-14, 5-12, 5-8, or 5-6 ring carbon atoms, and accordingly be referred to as a 5-14, 5-12, 5-8, or 5-6 membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido or carboxyamido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido or carboxyamido, carboxylic acid, -C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3-to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C3-C7 heterocyclyl group refers to a saturated or partially unsaturated 3- to 7-membered ring structure containing one to four heteroatoms, such as nitrogen, oxygen, and sulfur. The designation “C3-C7” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines (e.g., mono-substituted amines or di-substituted amines), wherein substituents may include, for example, alkyl, cycloalkyl, heterocyclyl, alkenyl, and aryl.

The terms “alkoxy” or “alkoxyl” are art-recognized and refer to an alkyl group, as defined above, having an oxygen radical attached thereto. Representative alkoxy groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

The term “carbonyl” as used herein refers to the radical —C(O)—.

The term “oxo” refers to a divalent oxygen atom —O—.

The term “hydrazineyl” refers to NH₂—NH, NH₂—NH₂ and derivatives including NR2—NR₂, NHR—NR₂, NH₂—NR₂, and NHR—NHR (e.g., wherein R is alkyl).

The term “carboxamido” as used herein refers to the radical —C(O)NRR′, where R and R′ may be the same or different. R and R′, for example, may be independently hydrogen, alkyl, aryl, arylalkyl, cycloalkyl, formyl, haloalkyl, heteroaryl, or heterocyclyl.

The term “carboxy” as used herein refers to the radical —COOH or its corresponding salts, e.g. —COONa, etc.

The term “amide” or “amido” or “amidyl” as used herein refers to a radical of the form —R¹C(O)N(R²)—, —R¹C(O)N(R²)R³—, —C(O)NR₂R³, or —C(O)NH₂, wherein R¹, R² and R³, for example, are each independently hydrogen, alkyl, alkoxy, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” or “+” or “−” depending on the configuration of substituents around the stereogenic carbon atom and or the optical rotation observed. The present invention encompasses various stereo isomers of these compounds and mixtures thereof. Stereoisomers include enantiomers and diastereomers. Mixtures of enantiomers or diastereomers may be designated (±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise. Also contemplated herein are compositions comprising, consisting essentially of, or consisting of an enantiopure compound, which composition may comprise, consist essential of, or consist of at least about 50%, 60%, 70%, 80%, 90%, 95%, 96%, 97%, 98%, 99%, or 100% of a single enantiomer of a given compound (e.g., at least about 99% of an R enantiomer of a given compound).

### Nucleic Acids and Reactions

The terms “nucleic acid” and “oligonucleotide,” as used herein, refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present invention, an oligonucleotide also can comprise nucleotide analogs in which the base, sugar, or phosphate backbone is modified as well as non-purine or non-pyrimidine nucleotide analogs.

Oligonucleotides can be prepared by any suitable method, including direct chemical synthesis by a method such as the phosphotriester method of Narang et al., 1979, Meth. Enzymol. 68:90-99; the phosphodiester method of Brown et al., 1979, Meth. Enzymol. 68:109-151; the diethylphosphoramidite method of Beaucage et al., 1981, Tetrahedron Letters 22:1859-1862; and the solid support method of U.S. Pat. No. 4,458,066, each incorporated herein by reference. A review of synthesis methods of conjugates of oligonucleotides and modified nucleotides is provided in Goodchild, 1990, Bioconjugate Chemistry 1(3): 165-187, incorporated herein by reference.

The term “amplification reaction” refers to any chemical reaction, including an enzymatic reaction, which results in increased copies of a template nucleic acid sequence or results in transcription of a template nucleic acid. Amplification reactions include reverse transcription, the polymerase chain reaction (PCR), including Real Time PCR (see U.S. Pat. Nos. 4,683,195 and 4,683,202; PCR Protocols: A Guide to Methods and Applications (Innis et al., eds, 1990)), and the ligase chain reaction (LCR) (see Barany et al., U.S. Pat. No. 5,494,810). Exemplary “amplification reactions conditions” or “amplification conditions” typically comprise either two or three step cycles. Two-step cycles have a high temperature denaturation step followed by a hybridization/elongation (or ligation) step. Three step cycles comprise a denaturation step followed by a hybridization step followed by a separate elongation step.

The terms “target, “target sequence”, “target region”, and “target nucleic acid,” as used herein, are synonymous and refer to a region or sequence of a nucleic acid which is to be amplified, sequenced, or detected. The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, New York; Wetmur, 1991, Critical Review in Biochem. and Mol. Biol. 26(3/4):227-259; and Owczarzy et al., 2008, Biochemistry, 47: 5336-5353, which are incorporated herein by reference).

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

A primer is preferably a single-stranded DNA. The appropriate length of a primer depends on the intended use of the primer but typically ranges from about 6 to about 225 nucleotides, including intermediate ranges, such as from 15 to 35 nucleotides, from 18 to 75 nucleotides and from 25 to 150 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein. Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning or detection of the amplified product, or which enables transcription of RNA (for example, by inclusion of a promoter) or translation of protein (for example, by inclusion of a 5′-UTR, such as an Internal Ribosome Entry Site (IRES) or a 3′-UTR element, such as a poly(A)n sequence, where n is in the range from about 20 to about 200). The region of the primer that is sufficiently complementary to the template to hybridize is referred to herein as the hybridizing region.

As used herein, a primer is “specific,” for a target sequence if, when used in an amplification reaction under sufficiently stringent conditions, the primer hybridizes primarily to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in many cases. Hybridization conditions can be chosen under which the primer can form stable duplexes only with a target sequence. Thus, the use of target-specific primers under suitably stringent amplification conditions enables the selective amplification of those target sequences that contain the target primer binding sites.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, bacteriophage polymerases such as, but not limited to, T3 RNA polymerase, T7 RNA polymerase, SP6 RNA polymerase and E. coli RNA polymerase, among others. The foregoing examples of RNA polymerases are also known as DNA-dependent RNA polymerase. The polymerase activity of any of the above enzymes can be determined by means well known in the art.

The term “promoter” refers to a cis-acting DNA sequence that directs RNA polymerase and other trans-acting transcription factors to initiate RNA transcription from the DNA template that includes the cis-acting DNA sequence.

As used herein, the term “sequence defined biopolymer” refers to a biopolymer having a specific primary sequence. A sequence defined biopolymer can be equivalent to a genetically-encoded defined biopolymer in cases where a gene encodes the biopolymer having a specific primary sequence.

As used herein, “expression template” refers to a nucleic acid that serves as substrate for transcribing at least one RNA that can be translated into a sequence defined biopolymer (e.g., a polypeptide or protein). Expression templates include nucleic acids composed of DNA or RNA. Suitable sources of DNA for use a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms. As used herein, “expression template” and “transcription template” have the same meaning and are used interchangeably.

As used herein, “translation template” refers to an RNA product of transcription from an expression template that can be used by ribosomes to synthesize polypeptide or protein.

As used herein, coupled transcription/translation (“Tx/Tl”), refers to the de novo synthesis of both RNA and a sequence defined biopolymer from the same extract. For example, coupled transcription/translation of a given sequence defined biopolymer can arise in an extract containing an expression template and a polymerase capable of generating a translation template from the expression template. Coupled transcription/translation can occur using a cognate expression template and polymerase from the organism used to prepare the extract. Coupled transcription/translation can also occur using exogenously-supplied expression template and polymerase from an orthogonal host organism different from the organism used to prepare the extract. In the case of an extract prepared from a yeast organism, an example of an exogenously-supplied expression template includes a translational open reading frame operably coupled a bacteriophage polymerase-specific promoter and an example of the polymerase from an orthogonal host organism includes the corresponding bacteriophage polymerase.

The term “reaction mixture,” as used herein, refers to a solution containing reagents necessary to carry out a given reaction. An “amplification reaction mixture”, which refers to a solution containing reagents necessary to carry out an amplification reaction, typically contains oligonucleotide primers and a DNA polymerase in a suitable buffer. A “PCR reaction mixture” typically contains oligonucleotide primers, a DNA polymerase (most typically a thermostable DNA polymerase), dNTPs, and a divalent metal cation in a suitable buffer.

### Cell-Free Protein Synthesis (CFPS)

The disclosed subject matter relates in part to methods, systems, components, and compositions for cell-free protein synthesis. Cell-free protein synthesis (CFPS) is known and has been described in the art. (See, e.g., U.S. Pat. Nos. 6,548,276; 7,186,525; 8,734,856; 7,235,382; 7,273,615; 7,008,651; 6,994,986 7,312,049; 7,776,535; 7,817,794; 8,298,759; 8,715,958; 9,005,920; U.S. Publication No. 2014/0349353, and U.S. Publication No. 2016/0060301, the contents of which are incorporated herein by reference in their entireties). A “CFPS reaction mixture” typically contains a crude or partially-purified bacterial or yeast extract, an RNA translation template, and a suitable reaction buffer for promoting cell-free protein synthesis from the RNA translation template. In some aspects, the CFPS reaction mixture can include exogenous RNA translation template. In other aspects, the CFPS reaction mixture can include a DNA expression template encoding an open reading frame operably linked to a promoter element for a DNA-dependent RNA polymerase. In these other aspects, the CFPS reaction mixture can also include a DNA-dependent RNA polymerase to direct transcription of an RNA translation template encoding the open reading frame. In these other aspects, additional NTP's and divalent cation cofactor can be included in the CFPS reaction mixture. A reaction mixture is referred to as complete if it contains all reagents necessary to enable the reaction, and incomplete if it contains only a subset of the necessary reagents. It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components may be packaged separately for commercialization and that useful commercial kits may contain any subset of the reaction components of the invention.

### Platforms for Preparing Sequence Defined Biopolymers

An aspect of the invention is a platform for preparing a sequence defined biopolymer or protein in vitro. The platform for preparing a sequence defined polymer or protein in vitro comprises a cellular extract from bacterial or eukaryotic (e.g., yeast, or mammalian cells) organism as described above. Because CFPS exploits an ensemble of catalytic proteins prepared from the crude lysate of cells, the cell extract (whose composition is sensitive to growth media, lysis method, and processing conditions) is an important component of extract-based CFPS reactions. A variety of methods exist for preparing an extract competent for cell-free protein synthesis, including U.S. patent application Ser. No. 14/213,390 to Michael C. Jewett et al., entitled METHODS FOR CELL-FREE PROTEIN SYNTHESIS, filed Mar. 14, 2014, and now published as U.S. Patent Application Publication No. 2014/0295492 on Oct. 2, 2014, and U.S. patent application Ser. No. 14/840,249 to Michael C. Jewett et al., entitled METHODS FOR IMPROVED IN VITRO PROTEIN SYNTHESIS WITH PROTEINS CONTAINING NON STANDARD AMINO ACIDS, filed Aug. 31, 2015, and now published as U.S. Patent Application Publication No. 2016/0060301, on Mar. 3, 2016, the contents of which are incorporated by reference.

The platform may comprise an expression template, a translation template, or both an expression template and a translation template. The expression template serves as a substrate for transcribing at least one RNA that can be translated into a sequence defined biopolymer (e.g., a polypeptide or protein). The translation template is an RNA product that can be used by ribosomes to synthesize the sequence defined biopolymer. In certain embodiments the platform comprises both the expression template and the translation template. In certain specific embodiments, the platform may be a coupled transcription/translation (“Tx/Tl”) system where synthesis of translation template and a sequence defined biopolymer from the same cellular extract.

The platform may comprise one or more polymerases capable of generating a translation template from an expression template. The polymerase may be supplied exogenously or may be supplied from the organism used to prepare the extract. In certain specific embodiments, the polymerase is expressed from a plasmid present in the organism used to prepare the extract and/or an integration site in the genome of the organism used to prepare the extract.

The platform may comprise an orthogonal translation system. An orthogonal translation system may comprise one or more orthogonal components that are designed to operate parallel to and/or independent of the organism's orthogonal translation machinery. In certain embodiments, the orthogonal translation system and/or orthogonal components are configured to incorporation of unnatural amino acids. An orthogonal component may be an orthogonal protein or an orthogonal RNA. In certain embodiments, an orthogonal protein may be an orthogonal synthetase. In certain embodiments, the orthogonal RNA may be an orthogonal tRNA or an orthogonal rRNA. An example of an orthogonal rRNA component has been described in Application No. PCT/US2015/033221 to Michael C. Jewett et al., entitled TETHERED RIBOSOMES AND METHODS OF MAKING AND USING THEREOF, filed 29 May 2015, and now published as WO2015184283, and U.S. patent application Ser. No. 15/363,828, to Michael C. Jewett et al., entitled RIBOSOMES WITH TETHERED SUBUNITS, filed on November 29, 2016, and now published as U.S. Patent Application Publication No. 2017/0073381, on March 16, 2017, the contents of which are incorporated by reference. In certain embodiments, one or more orthogonal components may be prepared in vivo or in vitro by the expression of an oligonucleotide template. The one or more orthogonal components may be expressed from a plasmid present in the genomically recoded organism, expressed from an integration site in the genome of the genetically recoded organism, co-expressed from both a plasmid present in the genomically recoded organism and an integration site in the genome of the genetically recoded organism, express in the in vitro transcription and translation reaction, or added exogenously as a factor (e.g., a orthogonal tRNA or an orthogonal synthetase added to the platform or a reaction mixture).

Altering the physicochemical environment of the CFPS reaction to better mimic the cytoplasm can improve protein synthesis activity. The following parameters can be considered alone or in combination with one or more other components to improve robust CFPS reaction platforms based upon crude cellular extracts.

The temperature may be any temperature suitable for CFPS. Temperature may be in the general range from about 10° C. to about 40° C., including intermediate specific ranges within this general range, include from about 15° C. to about 35° C., form about 15° C. to about 30° C., form about 15° C. to about 25° C. In certain aspects, the reaction temperature can be about 15° C., about 16° C., about 17° C., about 18° C., about 19° C., about 20° C., about 21° C., about 22° C., about 23° C., about 24° C., about 25° C.

The CFPS reaction can include any organic anion suitable for CFPS. In certain aspects, the organic anions can be glutamate, acetate, among others. In certain aspects, the concentration for the organic anions is independently in the general range from about 0 mM to about 200 mM, including intermediate specific values within this general range, such as about 0 mM, about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM and about 200 mM, among others.

The CFPS reaction can also include any halide anion suitable for CFPS. In certain aspects the halide anion can be chloride, bromide, iodide, among others. A preferred halide anion is chloride. Generally, the concentration of halide anions, if present in the reaction, is within the general range from about 0 mM to about 200 mM, including intermediate specific values within this general range, such as those disclosed for organic anions generally herein.

The CFPS reaction may also include any organic cation suitable for CFPS. In certain aspects, the organic cation can be a polyamine, such as spermidine or putrescine, among others. Preferably polyamines are present in the CFPS reaction. In certain aspects, the concentration of organic cations in the reaction can be in the general about 0 mM to about 3 mM, about 0.5 mM to about 2.5 mM, about 1 mM to about 2 mM. In certain aspects, more than one organic cation can be present.

The CFPS reaction can include any inorganic cation suitable for CFPS. For example, suitable inorganic cations can include monovalent cations, such as sodium, potassium, lithium, among others; and divalent cations, such as magnesium, calcium, manganese, among others. In certain aspects, the inorganic cation is magnesium. In such aspects, the magnesium concentration can be within the general range from about 1 mM to about 50 mM, including intermediate specific values within this general range, such as about 1 mM, about 2 mM, about 3 mM, about 5 mM, about 6 mM, about 7 mM, about 8 mM, about 9 mM, about 10 mM, among others. In preferred aspects, the concentration of inorganic cations can be within the specific range from about 4 mM to about 9 mM and more preferably, within the range from about 5 mM to about 7 mM.

The CFPS reaction includes NTPs. In certain aspects, the reaction use ATP, GTP, CTP, and UTP. In certain aspects, the concentration of individual NTPs is within the range from about 0.1 mM to about 2 mM.

The CFPS reaction can also include any alcohol suitable for CFPS. In certain aspects, the alcohol may be a polyol, and more specifically glycerol. In certain aspects the alcohol is between the general range from about 0% (v/v) to about 25% (v/v), including specific intermediate values of about 5% (v/v), about 10% (v/v) and about 15% (v/v), and about 20% (v/v), among others.

### Methods for Preparing Proteins and Sequence Defined Biopolymers

An aspect of the invention is a method for cell-free protein synthesis of a sequence defined biopolymer or protein in vitro. The method comprises contacting an RNA template encoding a sequence defined biopolymer with a reaction mixture comprising a cellular extract as described above. Methods for cell-free protein synthesis of a sequence defined biopolymers have been described [1, 18, 26].

In certain embodiments, a sequence-defined biopolymer or protein comprises a product prepared by the method or the platform that includes an amino acid. In certain embodiments the amino acid may be a natural amino acid. As used herein a natural amino acid is a proteinogenic amino acid encoded directly by a codon of the universal genetic code. In certain embodiments the amino acid may be an unnatural amino acid. As used here an unnatural amino acid is a nonproteinogenic amino acid. An unnatural amino acid may also be referred to as a non-standard amino acid (NSAA) or non-canonical amino acid. In certain embodiments, a sequence defined biopolymer or protein may comprise a plurality of unnatural amino acids. In certain specific embodiments, a sequence defined biopolymer or protein may comprise a plurality of the same unnatural amino acid. The sequence defined biopolymer or protein may comprise at least 5, at least 10, at least 15, at least 20, at least 25, at least 30, at least 35, or at least 40 or the same or different unnatural amino acids.

Examples of unnatural, non-canonical, and/or non-standard amino acids include, but are not limited, to a p-acetyl-L-phenylalanine, a p-iodo-L-phenylalanine, an O-methyl-L-tyrosine, a p-propargyloxyphenylalanine, a p-propargyl-phenylalanine, an L-3-(2-naphthyl)alanine, a 3-methyl-phenylalanine, an O-4-allyl-L-tyrosine, a 4-propyl-L-tyrosine, a tri-O-acetyl-GlcNAcpβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an unnatural analogue of a methionine amino acid; an unnatural analogue of a leucine amino acid; an unnatural analogue of a isoleucine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, 24ufa24hor, phosphono, phosphine, heterocyclic, enone, imine, aldehyde, hydroxylamine, keto, or amino substituted amino acid, or a combination thereof; an amino acid with a photoactivatable cross-linker; a spin-labeled amino acid; a fluorescent amino acid; a metal binding amino acid; a metal-containing amino acid; a radioactive amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an a-hydroxy containing acid; an amino thio acid; an α,α disubstituted amino acid; a β-amino acid; a γ-amino acid, a cyclic amino acid other than proline or histidine, and an aromatic amino acid other than phenylalanine, tyrosine or tryptophan.

The methods described herein allow for preparation of sequence defined biopolymers or proteins with high fidelity to a RNA template. In other words, the methods described herein allow for the correct incorporation of unnatural, non-canonical, and/or non-standard amino acids as encoded by an RNA template. In certain embodiments, the sequence defined biopolymer encoded by a RNA template comprises at least 5, at least 10, at least 15, at least 20, at least 25, at least 30, at least 35, or at least 40 unnatural, non-canonical, and/or non-standard amino acids and a product prepared from the method includes at least 80%, at least 85%, at least 90%, at least 95%, or 100% of the encoded unnatural, non-canonical, and/or non-standard amino acids.

The methods described herein also allow for the preparation of a plurality of products prepared by the method. In certain embodiments, at least 80%, at least 85%, at least 90%, at least 95%, or at least 98% of a plurality of products prepared by the method are full length. In certain embodiments, the sequence defined biopolymer encoded by a RNA template comprises at least 5, at least 10, at least 15, at least 20, at least 25, at least 30, at least 35, or at least 40 unnatural, non-canonical, and/or non-standard amino acids and at least 80%, at least 85%, at least 90%, at least 95%, or at least 98% of a plurality of products prepared by the method include 100% of the encoded unnatural, non-canonical, and/or non-standard amino acids.

In certain embodiments, the sequence defined biopolymer or the protein encodes a therapeutic product, a diagnostic product, a biomaterial product, an adhesive product, a biocomposite product, or an agricultural product.

### Ribosome-Mediated Synthesis of Polymers Comprising Non-Amide, Non-Ester Bonds to Join Substrate Molecules

Ribosome-mediated polymerization is a powerful technology due to the ribosome's ability to polymerize monomers at a rapid rate (20 aa/sec) and high fidelity (99.999%). However, the type of polymers that can be produced by the ribosome has been mostly confined to polyamide analogues because natural ribosomes have been evolutionarily optimized to form a peptide (amide) bond between monomers. Here, we rationally design a variety of non-canonical chemical substrates that could form a non-amide polymer backbone when site specifically incorporated into a peptide and demonstrate that the ribosome enables the formation of 5-, 6-, and 7-membered heterocyclic structures such as pyrazolone, pyridazinone, and diazepinone. We optimize the bond formation reaction using an engineered ribosome and translation factors and show the engineered ribosome produced the bond more efficiently than the wild type ribosome.

Moreover, we expanded the range of non-canonical substrates into oxo- and hydrazineyl acid substrates and present a wide variety of heterocyclic ring derivatives that are produced under the optimized reaction condition. We finally show consecutive incorporation of these monomers into a peptide, and produce various polymers containing multiple cyclic bonds. This suggests that our ribosome-mediated polymerization approach can be a transformative technology to produce alternating block copolymers such as AB, ABA, or ABAB.

The system and compounds disclosed herein allow for cell-free ribosome-mediated synthesis of polymers containing non-amide, non-ester bonds joining the substrates. In lieu of amide or ester bonds, the systems and compounds disclosed herein provide for the formation of heterocyclic covalent bonds.

We reprogrammed tRNAs with non-canonical chemical substrates using a ribozyme that charges the substrates into the 3′-hydroxyl group of synthetic tRNA. We added the reassigned tRNAs into a cell-free system, where the tRNAs are delivered to the ribosome by translation factors. The non-canonical substrates react with each other and form heterocyclic products in the ribosome. We used the ribosome that has been evolutionarily optimized to synthesize peptides and proteins as a chemical machine to produce these chemical bonds.

### Substrates

In addition to the canonical and non-canonical amino acids described above, disclosed herein are substrates for ribosome-medicated polymer production. The substrates are charged to tRNAs by Fx, and utilized as monomers that are linked together by ribosomes in cell-free extracts to form polymers. The monomers are linked by non-amide, non-ester bonds. In some embodiments, the bonds are heterocyclic covalent bonds, formed by the ribosomes.

The range of monomer building blocks has been confined to substrates with one nucleophile and one electrophile (e.g. amino acid, hydroxy acid, thioacid). As disclosed herein, the inventors have expanded the building blocks into the substrates with more than two nucleophiles and electrophiles. For example, the inventors expanded the range of non-canonical substrates into oxo- and hydrazineyl acid substrates, e.g., as shown in FIGS. 2 and 4.

In some embodiments, the novel substrates for use in the methods disclosed herein may have a Formula I or Formula II:

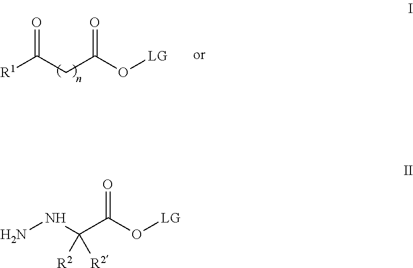

wherein:
n is 1-10, 1-9, 1-8, 1-7, 1-6, 1-5, 1-4, or 1-3;
R¹ is hydrogen, alkyl (e.g., methyl, ethyl), aryl (e.g., phenyl) which optionally is substituted at one or more positions with alkyl or alkylthio (e.g., 4-methylthio-phenyl),
R² is hydrogen, alkyl (e.g., methyl, isopropyl), alkylaryl (e.g., benzyl) which optionally is substituted at one or more positions with hydroxyl (e.g., 3,4-dihydroxy-benzyl), or R2 is the side chain of an amino acid (e.g., a side chain of an amino acid selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine;
R^{2′} is hydrogen or alkyl (e.g., methyl); and
LG is a leaving group that is removed when the chemical moiety is utilized to acylate a tRNA molecule (e.g., when the chemical moiety is utilized to acylate a tRNA molecule at the C3 hydroxyl group) and form an acylated tRNA having a Formula I(a) or II(a):

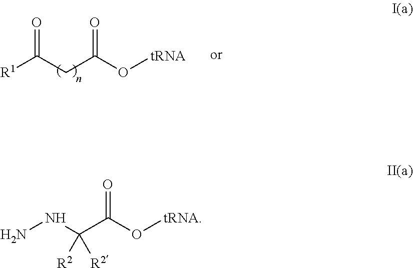

In some embodiments, the disclosed substrates are utilized to form acylated tRNAs

having a formula selected from:

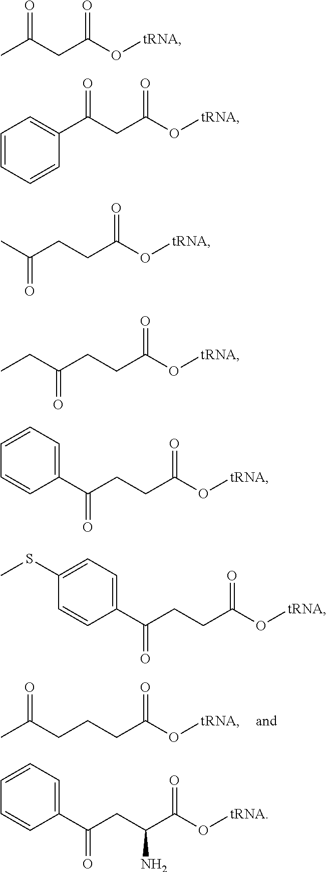

In other embodiments, the disclosed substrates are utilized to prepare acylated tRNAs having a formula selected from:

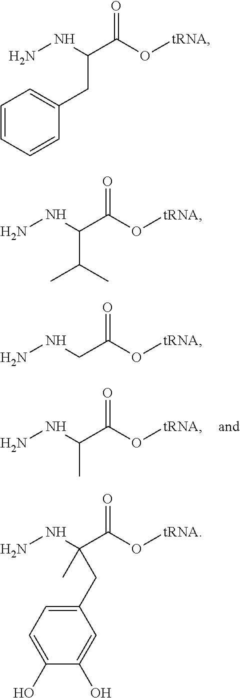

In some embodiments, the disclosed substrates and/or tRNAs comprising the disclosed

substrates may be utilized to prepare compounds or molecules having a Formula III:

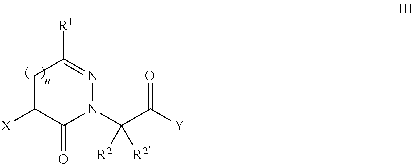

wherein:
X is hydrogen or the C-terminus of a polymer chain (e.g., the C-terminus of a polypeptide chain);
n is 0-8, 0-7, 0-6, 0-5, 0-4, 0-3, or 0-2;
R¹ is hydrogen, alkyl (e.g., methyl, ethyl), aryl (e.g., phenyl) which optionally is substituted at one or more positions with alkyl or alkylthio (e.g., 4-methylthio-phenyl), or R¹ is the C-terminus of a peptide chain;
R² is hydrogen, alkyl (e.g., methyl, isopropyl), alkylaryl (e.g., benzyl) which optionally is substituted at one or more positions with hydroxyl (e.g., 3,4-dihydroxy-benzyl), or R² is the side chain of an amino acid (e.g., a side chain of an amino acid selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine;
R² is hydrogen or alkyl (e.g., methyl);
Y is hydrogen or the N-terminus of a polymer chain (e.g., the N-terminus of a polypeptide chain) or Y has a formula selected from —O(tRNA), —O(R³), or —NH(R³), wherein R³ is selected from hydrogen and alkyl.

### Fx Enzymes

In the preparation method, Fx catalyzes an acylation reaction between the 3′ terminal ribonucleotide of the tRNA and the donor molecule to prepare the acylated tRNA molecule (e.g. via an ester bond formed with the ribose of a 3′ terminal adenosine of the tRNA molecule and the R moiety).

Any suitable Fx may be utilized in the disclosed preparation methods. Suitable Fx's may include, but are not limited to aFx, dFx, and eFx.

tRNA

Any suitable tRNA may be utilized in the preparation methods to generate a compound comprising an acylated tRNA, e.g., an acylated tRNA comprising the compound of formula I or II. Suitable tRNA molecules for the preparation methods may include, but are not limited to, tRNA molecules comprising anticodons corresponding to any of the natural amino acids. By way of example, only, in some embodiments, the tRNA comprises the anticodon CAU (i.e., the anticodon for methionine). In other embodiments, the tRNA comprises the anticodon GGU (i.e., an anticodon for threonine), the anticodon GAU (i.e., an anticodon for isoleucine), the anticodon GGC (i.e., an anticodon for alanine), or the anticodon CAU (i.e., an anticodon for methionine).

The disclosed preparation methods are performed under conditions that maximize the yield of acylated tRNA. In some embodiments, the preparation methods are performed under reaction conditions such that at least about 50% of the tRNA in the reaction mixture is acylated after reacting the reaction mixture for 120 hours, and preferably under reaction conditions such that at least about 50% of the tRNA in the reaction mixture is acylated after reacting the reaction mixture for 16 hours.

### Engineered Ribosomes

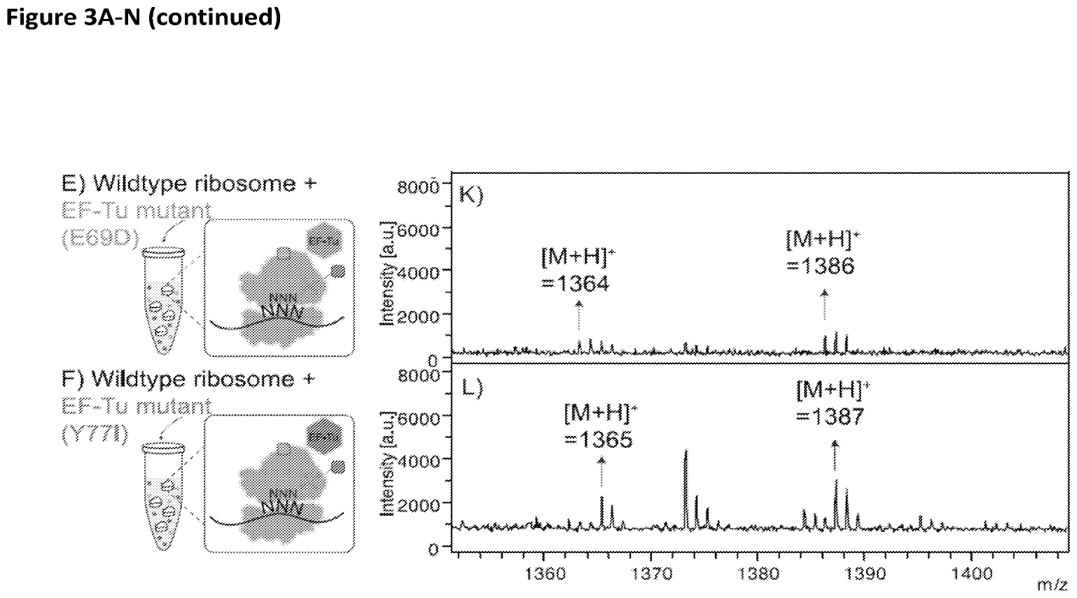

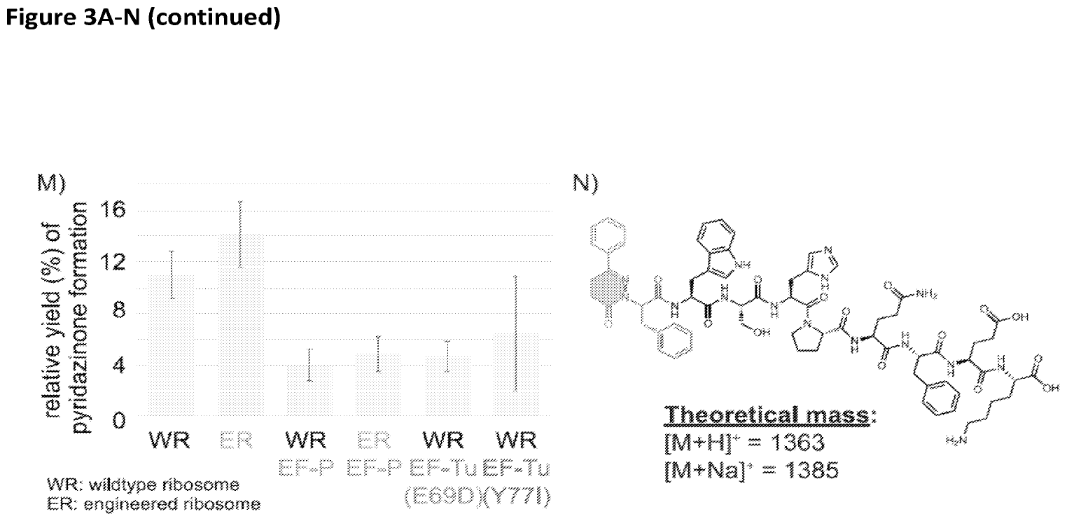

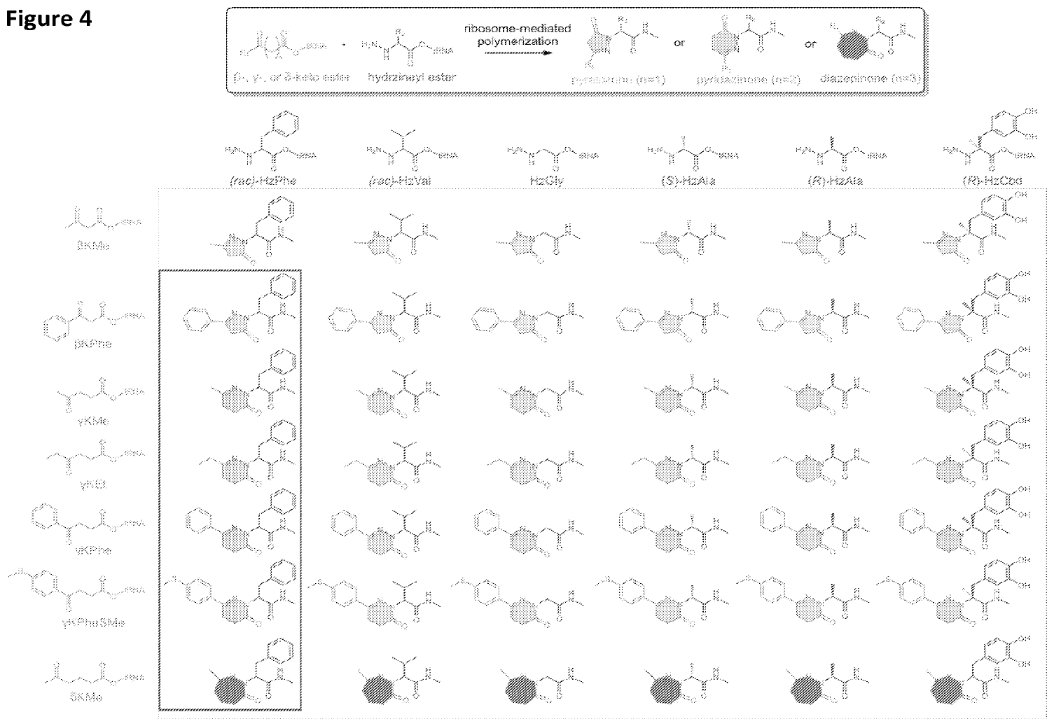

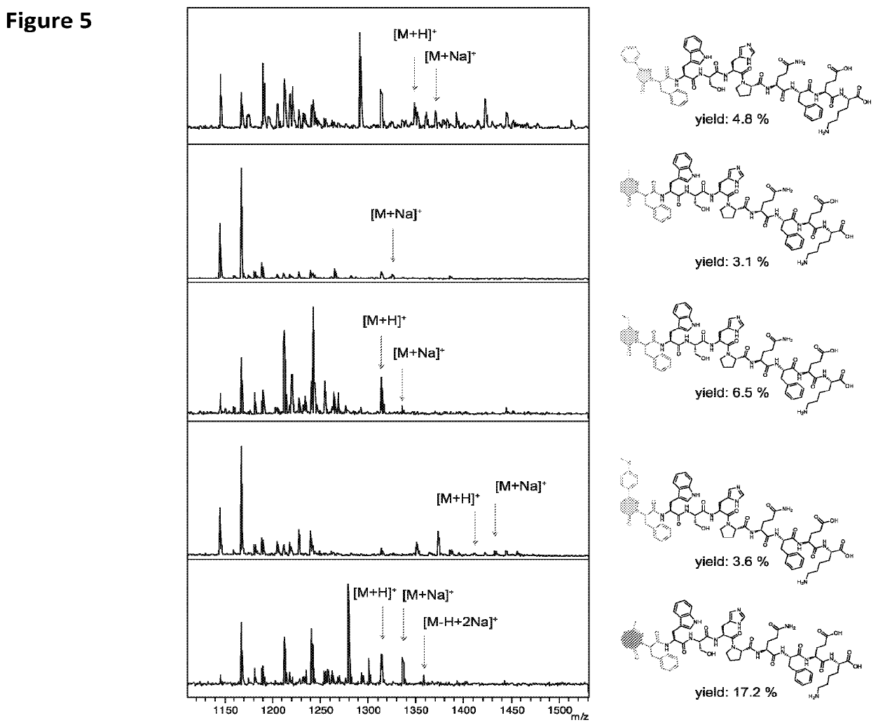

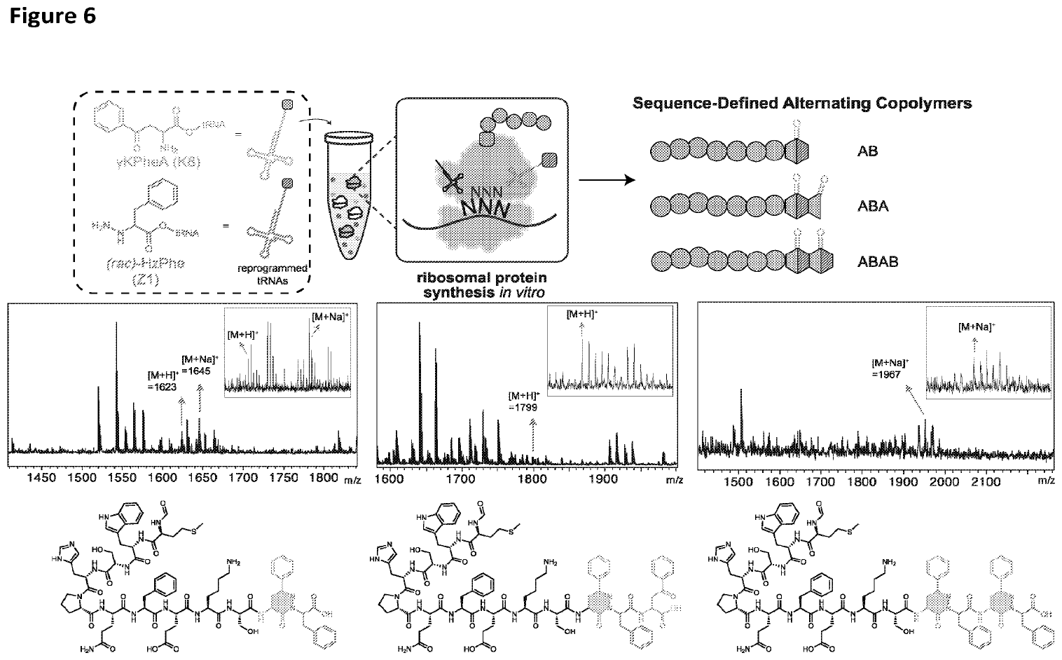

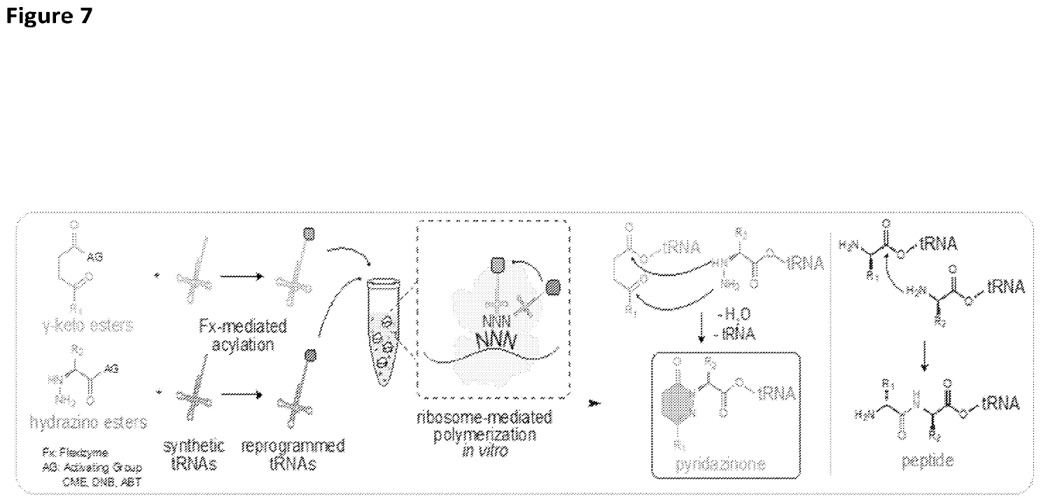

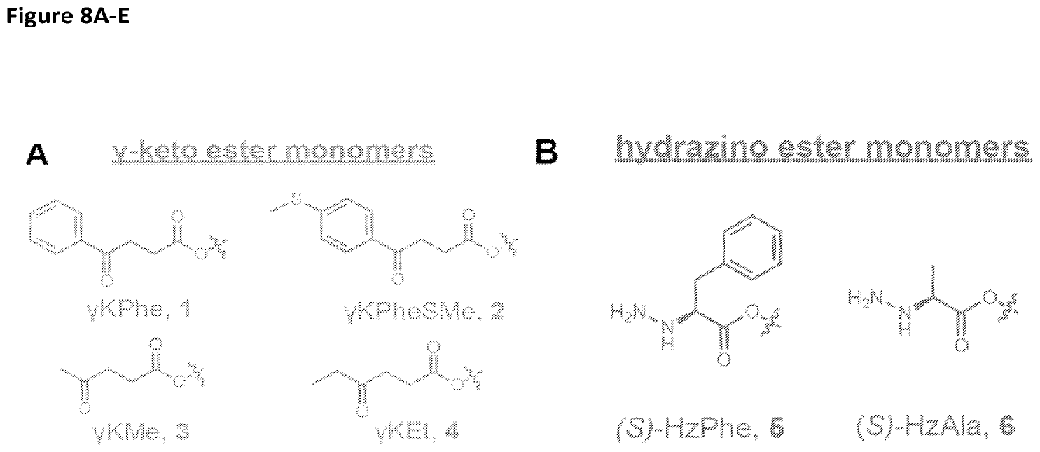

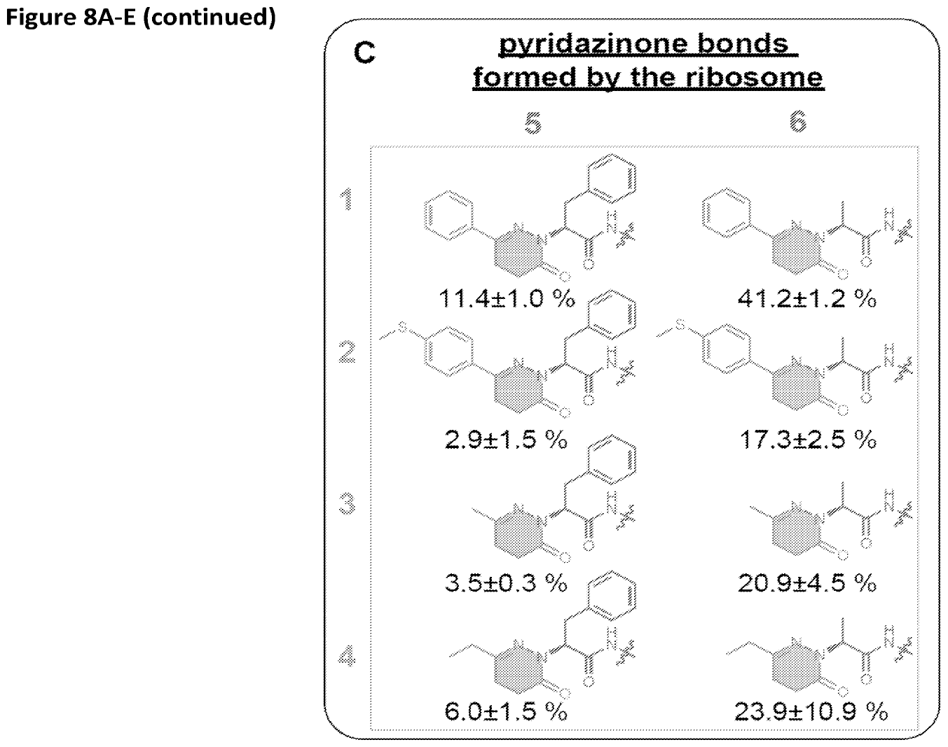

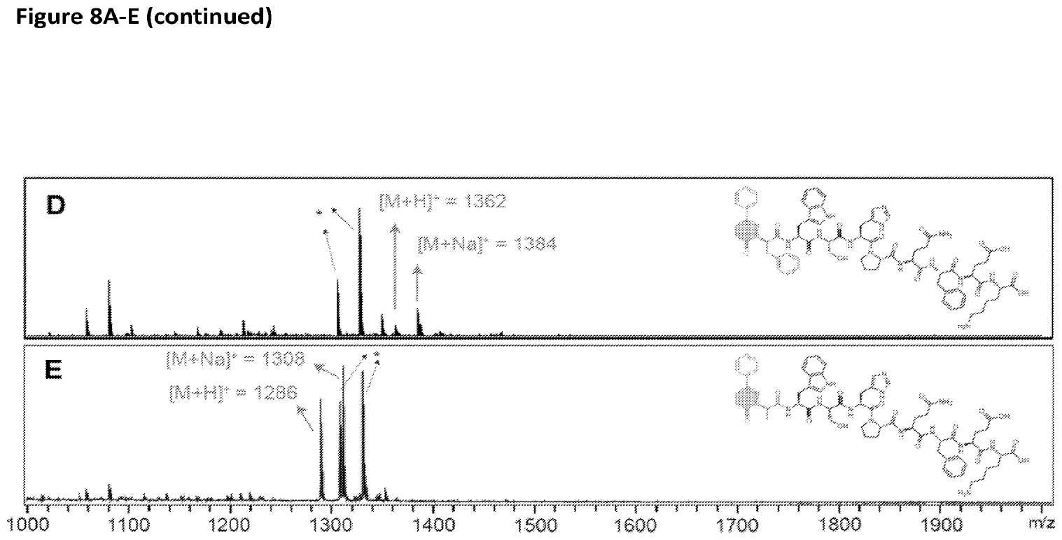

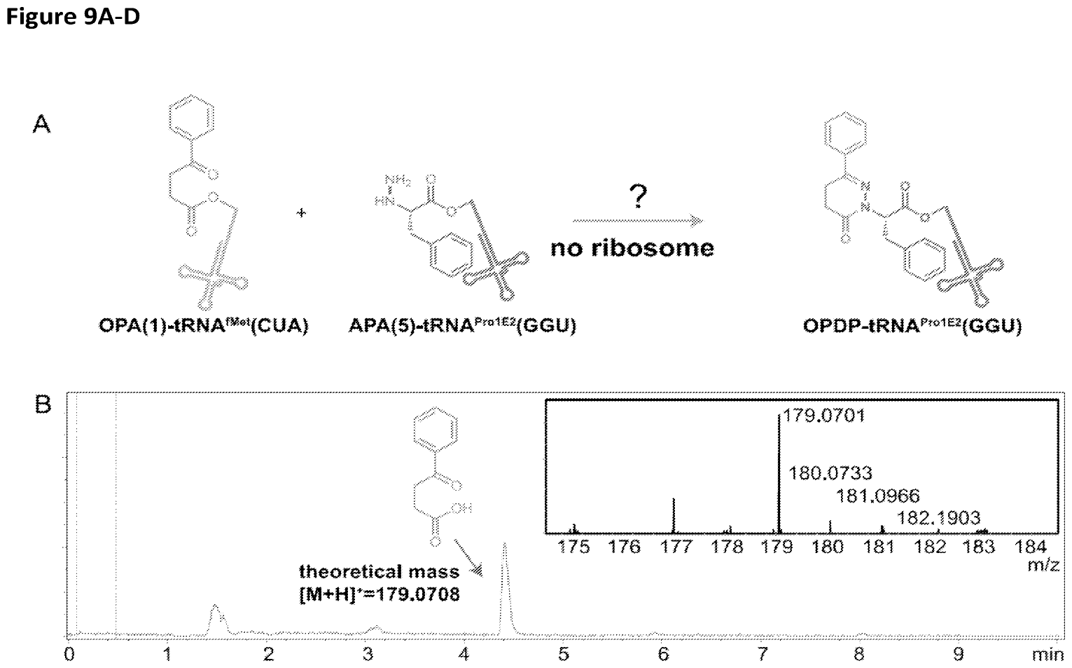

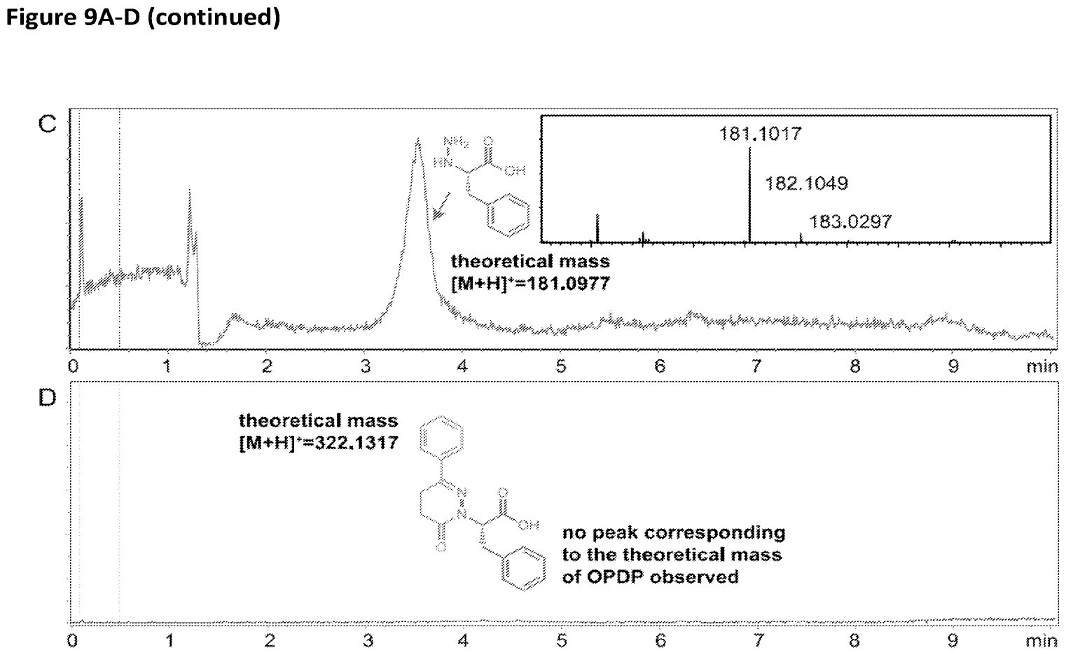

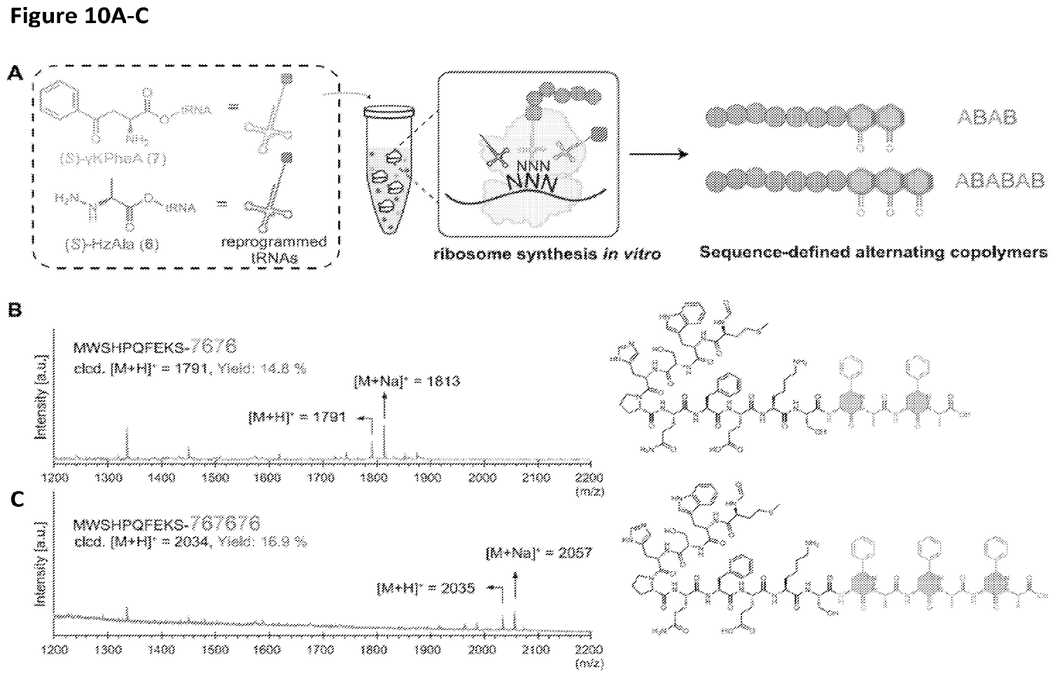

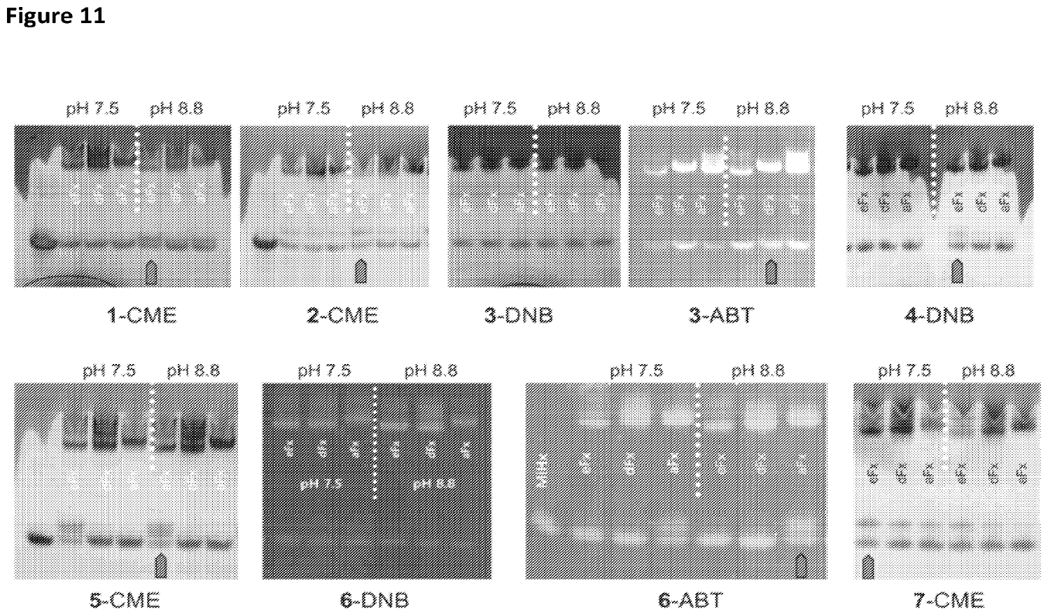

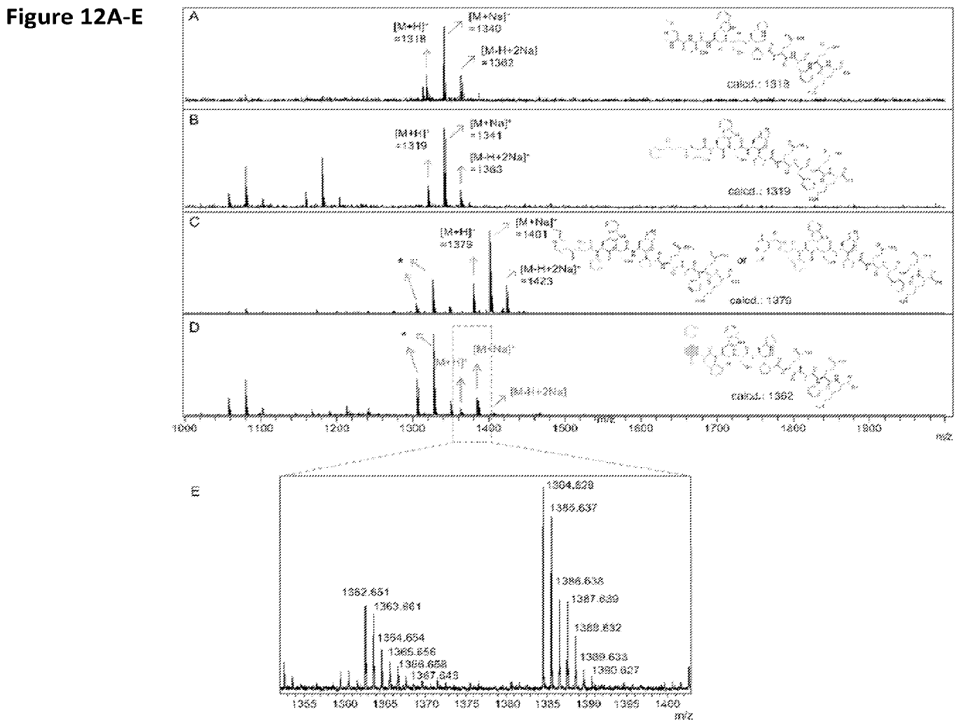

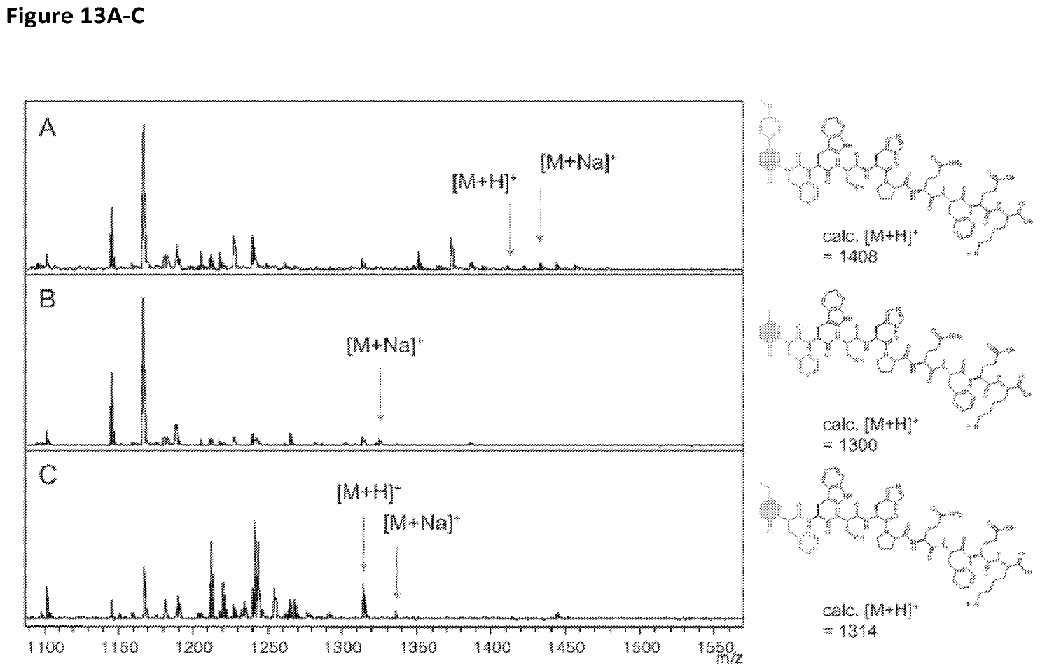

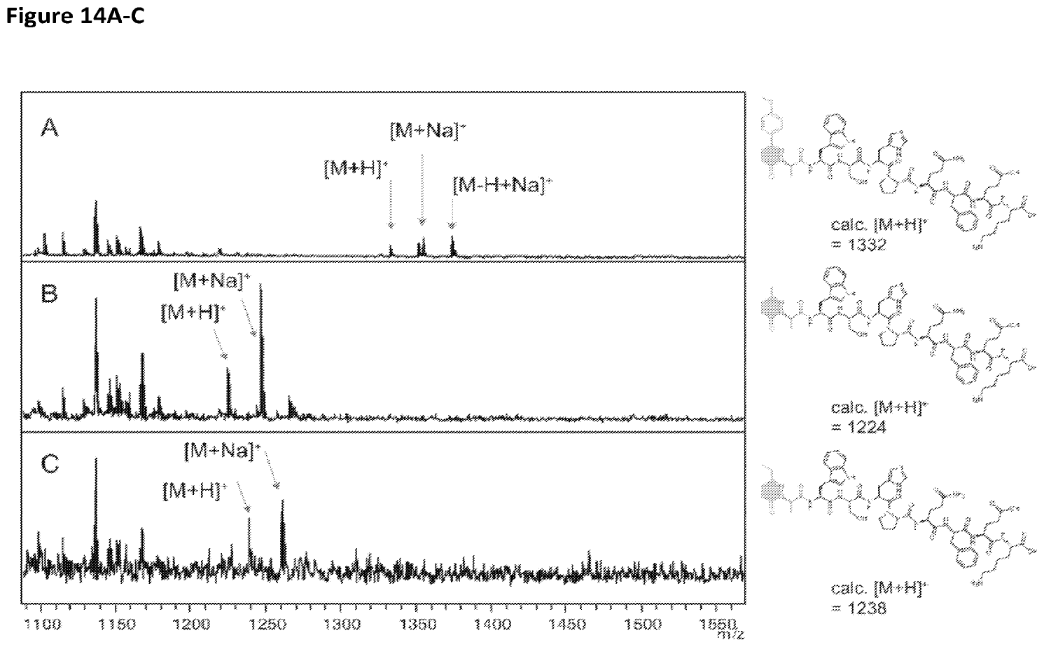

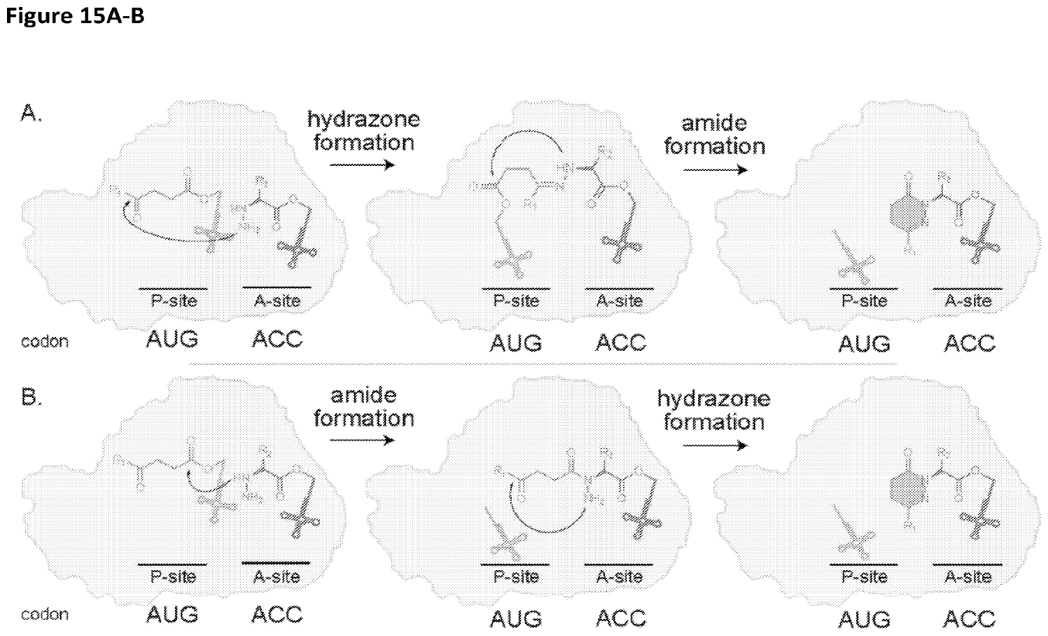

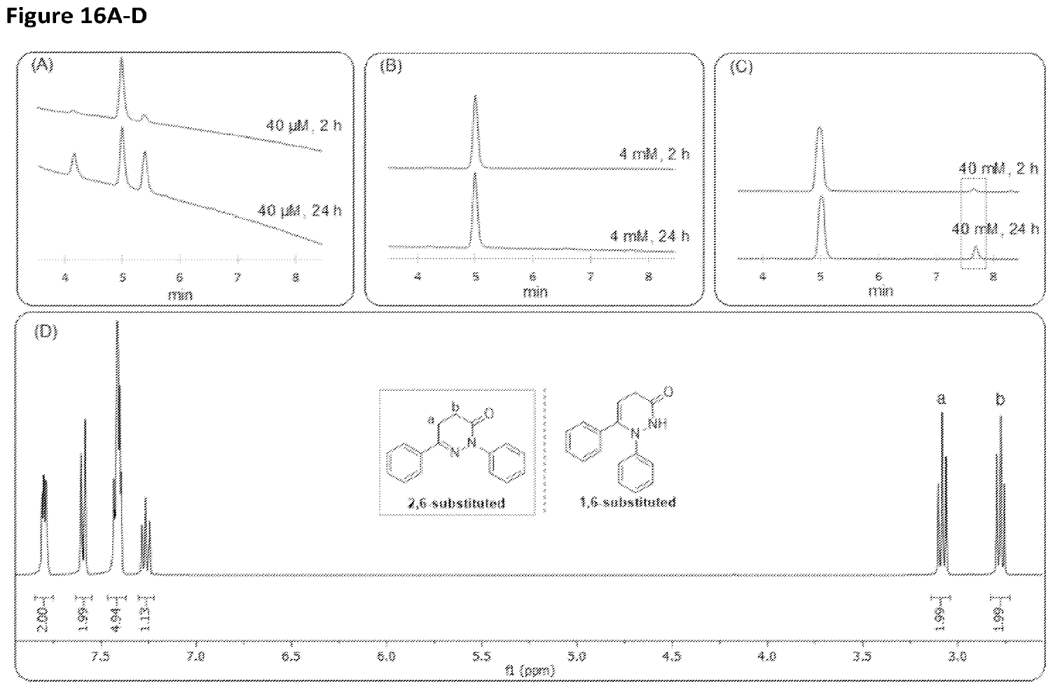

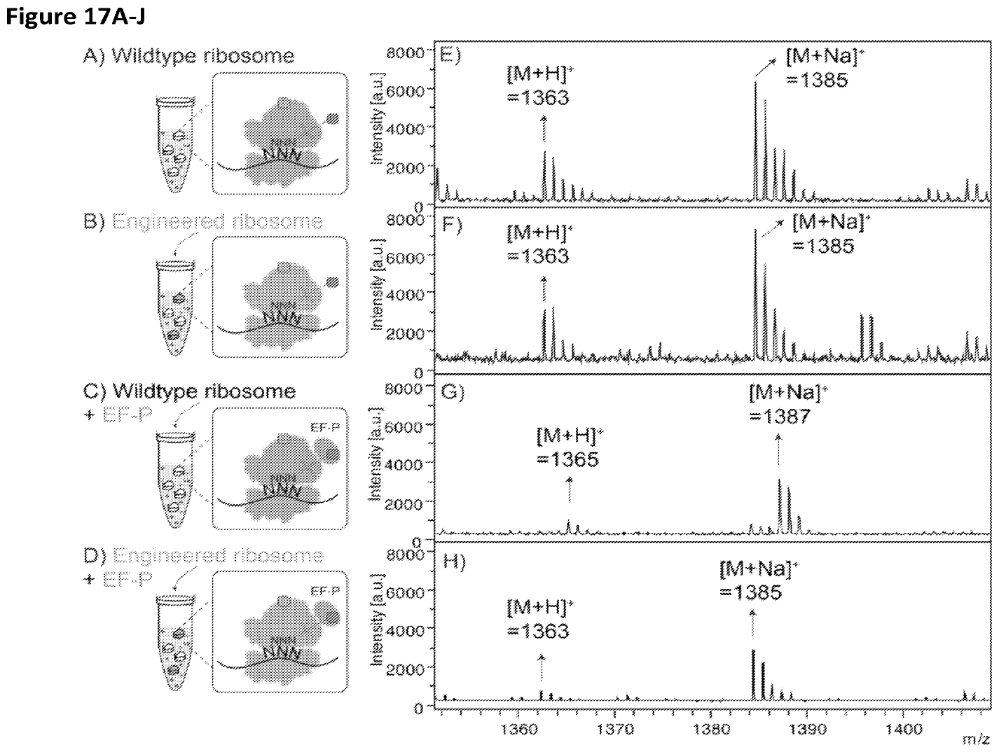

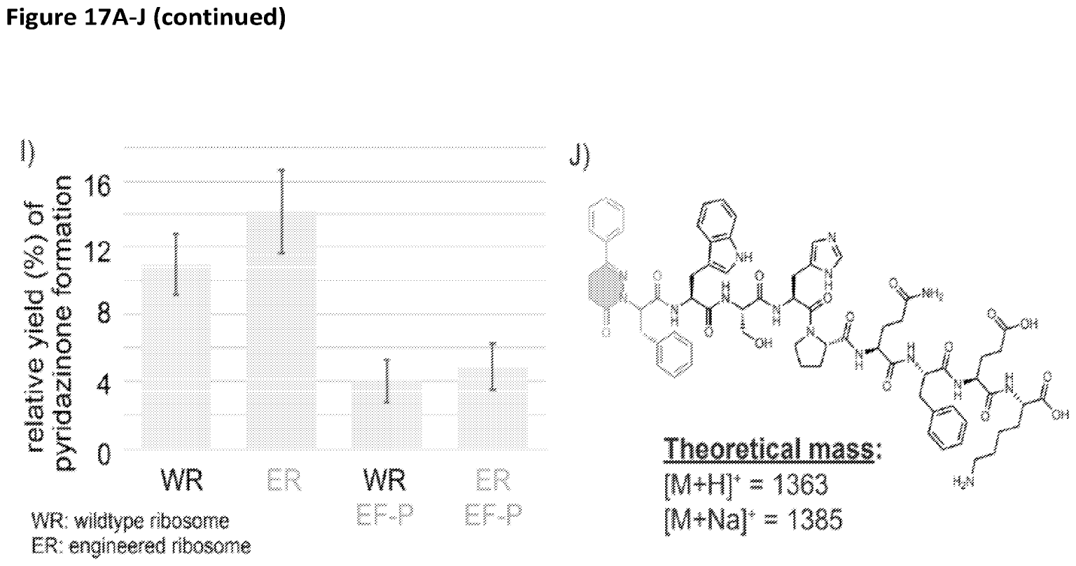

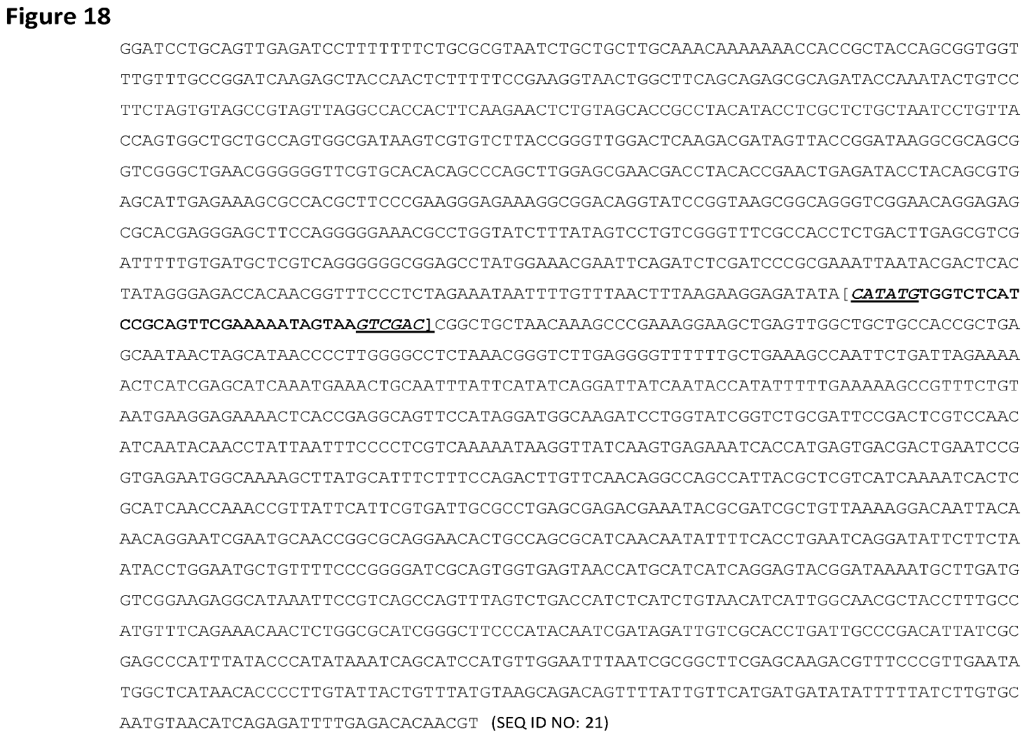

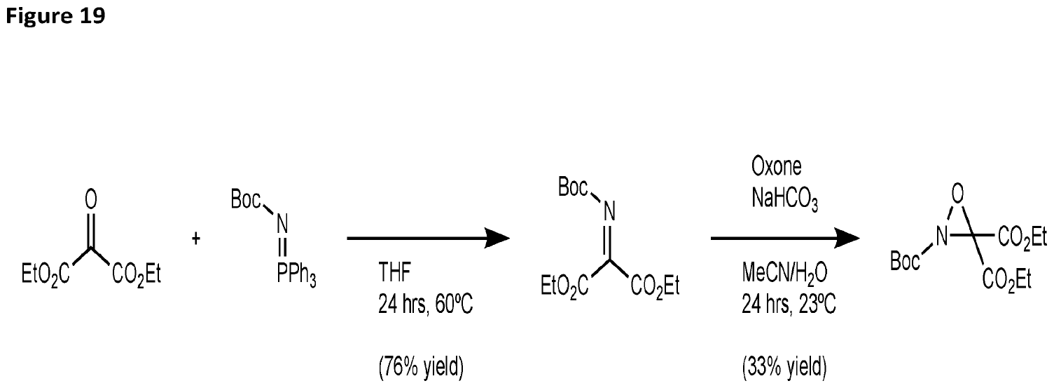

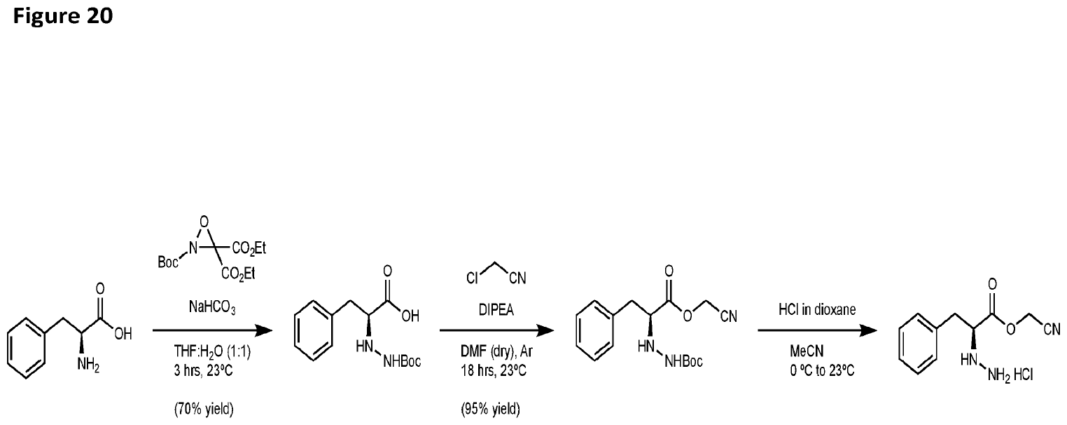

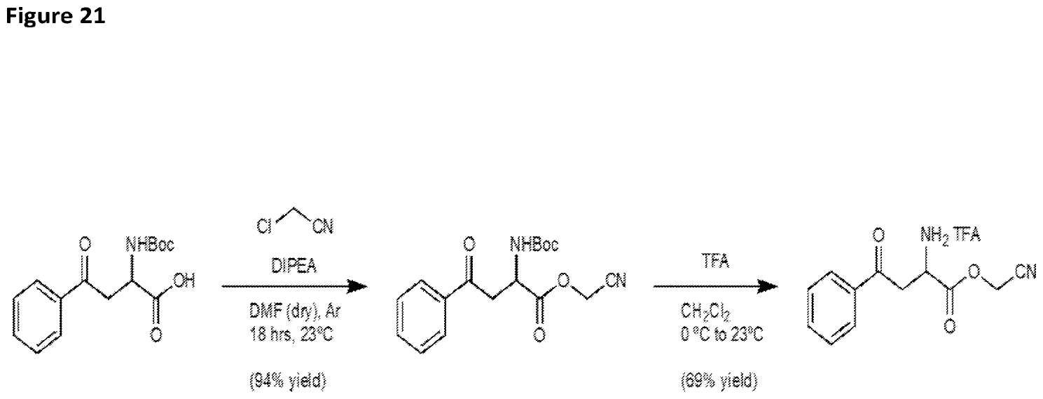

While wild type ribosomes are able to utilize the tRNAs charged with the novel oxo- and hydrazineyl acid substrates (see e.g., FIG. 3), the inventors discovered that an engineered ribosome, which can be utilized as a chemical machine, provided a higher rate of synthesis of the pyridazinone bonds to generate peptides and proteins.

The engineered ribosome was initially selected using beta3-puromycine. This engineered ribosome has the flexibility to incorporate other non-canonical substrates such as dipeptide, D-amino acids, and cyclic gamma amino acids, (see e.g., Maini, R. et al. Ribosome-mediated incorporation of dipeptides and dipeptide analogues into proteins in vitro. J. Am. Chem. Soc. 137, 11206-11209 (2015); Chen, S., Ji, X., Gao, M., Dedkova, L. M. & Hecht, S. M. In cellulo synthesis of proteins containing a fluorescent oxazole amino acid. J. Am. Chem. Soc. 141, 5597-5601 (2019); Maini, R. et al. Protein synthesis with ribosomes selected for the incorporation of beta-amino acids. Biochemistry 54, 3694-3706 (2015), incorporated herein by reference in their entireties).

### Engineered Translation Factors

While two translational factors; EF-P, and EF-Tu were used, the disclosed technology is not intended to be so limited. EF-P was not engineered. EF-Tu was engineered to have mutations at a specific site (see e.g., T. Katoh, I. Wohlgemuth, M. Nagano, M. V. Rodnina and H. Suga, Essential structural elements in tRNA(Pro) for EF-P-mediated alleviation of translation stalling, Nat. Commun., 2016, 7, 11657; Chem. Commun., 2020, 56, 5597-5600, doi.org/10.1039/DOCC07740B Chem. Commun., 2021, Accepted Manuscript; ACS Synth. Biol. 2019, 8, 2, 287-296 J Mol Evol. 2017; 84(2): 69-84.

### Products

The present systems and components allow for the production of polymers with more complicated non-canonical chemical substrates rather than chain-like polymers (peptides and polyesters).

In addition, the systems and components allow for the production of novel protease-resistant protein/peptidomimetic drugs that could produce novel therapeutics or medicines.

Moreover, in addition to the synthesis of a variety of keto- and hydrazineyl acids, the present disclosure provides for the synthesis of polymers with non-natural, non-α-amino acid monomers (NNAs) required to biosynthesize sequence defined polypyrazolone, polypyridazinone, polydiazepinone, nylons, spider silks, polyolefins, polyaramids, polyurethanes, polyketides, polycarbonates, conjugated polymers, gamma amino acid polypeptides, delta-amino acid, epsilon-amino acid polypeptides, zeta-amino acid polypeptides, oligosaccharides, and oligonucleotides, polyvinyls, polyfurans.

By way of example, and not by way of limitation, the present systems and components allow for the synthesis of compounds such as of 4-oxo-4-phenylbutanoic acid, 3-oxo-3-phenylpropanoic acid, 3-phenylpropiolic acid, 2-hydrazineyl-4-oxo-4-phenylbutanoic acid, (Z)-3-chloro-3-(4-hydrazineylphenyl)acrylic acid, 2-hydrazineyl-2-methyl-3-oxobutanoic acid, 4-(4-hydrazineylphenyl)-4-oxobutanoic acid, 3-amino-4-oxo-4-phenylbutanoic acid, 2-amino-4-oxo-4-phenylbutanoic acid, 4-(4-(methylthio)phenyl)-4-oxobutanoic acid, 4-oxopentanoic acid, 4-oxohexanoic acid, 3-oxobutanoic acid, 3-oxopentanoic acid, 3-oxo-3-phenylpropanoic acid, 5-oxohexanoic acid, with a leaving group of either cyanomethylester (CME), dinitrobenzylester (DNB), or amino-derivatized benzyl thioester (ABT), as well as the synthesis of enantiomerically pure (L- or D-) and racemic aminophenylalanine, aminoglycine, amionalanine, aminovaline, aminoisoleucine, aminotyrosine with a leaving group of CME, DNB, and ABT.

The disclosed systems and methods allow for the production of peptide-polymer hybrids by incorporating new functionality, formerly inaccessible to peptides by ribosomal synthesis or their post-translational modification reactions. In addition, the present systems, components and methods allow for the production of longer sequence-defined polymers with consecutive incorporations (number of monomers: >100).

The disclosed methods, systems, components, and composition may be utilized for preparing sequence defined polymers in vitro and/or in vivo. In some embodiments, the disclosed methods may be performed to prepare a sequence defined polymer in a cell free synthesis system, where the sequence defined polymer is prepared via translating an mRNA comprising a codon corresponding to an anticodon of the acylated tRNA molecule.

In the disclosed methods, the R group of the acylated tRNA molecule is incorporated in the sequence defined polymer during translation of the mRNA. In some embodiments of the disclosed methods, the R group of the acylated tRNA molecule is incorporated in the sequence defined polymer during translation of the mRNA at the start codon (AUG) of the mRNA. In other embodiments of the disclosed methods, the R group of the acylated tRNA molecule is incorporated in the sequence defined polymer during translation of the mRNA at a codon for threonine (e.g., ACC), a codon for isoleucine (e.g., AUC), a codon for alanine (e.g. GCC), or at methionine (AUG).

### Illustrative Embodiments, Uses, and Advantages

1. Use of the ribosome for the synthesis of polymers containing non-amide, non-ester bonds.
2. Use of in vitro cell-free protein synthesis platform for the synthesis of polymers containing non-amide, non-ester bonds.
3. Synthesis of a variety of keto- and hydrazineyl acids.
4. Charging the substrates onto synthetic tRNAs with ribozymes (flexizyme).
5. Formation of heterocyclic covalent bonds connecting the non-canonical monomers using the ribosome.

6. Synthesis of 4-oxo-4-phenylbutanoic acid, 3-oxo-3-phenylpropanoic acid, 3-phenylpropiolic acid, 2-hydrazineyl-4-oxo-4-phenylbutanoic acid, (Z)-3-chloro-3-(4-hydrazineylphenyl)acrylic acid, 2-hydrazineyl-2-methyl-3-oxobutanoic acid, 4-(4-hydrazineylphenyl)-4-oxobutanoic acid, 3-amino-4-oxo-4-phenylbutanoic acid, 2-amino-4-oxo-4-phenylbutanoic acid, 4-(4-(methylthio)phenyl)-4-oxobutanoic acid, 4-oxopentanoic acid, 4-oxohexanoic acid, 3-oxobutanoic acid, 3-oxopentanoic acid, 3-oxo-3-phenylpropanoic acid, 5-oxohexanoic acid, with a leaving group of either cyanomethylester (CME), dinitrobenzylester (DNB), or amino-derivatized benzyl thioester (ABT).
7. Synthesis of enantiomerically pure (L- or D-) and racemic aminophenylalanine, aminoglycine, amionalanine, aminovaline, aminoisoleucine, aminotyrosine with a leaving group of CME, DNB, and ABT.
8. Use of novel monomers and their variants for the synthesis of polymers with non-natural, non-α-amino acid monomers (NNAs) required to biosynthesize sequence defined polypyrazolone, polypyridazinone, polydiazepinone, nylons, spider silks, polyolefins, polyaramids, polyurethanes, polyketides, polycarbonates, conjugated polymers, gamma amino acid polypeptides, delta-amino acid, epsilon-amino acid polypeptides, zeta-amino acid polypeptides, oligosaccharides, and oligonucleotides, polyvinyls, polyfurans.
9. Expanding the range of monomer building blocks confined to the substrates with one nucleophile and one electrophile (e.g. amino acid, hydroxy acid, thioacid) into the substrates with more than two nucleophiles and electrophiles.
10. Producing polymers with more complicated non-canonical chemical substrates rather than chain-like polymers (peptides and polyesters).
11. Producing novel protease-resistant protein/peptidomimetic drugs that could produce novel therapeutics or medicines.
12. Reprogramming synthetic/orthogonal tRNAs with the non-canonical substrates by ribozyme
13. Producing peptide-polymer hybrid by incorporating new functionality inaccessible to peptides by ribosomal synthesis or their post-translational modification reactions
14. Providing a model that can be used for engineering the translation machineries.
15. Producing longer sequence-defined polymers with consecutive incorporations (number of monomers: >100).
16. Changing the current paradigm of polymer productions that occurs in solution to cell-free protein synthesis platforms.

### EXAMPLES

The following Examples are illustrative and are not intended to limit the scope of the claimed subject matter.

### Example 1—Expanding the Chemical Substrates for Genetic Code Reprogramming

### Background

While current studies have reported more than 200 non-canonical substrates are charged into tRNA and incorporated into a peptide by the Fx approach, and multiple strategies have been devised to synthesize tRNAs charged with non-canonical amino acid, there still exist limitations and gaps in the range of substrates.

The Fx (an artificial ribozyme with the ability to aminoacylate an arbitrary tRNA) system has seen widespread success over the last decade in which a wide range of chemical substrates (α-amino acids, β-amino acids, γ-amino acids, D-amino acids, nonstandard amino acids, N-protected (alkylated) amino acids, fluorescent amino acids, and hydroxy acids, aromatic, aliphatic, malonyl, and oligomeric amino acids) have been incorporated into ribosomal peptide chain through mis-acylated tRNAs and produced different types of polymers such as polyamides, polyesters, polythioesters, polythioamides.

However, the chemical bond synthesized so far other than amide-(peptide) has been confined to ester and thioester by the use of hydroxy and thioacids because the translational machinery has been evolutionarily optimized to form amide using the canonical 20 amino acid building blocks.

Here, we set out to produce new covalent chemical bonds by using rationally designed non-canonical monomer substrates that are charged to tRNA by ribozyme and form a new chemical bond by the ribosome-mediated protein translation process in a cellfree platform. We investigated to create heterocyclic chemical bond between keto-acid and hydrazineyl acid substrates and show the ribosome can be used as a chemical machine to build sequence-defined polymer based on the information read from mRNA.

Our rationally designed substrates and ribosome-mediated polymerization would produce polymers with novel functionality that are inaccessible by the posttranslational modifications, which we believe open up the possibility of creating next-generation based-commodities such as polymers and therapeutics that need to be precisely designed for high-tech science and personalized drugs. Our result can be leveraged as a foundational resource for chemists, biochemists, and molecular biologists as well as protein engineers to select a proper non-canonical substrate. Finally, our substrate variants set could be readily applied to chemical substrate variants for the synthesis of various peptides, including precursors for therapeutic medicines and macrocyclic materials. This novel and comprehensive study have advantages for fundamental and synthetic/engineering biology.

### Abstract

Ribosome-mediated polymerization is a powerful technology due to the ribosome's ability to polymerize monomers at a rapid rate (20 aa/sec) and high fidelity (99.999%). However, the type of polymers that can be produced by the ribosome has been mostly confined to polyamide analogues because natural ribosomes have been evolutionarily optimized to form a peptide (amide) bond between monomers. Here, we rationally design a variety of non-canonical chemical substrates that could form a non-amide polymer backbone when site specifically incorporated into a peptide and demonstrate that the ribosome enables the formation of 5-, 6-, and 7-membered heterocyclic structures such as pyrazolone, pyridazinone, and diazepinone. We optimize the bond formation reaction using an engineered ribosome and translation factors and show the engineered ribosome produced the bond more efficiently than the wild type ribosome. Moreover, we expand the range of non-canonical substrates into oxo- and hydrazineyl acid substrates and present a wide variety of heterocyclic ring derivatives are produced under the optimized reaction condition. We finally show consecutive incorporation of these monomers into a peptide, and produce various polymers containing multiple cyclic bonds. This suggests that our ribosome-mediated polymerization approach can be a transformative technology to produce alternating block copolymers such as AB, ABA, or ABAB.

### Results and Advantages

The heterocyclic chemical bonds we synthesized using the ribosome-mediated polymerization in vitro are frequently found in natural products and drugs that are used in pharmaceutical field (e.g. Metamizole; painkiller) because of their specific biological activities (e.g. protease-resistant) However, these products are only prepared by total synthesis or complicated biological pathways, which is slow, laborious, and expensive.

We used the ribosome that has been evolutionarily optimized to synthesize peptides and proteins as a chemical machine to produce these chemical bonds.

We reprogrammed tRNAs with non-canonical chemical substrates using a ribozyme that charges the substrates into 3′-hydroxyl group of synthetic tRNA

We added the reassigned tRNAs into a cell-free system, where the tRNAs are delivered to the ribosome by the translation factors. The non-canonical substrates react to each other and form heterocyclic products in the ribosome.

We showed that the wild type and engineered ribosomes form not only amide bond but also pyrazolone, pyridazinone, and diazepinone when oxo-acid and hydrazineyl acid are consecutively incorporated into a peptide on synthetic tRNAs.

We characterized the peptide with mass spectrometry and confirmed that the resulting peptide has a pyridazinone bond between the monomers.

This is the first work that shows the ability of the ribosome to produce novel sequence-defined polymer with an exotic covalent linkage between monomers, which could open up the possibility of producing a more diverse sequence-defined polymers (e.g., ABAB and ABAC type) bearing a more than one covalent linkage (e.g., carbon-carbon or carbon-nitrogen bond) between monomers in the ribosome.

### REFERENCES

1. Edelmann, P. & Gallant, J. Mistranslation in E. coli. Cell 10, 131-137 (1977).
2. Precup, J., Ulrich, A. K., Roopnarine, O. & Parker, J. Context specific misreading of phenylalanine codons. Mol Gen Genet 218, 397-401 (1989).
3. Rodnina, M. V. & Wintermeyer, W. Fidelity of aminoacyl-tRNA selection on the ribosome: kinetic and structural mechanisms. Annu Rev Biochem 70, 415-435 (2001).
4. Cropp, T. A., Anderson, J. C. & Chin, J. W. Reprogramming the amino-acid substrate specificity of orthogonal aminoacyl-tRNA synthetases to expand the genetic code of eukaryotic cells. Nature Protocols 2, 2590-2600 (2007).
5. Morimoto, J., Hayashi, Y., Iwasaki, K. & Suga, H. Flexizymes: their evolutionary history and the origin of catalytic function. Acc Chem Res 44, 1359-1368 (2011).
6. Albayrak, C. & Swartz, J. R. Cell-free co-production of an orthogonal transfer RNA activates efficient site-specific non-natural amino acid incorporation. Nucleic Acids Res 41, 5949-5963 (2013).
7. Chin, J. W. Expanding and reprogramming the genetic code. Nature 550, 53-60 (2017).
8. Mukai, T., Lajoie, M. J., Englert, M. & Soll, D. Rewriting the genetic code. Annu Rev Microbiol 71, 557-577 (2017).
9. Voller, J. S. & Budisa, N. Coupling genetic code expansion and metabolic engineering for synthetic cells. Curr Opin Biotech 48, 1-7 (2017).
10. Vargas-Rodriguez, O., Sevostyanova, A., Soll, D. & Crnkovic, A. Upgrading aminoacyltRNA synthetases for genetic code expansion. Curr Opin Chem Biol 46, 115-122 (2018).
11. Arranz-Gibertt, P., Vanderschurent, K. & Isaacs, F. J. Next-generation genetic code expansion. Curr Opin Chem Biol 46, 203-211 (2018).
12. Tajima, K., Katoh, T. & Suga, H. Genetic code expansion via integration of redundant amino acid assignment by finely tuning tRNA pools. Curr Opin Chem Biol 46, 212-218 (2018).
13. Rogers, J. M. & Suga, H. Discovering functional, non-proteinogenic amino acid containing, peptides using genetic code reprogramming. Org Biomol Chem 13, 9353-9363 (2015).
14. Obexer, R., Walport, L. J. & Suga, H. Exploring sequence space: harnessing chemical and biological diversity towards new peptide leads. Curr Opin Chem Biol 38, 52-61 (2017).
15. Fujino, T., Goto, Y., Suga, H. & Murakami, H. Ribosomal synthesis of peptides with multiple beta-amino acids. J Am Chem Soc 138, 1962-1969 (2016).
16. Ohshiro, Y. et al. Ribosomal synthesis of backbone-macrocyclic peptides containing gamma-amino acids. ChemBioChem 12, 1183-1187 (2011).
17. Goto, Y., Murakami, H. & Suga, H. Initiating translation with D-amino acids. RNA 14, 1390-1398 (2008).
18. Katoh, T., Tajima, K. & Suga, H. Consecutive elongation of D-amino acids in translation. Cell Chem Biol 24, 46-54 (2017).
19. Kawakami, T., Ishizawa, T. & Murakami, H. Extensive reprogramming of the genetic code for genetically encoded synthesis of highly N-alkylated polycyclic peptidomimetics. J Am Chem Soc 135, 12297-12304 (2013).
20. Iwane, Y. et al. Expanding the amino acid repertoire of ribosomal polypeptide synthesis via the artificial division of codon boxes. Nat Chem 8, 317-325 (2016).
21. Terasaka, N., Iwane, Y., Geiermann, A. S., Goto, Y. & Suga, H. Recent developments of engineered translational machineries for the incorporation of non-canonical amino acids into polypeptides. Int J Mot Sci 16, 6513-6531 (2015).
22. Ohta, A., Murakami, H., Higashimura, E. & Suga, H. Synthesis of polyester by means of genetic code reprogramming. Chem Biol 14, 1315-1322 (2007).19
23. Ohta, A., Murakami, H. & Suga, H. Polymerization of alpha-hydroxy acids by ribosomes. ChemBioChem 9, 2773-2778 (2008).
24. Goto, Y. & Suga, H. Translation initiation with initiator tRNA charged with exotic peptides. J Am Chem Soc 131, 5040-5041 (2009).
25. Rogers, J. M. et al. Ribosomal synthesis and folding of peptide-helical aromatic foldamer hybrids. Nat Chem 10, 405-412 (2018).
26. Torikai, K. & Suga, H. Ribosomal synthesis of an amphotericin-B inspired macrocycle. J Am Chem Soc 136, 17359-17361 (2014).
27. Kawakami, T., Ogawa, K., Hatta, T., Goshima, N. & Natsume, T. Directed evolution of a cyclized peptoid-peptide chimera against a cell-free expressed protein and proteomic profiling of the interacting proteins to create a protein-protein interaction inhibitor. ACS Chem Biol 11, 1569-1577 (2016).
28. Kanter, G. et al. Cell-free production of scFv fusion proteins: an efficient approach for personalized lymphoma vaccines. Blood 109, 3393-3399 (2007).
29. Cho, H. et al. Optimized clinical performance of growth hormone with an expanded genetic code. Proc Natl Acad Sci USA 108, 9060-9065 (2011).
30. Axup, J. Y. et al. Synthesis of site-specific antibody-drug conjugates using unnatural amino acids. Proc Natl Acad Sci USA 109, 16101-16106 (2012).
31. Zimmerman, E. S. et al. Production of site-specific antibody-drug conjugates using optimized non-natural amino acids in a cell-free expression system. Bioconjug Chem 25, 351-361 (2014).
32. Raucher, D. & Ryu, J. S. Cell-penetrating peptides: strategies for anticancer treatment. Trends Mol Med 21, 560-570 (2015).
33. Despanie, J., Dhandhukia, J. P., Hamm-Alvarez, S. F. & MacKay, J. A. Elastin-like polypeptides: Therapeutic applications for an emerging class of nanomedicines. J Control Release 240, 93-108 (2016).
34. Martin, R. W. et al. Development of a CHO-based cell-free platform for synthesis of active monoclonal antibodies. ACS Synth Biol 6, 1370-1379 (2017).
35. Heckler, T. G. et al. T4 RNA ligase mediated preparation of novel “chemically misacylated” tRNAPheS. Biochemistry 23, 1468-1473 (1984).
36. Robertson, S. A., Noren, C. J., Anthony-Cahill, S. J., Griffith, M. C. & Schultz, P. G. The use of 5′-phospho-2 deoxyribocytidylylriboadenosine as a facile route to chemical aminoacylation of tRNA. Nucleic Acids Res 17, 9649-9660 (1989).
37. Robertson, S. A., Ellman, J. A. & Schultz, P. G. A general and efficient route for chemical aminoacylation of transfer RNAs. J Am Chem Soc 113, 2722-2729 (1991).
38. Kwiatkowski, M., Wang, J. F. & Forster, A. C. Facile synthesis of N-acyl-aminoacyl-pCpA for preparation of mischarged fully ribo tRNA. Bioconjug Chem 25, 2086-2091 (2014).
39. Wang, J. F., Kwiatkowski, M. & Forster, A. C. Ribosomal peptide syntheses from activated substrates reveal rate limitation by an unexpected step at the peptidyl site. J Am Chem Soc 138, 15587-15595 (2016).
40. Yamanaka, K., Nakata, H., Hohsaka, T. & Sisido, M. Efficient synthesis of non-natural mutants in Escherichia coli S30 in vitro protein synthesizing system. J Biosci Bioeng 97, 395-399 (2004).
41. Liu, D. R. & Schultz, P. G. Progress toward the evolution of an organism with an expanded genetic code. Proc Natl Acad Sci USA 96, 4780-4785 (1999).
42. Wang, L., Brock, A., Herberich, B. & Schultz, P. G. Expanding the genetic code of Escherichia coli. Science 292, 498-500 (2001).
43. Nozawa, K. et al. Pyrrolysyl-tRNA synthetase-tRNA(Pyl) structure reveals the molecular basis of orthogonality. Nature 457, 1163-1167 (2009).20
44. Hancock, S. M., Uprety, R., Deiters, A. & Chin, J. W. Expanding the genetic code of yeast for incorporation of diverse unnatural amino acids via a pyrrolysyl-tRNA synthetase/tRNA pair. J Am Chem Soc 132, 14819-14824 (2010).
45. Neumann, H., Slusarczyk, A. L. & Chin, J. W. De novo generation of mutually orthogonal aminoacyl-tRNA synthetase/tRNA pairs. J Am Chem Soc 132, 2142-2144 (2010).
46. Chin, J. W. Expanding and reprogramming the genetic code of cells and animals. Annu Rev Biochem 83, 379-408 (2014).
47. Ellefson, J. W. et al. Directed evolution of genetic parts and circuits by compartmentalized partnered replication. Nat Biotechnol 32, 97-101 (2014).
48. Schmied, W. H., Elsasser, S. J., Uttamapinant, C. & Chin, J. W. Efficient multisite unnatural amino acid incorporation in mammalian cells via optimized pyrrolysyl tRNA synthetase/tRNA expression and engineered eRF1. J Am Chem Soc 136, 15577-15583 (2014).
49. Amiram, M. et al. Evolution of translation machinery in recoded bacteria enables multi-site incorporation of nonstandard amino acids. Nat Biotechnol 33, 1272-1279 (2015).
50. Willis, J. C. W. & Chin, J. W. Mutually orthogonal pyrrolysyl-tRNA synthetase/tRNA pairs. Nat Chem 10, 831-837 (2018).
51. Saito, H. & Suga, H. A ribozyme exclusively aminoacylates the 3′-hydroxyl group of the tRNA terminal adenosine. J Am Chem Soc 123, 7178-7179 (2001).
52. Lee, N., Bessho, Y., Wei, K., Szostak, J. W. & Suga, H. Ribozyme-catalyzed tRNA aminoacylation. Nat Struct Biol 7, 28-33 (2000).
53. Murakami, H., Saito, H. & Suga, H. A versatile tRNA aminoacylation catalyst based on RNA. Chem Bio110, 655-662 (2003).
54. Ramaswamy, K., Saito, H., Murakami, H., Shiba, K. & Suga, H. Designer ribozymes: programming the tRNA specificity into flexizyme. J Am Chem Soc 126, 11454-11455 (2004).
55. Murakami, H., Ohta, A., Ashigai, H. & Suga, H. A highly flexible tRNA acylation method for non-natural polypeptide synthesis. Nat Methods 3, 357-359 (2006).
56. Xiao, H., Murakami, H., Suga, H. & Ferre-D'Amare, A. R. Structural basis of specific tRNA aminoacylation by a small in vitro selected ribozyme. Nature 454, 358-361 (2008).
57. Passioura, T. & Suga, H. Flexizyme-mediated genetic reprogramming as a tool for noncanonical peptide synthesis and drug discovery. Chemistry 19, 6530-6536 (2013).
58. Niwa, N., Yamagishi, Y., Murakami, H. & Suga, H. A flexizyme that selectively charges amino acids activated by a water-friendly leaving group. Bioorg Med Chem Lett 19, 3892-3894 (2009).
59. Saito, H., Watanabe, K. & Suga, H. Concurrent molecular recognition of the amino acid and tRNA by a ribozyme. RNA 7, 1867-1878 (2001).
60. Goto, Y. et al. Reprogramming the translation initiation for the synthesis of physiologically stable cyclic peptides. ACS Chem Biol 3, 120-129 (2008).
61. Saito, H., Kourouklis, D. & Suga, H. An in vitro evolved precursor tRNA with aminoacylation activity. EMBO J20, 1797-1806 (2001).
62. Goto, Y., Katoh, T. & Suga, H. Flexizymes for genetic code reprogramming. Nat Protoc 6, 779-790 (2011).
63. Das, R. & Baker, D. Macromolecular modeling with Rosetta. Annu Rev Biochem 77, 363-382 (2008).
64. Carlson, E. D., Gan, R., Hodgman, C. E. & Jewett, M. C. Cell-free protein synthesis: applications come of age. Biotechnol Adv 30, 1185-1194 (2012).
65. Kwon, Y. C. & Jewett, M. C. High-throughput preparation methods of crude extract for robust cell-free protein synthesis. Sci Rep 5, 8663 (2015).21
66. Jaroentomeechai, T. et al. Single-pot glycoprotein biosynthesis using a cell-free transcription-translation system enriched with glycosylation machinery. Nat Commun 9 (2018).
67. Kightlinger, W. et al. Design of glycosylation sites by rapid synthesis and analysis of glycosyltransferases. Nat Chem Biol 14, 627-635 (2018).
68. Shimizu, Y. et al. Cell-free translation reconstituted with purified components. Nat Biotechnol 19, 751-755 (2001).
69. Iwane, Y., Katoh, T., Goto, Y. & Suga, H. Artificial division of codon boxes for expansion of the amino acid repertoire of ribosomal polypeptide synthesis. Methods Mol Biol 1728, 17-47 (2018).
70. Udagawa, T., Shimizu, Y. & Ueda, T. Evidence for the translation initiation of leaderless mRNAs by the intact 70 S ribosome without its dissociation into subunits in eubacteria. J Biol Chem 279, 8539-8546 (2004).
71. Oza, J. P. et al. Robust production of recombinant phosphoproteins using cell-free protein synthesis. Nat Commun 6 (2015).
72. Liu, Y., Kim, D. S. & Jewett, M. C. Repurposing ribosomes for synthetic biology. Curr Opin Chem Biol 40, 87-94 (2017).
73. d'Aquino, A. E., Kim, D. S. & Jewett, M. C. Engineered ribosomes for basic science and synthetic biology. Annu Rev Chem Biomol Eng 9, 311-340 (2018).

U.S. Pat. No. 5,478,730 16 Dec. 1992 26 Dec. 1995 Institute of Protein Research Method of preparing polypeptides in cell-free translation system

U.S. Pat. No. 5,556,769 23 Aug. 1994 17 Sep. 1996 Coupled replication-translation methods and kits for protein synthesis

U.S. Pat. No. 5,665,563 13 Feb. 1995 9 Sep. 1997 Promega Corporation Coupled transcription and translation in eukaryotic cell-free extract

U.S. Pat. No. 6,168,931 17 Mar. 1999 2 Jan. 2001 The Board of Trustees of the Leland Stanford Junior University Enhanced in vitro synthesis of biological macromolecules using a novel ATP regeneration system

U.S. Pat. No. 6,518,058 23 Apr 2001 11 Feb. 2003 Roche Diagnostics GmbH Method of preparing polypeptides in cell-free system and device for its realization

U.S. Pat. No. 6,783,957 21 Jan. 2003 31 Aug. 2004 Roche Diagnostics GmbH Method for synthesis of polypeptides in cell-free systems

U.S. Pat. No. 6,869,774 28 Aug. 2001 22 Mar. 2005 Yaeta Endo Methods of synthesizing cell-free protein

U.S. Pat. No. 6,994,986 7Sep. 2001 7Feb. 2006 The Board of Trustees of the Leland Stanford University In vitro synthesis of polypeptides by optimizing amino acid metabolism

U.S. Pat. No. 7,118,883 23 Oct. 2001 10 Oct. 2006 Post Genome Institute Co., Ltd. Process for producing peptides by using in vitro transcription/translation system

U.S. Pat. No. 7,189,528 10 Dec. 2002 13 Mar. 2007 Shimadzu Corporation Extract solution for cell-free protein synthesis, method for cell-free protein synthesis using same and method for preparation of the extract solution

U.S. Pat. No. 7,338,789 18 Aug. 2003 4 Mar. 2008 The Board of Trustees of the Leland Stanford Junior University Methods of in vitro protein synthesis

U.S. Pat. No. 7,387,884 7 Jan. 2004 17Jun. 2008 Shimadzu Corporation Yeast extract solution for cell free protein synthesis, method for preparation thereof and method for cell-free protein synthesis using same

U.S. Pat. No. 7,399,610 12 Oct. 2005 15 Jul. 2008 Shimadzu Corporation/Institute of Protein Research Method for cell-free protein synthesis using extract solution derived from insect cell

US20090281280 5 Dec. 2006 12 Nov. 2009 University of Tokyo Versatile tRNA Acylation Catalytic RNAs and Uses Thereof

WO2008059823 13 Nov. 2007 22May 2008 University of Tokyo Translation and synthesis of polypeptide having nonnative structure at n-terminus and application thereof

EP2141175 26 Mar. 2008 6 Jan. 2010 University of Tokyo Process for synthesizing cyclic peptide compound

WO2011049157 21 Oct. 2010 28 Apr. 2011 PeptiIDream Inc. Rapid display method in translational synthesis of peptide

WO2012026566 26 Aug. 2011 1 Mar. 2012 University of Tokyo Novel artificial translation/synthesis system

U.S. Pat. No. 9,410,148 8 Sep. 2011 9 Aug. 2016 University of Tokyo Method for constructing libraries of non-standard peptide compounds comprising N-methyl amino acids and other special (non-standard) amino acids and method for searching and identifying active

JP2013071904 27 Sep. 2011 22 Apr. 2013 PeptilDream Inc. Peptide having anti-influenza virus activity

WO2012074129 5 Dec. 2011 7 Jun. 2012 University of Tokyo Peptide with safer secondary structure, peptide library, and production methods for same

WO2012074130 5 Dec. 2011 7 Jun. 2012 University of Tokyo Peptide library production method, peptide library, and screening method

WO2013100132 28 Dec. 2012 4 Jul. 2013 Chugai Pharmaceutical Co. Peptide-compound cyclization method

WO2014119600 29 Jan. 2014 7 Aug. 2014 PeptilDream Inc. Flexible display method

US20160289668 4 Aug. 2014 6 Oct. 2016 PeptiIDream Inc. University of Tokyo Production Method for Charged Non-Protein Amino Acid-Containing Peptide

US20160209421 26 Aug. 2014 21 Jul. 2016 University of Tokyo Macrocyclic Peptide, Method for Producing Same, and Screening Method Using Macrocyclic Peptide Library

U.S. Pat. No. 9,783,800 3 Feb. 2015 10 Oct. 2017 University of Tokyo Method for producing peptides having azole-derived skeleton

JP2018509172 29 Mar. 2016 5 Apr. 2018 University of Queensland Platform for the non-natural amino acid incorporation into protein

WO2016199801 8 Jun. 2016 15 Dec. 2016 University of Tokyo Amino acid-modified nucleic acid and utilization thereof

JP2017216961 9 Jun. 2016 14 Dec. 2017 Saitama University Non-natural amino acid containing peptide library

EP2966174B1 7 Mar. 2013 21 Feb. 2018 University of Tokyo Method for producing compound containing heterocycle

EP3591048A1 3 Jan. 2017 8 Jan. 2020 Chugai Seiyaku Kabushiki Kaisha (Chugai Pharmaceutical co. ltd.) Method for synthesizing peptides in cell-free translation system

WO2007066627A1 6 Dec. 2005 14 Jun. 2007 University of Tokyo Multi-purpose acylation catalyst and use thereof

### Example 2: Ribosome-Catalyzed Formation of Pyridazinone Bonds In Vitro

### Summary

The ribosome is a macromolecular machine that catalyzes the sequence-defined polymerization of L-α-amino acids into peptides and proteins.¹ The extraordinary biosynthesis capability of the ribosome has long motivated efforts to understand and harness it for biotechnology.²⁻⁴ For example, reprogramming the genetic code to incorporate non-canonical amino acids into proteins has led to new classes of medicines and materials.⁵⁻⁷ While the ribosome has been used to incorporate numerous non-canonical amino acids into peptides and proteins,⁸ it has evolved to perform a single type of chemistry—chain-growth condensation polymerization via peptide bond formation. Here, we demonstrate ribosome-mediated polymerization of pyridazinone bonds, rather than peptide bonds, via the cyclocondensation reaction between γ-keto and α-hydrazino ester monomers. We first designed and synthesized a repertoire of monomers and assessed their ability to be acylated on to transfer RNAs (tRNAs). Then, we showed that the resulting tRNA-monomers could be used by ribosomes in in vitro translation to form pyridazinone bonds. Finally, we demonstrate the ribosome-catalyzed synthesis of peptide-hybrid oligomers composed of multiple sequence-defined alternating pyridazinone linkages. Our results expand the range of non-canonical polymeric backbones that can be synthesized by the ribosome and open the door to new applications in synthetic biology.

### Discussion

Guided by messenger RNA (mRNA) templates and the genetic code, the ribosome is the catalytic workhorse of the translation apparatus, polymerizing the successive condensation of amino acid monomers into sequence defined polymers. In nature, with rare exceptions, these polymers are composed of 20 canonical amino acids. However, genetic code reprogramming technologies can site-specifically incorporate non-canonical amino acids (ncAAs) into proteins to expand the range of genetically encoded chemistry.⁸⁻¹¹ To date, hundreds of ncAAs have been co-translationally incorporated into proteins;⁸ including L-α- (e.g., p-azido-phenylalanine), β-, γ-, δ-, ε-, ζ-, cyclic, and N-alkylated amino acids, among others.¹²⁻¹⁷ Site-specific incorporation of such ncAAs into peptides and proteins, as well as alternative monomers (e.g., non-amino carboxylic acids, hydroxy acids, aminoxy acids, hydrazino acids, and thioacids),¹⁸⁻²² have transformed the way that we study protein and cellular function and enabled synthetic biology applications.

While genetic code expansion has extended the limits of monomers amenable to ribosome-mediated polymerization, their polymeric structures remain confined to a much smaller chemical space composed of peptide bonds (amide linkages, (—CONH—),^{15-17, 23, 24}), or close analogs like esters (—COO—),^{20, 25} thioesters (—COS—),²⁶ or thioamides (—CSNH—).²² This is because wild-type ribosomes have evolved over billions of years to prefer L-α-amino acid substrates and to polymerize via peptide (i.e., amide) bond formation.^{27, 28} Expanding the repertoire of bond formation chemistries made by the ribosome will help elucidate constraints on the chemistry that the ribosome's RNA-based active site can achieve and enable bio-derived polymeric backbones that go beyond natural limits. However, the peptidyl transferase mechanism, wherein a nucleophilic α-amino group of A-site aminoacyl-tRNA consecutively attacks an electrophilic ester linkage of the P-site tRNA carrying the growing polymeric (i.e., peptide or protein) chain, has limited such efforts.

To address this limitation, the inventors developed alternative polymer backbone chemistries suitable for ribosome-mediated polymerization. Given the high structural dependance of peptide bond formation in the evolutionary optimized peptidyl transferase center, the inventors hypothesized that any new ribosomal monomer would need to closely resemble the structure of the proteinogenic amino acids, such that the reactive components would be oriented correctly for a bond reaction to occur. Considering the structures of natural amino acids, the inventors chose to use monomers that possess a nucleophilic hydrazine group in place of the α-amine. The inventors hypothesized that hydrazino acids could provide two reactive sites (i.e., the α- or β-nitrogen) to facilitate ribosome-mediated ligation with γ-keto esters (FIG. 7), producing 6-membered heterocyclic rings called pyridazinones.²⁹ Several features supported this design choice. First, the heterocyclic literature is replete with reactions between hydrazines and keto esters to form pyrazolones, pyridazinones, and other heterocycles,³⁰⁻³² as these structural motifs are often found as key pharmacophores.^{33, 34} Second, several recent efforts have shown the ability to incorporate α-hydrazino monomers into peptides by the ribosome in vino.^{24, 35} Third, cyclocondensation of a hydrazine and a keto ester begins with hydrazone formation followed by cyclization, which is an amide forming step, a specialty of the ribosome.

Ribosome-catalyzed formation of pyridazinone bonds required the activated γ-keto and hydrazino ester monomers and the subsequent charging of these monomers to transfer RNAs (tRNAs). Since these monomers do not have associated aminoacyl-tRNA synthetases necessary for tRNA acylation, we decided to charge tRNAs with the flexizyme (Fx) system. Flexizymes are aminoacyl-tRNA synthetase-like ribozymes that catalyze the acylation of tRNA with diverse substrates.^{36, 37} Because Fx only recognizes the 3′-CCA sequence of tRNA and the benzyl group of an acyl substrate, virtually any monomer can be acylated so long as it possesses an appropriate activating group (e.g., cyanomethyl ester (CME), dinitrobenzylester (DNB), or (2-aminoethyl)amidocarboxybenzyl thioester (ABT)). Thus, Fx has been used extensively to expand the limits of a reprogrammed genetic code.^{18, 38}

We first designed a series of γ-keto and hydrazino monomers with different Fx-leaving groups to assess tRNA acylation (FIG. 8). The γ-keto ester substrates were prepared by esterification of γ-keto carboxylate with an activating group (AG).¹⁸ The hydrazino substrates were synthesized in three steps: (i) N-amination of Phe or Ala with an N-Boc-protected electrophilic amino source^{39, 40}, (ii) esterification of carboxylate with an AG⁴¹, and (iii) Boc deprotection from the β-nitrogen.¹⁶ To determine the acylation efficiency, we used a small tRNA mimic, microhelix (mihx, 22nt) as an acyl acceptor.³⁷ Fx-mediated reactions were carried out in 6 different conditions [2 different pHs (7.5 and 8.8) with three different flexizymes (eFx, dFx, and aFx)] for each synthetic γ-keto and α-hydrazino ester to optimize yields. Yields of Fx-catalyzed acylation were determined by a densitometric analysis of RNA bands on an acidic polyacrylamide gel (pH 5.2, 3 mM NaOAc), and ranged from 21-82% (FIG. 11).

Using the conditions optimized from our Fx-mihx experiments, we produced acyl-tRNAs bearing four γ-keto ester and two hydrazine monomers (FIG. 8A & 8B). After the Fx-mediated tRNA acylation, unreacted monomers were separated from the tRNAs using ethanol precipitation. The resulting tRNA fraction that includes the tRNA-substrates was supplemented as a mixture into an in vitro transcription and translation reaction containing a minimal set of components required for translation (PURExpress™).⁴² As a reporter oligomer, we designed a T7 promoter-controlled plasmid (pJL1_StrepII) encoding a Streptavidin tag (XY+WSHPQFEK (SEQ ID NO: 7)), where X and Y indicate the positions to which a Fx-charged γ-ketoester (1) and hydrazino substrate (5) are incorporated, respectively. The in vitro transcription and translation reactions were carried out in the presence of all E. coli (>46) endogenous tRNAs, but only eight amino acids encoding the polypeptide Streptavidin tag. For site-specific incorporation of 1 and 5 into the N-terminal X and Y residue, we first charged the substrate 1 and 5 onto tRNA^{fMet}(CAU) and tRNA^{Pro1E2}(GGU), respectively.^{43, 44} We selected the AUG and ACC codons on mRNA because the AUG (CAU anticodon) codon is the canonical initiation codon for N-terminal incorporation, and Thr (ACC) is excluded from the polypeptide Streptavidin tag. This prevented corresponding endogenous tRNAs in the PURExpress™ reaction from being aminoacylated, and from competing in the translation reaction. For the incorporation of 5, tRNA^{Pro1E2}(GGU)^{16, 43} was selected because it has an engineered D-arm and T-stem for interacting with translation elongation factors to promote the incorporation of a charged substrate.^{16, 45, 46}

Following in vitro translation in PURExpress™ reactions for 2 hours, the synthesized oligomers were denatured with SDS, and characterized by matrix-assisted laser desorption/ionization-time-of-flight (MALDI-TOF) mass spectrometry. We observed a peak corresponding to the mass of oligomer bearing a pyridazinone bond between 1 and 5 incorporated consecutively into the oligomer. The percent yield of pyridazinone formation of ˜10% was calculated based on the relative peak area of the peptides shown in the mass spectrum from 1000 to 2000 Da (FIG. 8C & 8D). While inefficient, this is to our knowledge the first example of intramolecular cyclic structure formation catalyzed by the ribosome in vitro.

We next tried to enhance the yield of oligomers containing the pyridazinone. First, we incubated the PURExpress™ reaction mixture for a longer time (24 h). Unfortunately, extending the reaction time did not increase production of pyridazinone-peptide product (FIG. 12-13). Second, we changed the hydrazine monomer. Previous studies have shown that bulky amino acid substrates have reduced incorporation efficiencies and product formation.¹⁷ Thus, instead of cyanomethyl amino-L-phenylalaninate (S)-HzPhe 5, we used the less bulky 3,5-dinitrobenzyl amino-L-alaninate (S)-HzAla 6, which contains only a methyl group on the side chain, to see if pyridazinone oligomer formation could be increased. As above, we carried out Fx-mediated tRNA acylation, isolated tRNA complexes, supplemented them into the PURExpress™ reaction, and characterized the resulting product by MALDI-TOF. In the MALDI spectrum (FIG. 8E), we observed a peak corresponding to the mass of the oligomer containing a pyridazinone in a yield of 48.3% of the total product. This ˜4-fold increase in yield indicates the natural translation system can incorporate less bulky (S)-HzAla 6 at higher efficiencies compared to (S)-HzPhe 5.

To further explore the ribosome-mediated pyridazinone formation reaction, we next tested additional γ-keto acids with both hydrazino esters (5 and 6). Specifically, we used cyanomethyl 4-(4-(methylthio)phenyl)-4-oxobutanoate (2, γKPheSMe-CME), 3,5-dinitrobenzyl 4-oxopentanoate (3, γKMe-DNB), and 3,5-dinitrobenzyl 4-oxohexanoate (4, γKEt-DNB). We carried out the Fx-mediated acylation reaction onto a tRNA^{fMet}(CUA) and tRNA^{Pro1E2}(GGU). Subsequently, we added the two tRNAs charged with a γ-keto and hydrazino ester in all the six possible combinations (i.e., 2:5, 3:5, 4:5, 2:6, 3:6, and 4:6) to PURExpress™ reactions. The MALDI-TOF spectra (FIG. 12-14) for each of the purified peptides show a peak corresponding to the theoretical mass of the oligomer containing a different pyridazinone.

Our data showed the ability of ribosome-mediated cyclocondensation to form eight different pyridazinone derivatives (FIG. 8C). However, we were concerned about the possibility that pyridazinone bonds could be created in the in vitro reaction without the ribosome between the hydrazino and keto ester monomers. We therefore performed a negative control reaction to assess possible pyridazinone formation with a PURExpress™ reaction under the same conditions as above with aminocyl-tRNA monomers 1 and 5 but without ribosomes (FIG. 9A). Following PURExpress™ reactions, we analyzed the crude reaction mixture by liquid chromatography-time-of-flight (LC-TOF) mass spectrometry. The extracted ion chromatogram deconvoluted based on the theoretical mass of 4-oxo-4-phenylbutanoic acid (OPA) and aminophenylalanine (APA) yielded a single peak corresponding to the theoretical masses of the expected monomers 1 (FIG. 9B) and 5 (FIG. 9C), respectively (inset). In contrast, no peak corresponding to the theoretical mass of the resulting pyridazinone (2-(6-oxo-3-phenyl-5,6-dihydropyridazin-1(4H)-yl)-3-phenylpropanoic acid, OPDP) was found (FIG. 9D). This result indicates that pyridazinone formation does not occur under our in vitro reaction conditions in the absence of the ribosome.

We next investigated the regioselectivity of the pyridazinone linkage. Two possible regioisomers may be produced in the peptidyltransferase center of the ribosome, a 1,6- and/or 2,6-substituted pyridazinone (FIG. 15). To better understand the regioselectivity of pyridazinone formation, we carried out chemical reactions between the γ-keto cyanomethyl ester (cyanomethyl 4-oxo-4-phenylbutanoate (OPBA, analogue to 1)) monomer with phenylhydrazine (APA, analogue to 5). The goal was to identify the regioselectivity of the resulting pyridazinone bonds produced in solution, by which we may infer the structure of pyridazinone produced in the ribosome. We carried out the reaction of 1 and 5 in three different concentrations (40 μM, 4 mM, and 40 mM) in MeOH/H₂O (3/2:v/v) at 37° C. and analyzed the resulting product by liquid chromatography-mass spectrometry (LC-MS) at two different reaction times (2 h and 24 h). The pyridazinone product was not found by LC-MS at the concentrations of 40 μM and 4 mM, while the peaks for the carboxylic acid (hydrolyzed by water), the starting cyanomethyl ester, and the methyl ester (esterified by methanol) were observed at 4.2, 5.0, and 5.4 min, respectively (FIG. 16A & 16B). Importantly, 40 μM is the concentration of tRNA^{fMet}(CAU):1 and tRNA^{Pro1E2}(GGU):5 supplemented into the PURExpress™ reactions that catalyzed pyridazinone formation, further confirming that the ribosome is necessary for production of the pyridazinone-peptide hybrids. In the 40 mM reaction, a new peak was observed at 7.7 min (FIG. 16C, yield: 2% (2 h) and 13% (24 h)), which corresponds to the theoretical mass of OPDP. Analysis by ¹H NMR spectroscopy (FIG. 16D) showed that the isolated product was exclusively the 2,6-substituted pyridazinone. Admittedly, the reactivity of the α- and β-nitrogen in the ribosome might be different, leading to the formation of an amide and hydrazone bond linked to either the α- and β-nitrogen. Further investigations involving new synthetic substrates that selectively form an amide with α- or β-nitrogen atom might be a possible strategy to elucidate the structure of the ribosome-generated pyridazinone more clearly.

After confirming the ribosome is a necessary catalyst for pyridazinone ring formation in our PURExpress™ reaction conditions, we explored the impact of supplementing additional translation factors. Previously, supplementing in vitro transcription and translation reactions with engineered ribosomes^{47, 48} and Elongation Factor P (EF-P) have increased yields of polymers with poorly compatible substrates.¹⁵⁻¹⁸ For example, the Hecht group showed that an engineered ribosome, termed 040329, enabled incorporation of dipeptides by the ribosome, which was later shown to facilitate incorporation of backbone extended monomers.¹⁵ EF-P is a bacterial translation factor that accelerates peptide bond formation between consecutive prolines and has been shown to help alleviate ribosome stalling as a result of D- and β- amino acid substrates.^{16, 45} To test if supplementation benefitted synthesis of pyridazinone-peptide oligomers, we prepared purified mutant ribosomes as a mixture of wild type and 040329 ribosomes and EF-P, as done before (Supplementary Information).¹⁵ We carried out PURExpress™ reactions with substrates 1 and 5, and purified and analyzed the products by MALDI-TOF mass spectrometry. In the resulting MALDI spectra, we observed the peak corresponding to the theoretical mass of a target oligomer containing a pyridazinone bond increases ˜3% in the presence of engineered ribosomes (FIG. 17). However, production of a pyridazinone bond was inhibited ˜8%, when EF-P was supplemented with just wild-type ribosomes or combined wild-type or engineered ribosomes.

To test the limits of the sequence-defined incorporation of pyridazinone linkages in vitro, we sought to program the production of multiple alternating oligopyridazinones. To do so, we leveraged our previous design rules for Fx-mediated site-specific incorporation¹⁸ and synthesized cyanomethyl 2-amino-4-oxo-4-phenylbutanoate (7, FIG. 10). After C-terminal extension of the Strep-tag with 7, we envisioned that the reactive γ-keto handle could undergo a cyclocondensation reaction with the α-hydrazino acid 6 programed at the subsequent codon to form a pyridazinone linkage. Further extensions would be accomplished by sequential incorporation of 7 followed by 6 (FIG. 10).

For demonstration purposes, we designed additional plasmids (pJL1-StrepII-TI2 and pJL1-StrepII-TI3) that allow the incorporation of (S)-γ-keto amino acid 7 and (S)-HzAla 6 repeatedly in an alternating fashion at the C-terminus. We envisioned these monomers would produce peptides containing two or three consecutive pyridazinones, when four or six multiple incorporations are created by the ribosome, respectively. We used HzAla 6 instead of HzPhe 5 for the multiple pyridazinone bond formation, because the introduction of the substrates with a bulky side chain might limit the ribosome's polymerization capability as shown in FIG. 8C. For efficient multiple incorporations, we also supplemented 3-4 times higher amounts of tRNA^{Pro1E2}(GGU):7 and tRNA^{GluE}2(GAU):6 complexes than the amount used for the single pyridazinone formation reaction. After carrying out in vitro transcription and translation reactions with the PURExpress™ system, we purified the resulting oligomers, and analyzed them by MALDI-TOF mass spectrometry. In our MALDI mass spectra (FIG. 10), we observed a peaks demonstrating ribosome-catalyzed synthesis of peptide-hybrid oligomers composed of multiple alternating pyridazinone linkages.

In this work we demonstrate ribosome catalyzed formation of pyridazinone linkages in vitro for the biosynthesis of pyridazinone-peptide hybrids. Our results have revealed several key features relevant to the development of new ribosome-catalyzed chain concatenations. First, while the field of genetic code reprogramming has reported hundreds of non-canonical chemical substrates, it was previously unclear if the ribosome could polymerize non-peptide backbone structures based on γ-keto and hydrazino ester monomers. We show that this is possible using a set of rationally designed monomers to synthesize pyridazinone bonds. Second, we verify our findings by showing that pyridazinone rings are only generated in the presence of the ribosome under the conditions used. Third, we demonstrate that the ribosome can also produce oligomers composed of multiple alternating pyridazinone backbones spaced by amide bonds according to a programmed genetic template. Our work represents a starting point for efforts to further elucidate fundamental principles underpinning molecular translation. For example, we observed different levels of incorporation efficiency, which point to future opportunities to engineer the ribosome and associated translation apparatus to work efficiently with the cyclocondensation reaction between γ-keto and α-hydrazino ester monomers. This could teach us how evolution guided ribosome structure and function. While efficiencies of target product range from ˜15-40% for single to multiple pyridazinone bonds, there is room for optimism. Until the advent of Release Factor 1 deficient strains of E. coli less than a decade ago, for example, crude extract based in vitro transcription and translation systems only installed an α-based ncAA ˜20% of the time, with ˜80% truncated product.⁴⁹ Yet, with technological advances these cell-free systems are now closer to 100%.⁵⁰ Looking forward, we expect our work to motivate new directions to expand a broader spectrum of non-canonical linkages in sequence-defined polymers with engineered translation machinery.

### REFERENCES

1. Rodnina, M. V., Beringer, M. & Wintermeyer, W. How ribosomes make peptide bonds. Trends Biochem Sci 32, 20-26 (2007).
2. Wurm, F. M. Production of recombinant protein therapeutics in cultivated mammalian cells. Nature Biotechnology 22, 1393-1398 (2004).
3. Chen, K. & Arnold, F. H. Engineering new catalytic activities in enzymes. Nature Catalysis 3, 203-213 (2020).
4. Corti, D., Purcell, L. A., Snell, G. & Veesler, D. Tackling COVID-19 with neutralizing monoclonal antibodies. Cell 184, 3086-3108 (2021).
5. de la Torre, D. & Chin, J. W. Reprogramming the genetic code. Nature Reviews Genetics 22, 169-184 (2021).
6. Costa, S. A. et al. Active Targeting of Cancer Cells by Nanobody Decorated Polypeptide

Micelle with Bio-orthogonally Conjugated Drug. (2018).
7. Zimmerman, E. S. et al. Production of site-specific antibody-drug conjugates using optimized non-natural amino acids in a cell-free expression system. Bioconjug Chem 25, 351-361 (2014).
8. Kofman, C., Lee, J. & Jewett, M. C. Engineering molecular translation systems. Cell Syst 12, 593-607 (2021).
9. O'Donoghue, P., Ling, J., Wang, Y. S. & Söll, D., Vol. 9 594-598 (Nature Publishing Group, 2013).
10. Chin, J. W. Expanding and reprogramming the genetic code. Nature 550, 53-60 (2017).
11. Arranz-Gibert, P., Vanderschuren, K., Isaacs, F. J., Ledbetter, M. & Romesberg, F. E. Next-generation genetic code expansion. Current Opinion in Chemical Biology 46, xx-yy (2018).
12. Fujino, T., Goto, Y., Suga, H. & Murakami, H. Ribosomal synthesis of peptides with multiple beta-amino acids. J Am Chem Soc 138, 1962-1969 (2016).
13. Katoh, T., Sengoku, T., Hirata, K., Ogata, K. & Suga, H. Ribosomal synthesis and de novo discovery of bioactive foldamer peptides containing cyclic beta-amino acids. Nat Chem (2020).
14. Katoh, T. & Suga, H. Ribosomal elongation of cyclic gamma-amino acids using a reprogrammed genetic code. J Am Chem Soc 142, 4965-4969 (2020).
15. Lee, J., Schwarz, K. J., Kim, D. S., Moore, J. S. & Jewett, M. C. Ribosome-mediated polymerization of long chain carbon and cyclic amino acids into peptides in vitro. Nat Commun 11, 4304 (2020).
16. Lee, J., Tones, R., Byrom, M., Ellington, A. D. & Jewett, M. C. Ribosomal incorporation of cyclic β-amino acids into peptides using in vitro translation. Chem Comm 56, 5597-5600 (2020).
17. Lee, J. et al. Ribosome-mediated incorporation of fluorescent amino acids into peptides in vitro. Chem Commun (Camb) 57, 2661-2664 (2021).
18. Lee, J. et al. Expanding the limits of the second genetic code with ribozymes. Nat Commun 10, 5097 (2019).
19. Ad, O. et al. Translation of Diverse Aramid- and 1,3-Dicarbonyl-peptides by Wild Type Ribosomes in Vitro. Acs Central Science 5, 1289-1294 (2019).
20. Ohta, A., Murakami, H. & Suga, H. Polymerization of alpha-hydroxy acids by ribosomes. Chembiochem 9, 2773-2778 (2008).
21. Tsiamantas, C. et al. Ribosomal Incorporation of Aromatic Oligoamides as Peptide Sidechain Appendages. Angewandte Chemie International Edition 59, 4860-4864 (2020).
22. Maini, R. et al. Ribosomal Formation of Thioamide Bonds in Polypeptide Synthesis. Journal of the American Chemical Society 141, 20004-20008 (2019).
23. Rogers, J. M. & Suga, H. Discovering functional, non-proteinogenic amino acid containing, peptides using genetic code reprogramming. Org Biomol Chem 13, 9353-9363 (2015).
24. Katoh, T. & Suga, H. Consecutive Ribosomal Incorporation of α-Aminoxy/α-Hydrazino Acids with 1/d-Configurations into Nascent Peptide Chains. Journal of the American Chemical Society 143, 18844-18848 (2021).
25. Ohta, A., Murakami, H., Higashimura, E. & Suga, H. Synthesis of polyester by means of genetic code reprogramming. Chem Biol 14, 1315-1322 (2007).
26. Takatsuji, R. et al. Ribosomal Synthesis of Backbone-Cyclic Peptides Compatible with In Vitro Display. Journal of the American Chemical Society 141, 2279-2287 (2019).
27. Englander, M. T. et al. The ribosome can discriminate the chirality of amino acids within its peptidyl-transferase center. Proceedings of the National Academy of Sciences 112, 6038 (2015).
28. Melnikov, S. V. et al. Mechanistic insights into the slow peptide bond formation with D-amino acids in the ribosomal active site. Nucleic Acids Research 47, 2089-2100 (2018).
29. Lim, J. & Anslyn, E. V. submitted.
30. Wafaa, S. H., Hamada, G. E.-G., Kuhnert, N. & Hanafi, H. Z. Chemistry of Pyrazolinones and their Applications. Current Organic Chemistry 16, 373-399 (2012).
31. Song, Y., Zhan, P. & Liu, X. Heterocycle-thioacetic acid motif: a privileged molecular scaffold with potent, broad-ranging pharmacological activities. Curr Pharm Des 19, 7141-7154 (2013).
32. Munoz-Gutierrez, C. et al. Docking and quantitative structure-activity relationship of bi-cyclic heteroaromatic pyridazinone and pyrazolone derivatives as phosphodiesterase 3A (PDE3A) inhibitors. PLoS One 12, e0189213 (2017).
33. Dubey, S. & Bhosle, P. A. Pyridazinone: an important element of pharmacophore possessing broad spectrum of activity. Medicinal Chemistry Research 24, 3579-3598 (2015).
34. Khalil, N. A., Ahmed, E. M., Mohamed, K. O., Nissan, Y. M. & Zaitone, S. A. Synthesis and biological evaluation of new pyrazolone-pyridazine conjugates as anti-inflammatory and analgesic agents. Bioorg Med Chem 22, 2080-2089 (2014).
35. Killian, J. A., Van Cleve, M. D., Shayo, Y. F. & Hecht, S. M. Ribosome-mediated incorporation of hydrazinophenylalanine into modified peptide and protein analogues. Journal of the American Chemical Society 120, 3032-3042 (1998).
36. Morimoto, J., Hayashi, Y., Iwasaki, K. & Suga, H. Flexizymes: their evolutionary history and the origin of catalytic function. Acc Chem Res 44, 1359-1368 (2011).
37. Fleming, S. R. et al. Flexizyme-enabled benchtop biosynthesis of thiopeptides. J Am Chem Soc 141, 758-762 (2019).
38. Katoh, T., Goto, Y., Passioura, T. & Suga, H. in Ribozymes 519-543 (2021).
39. Kang, C. W., Sarnowski, M. P., Elbatrawi, Y. M. & Del Valle, J. R. Access to Enantiopure α-Hydrazino Acids for N-Amino Peptide Synthesis. The Journal of Organic Chemistry 82, 1833-1841 (2017).
40. Armstrong, A., Jones, L. H., Knight, J. D. & Kelsey, R. D. Oxaziridine-Mediated Amination of Primary Amines: Scope and Application to a One-Pot Pyrazole Synthesis. Organic Letters 7, 713-716 (2005).
41. Niwa, N., Yamagishi, Y., Murakami, H. & Suga, H. A flexizyme that selectively charges amino acids activated by a water-friendly leaving group. Bioorg Med Chem Lett 19, 3892-3894 (2009).
42. Shimizu, Y. et al. Cell-free translation reconstituted with purified components. Nature Biotechnology 19, 751-755 (2001).
43. Katoh, T., Iwane, Y. & Suga, H. Logical engineering of D-arm and T-stem of tRNA that enhances d-amino acid incorporation. Nucleic Acids Research 45, 12601-12610 (2017).
44. Katoh, T. & Suga, H. Ribosomal Incorporation of Consecutive β-Amino Acids. Journal of the American Chemical Society 140, 12159-12167 (2018).
45. Katoh, T., Wohlgemuth, I., Nagano, M., Rodnina, M. V. & Suga, H. Essential structural elements in tRNA(Pro) for EF-P-mediated alleviation of translation stalling. Nat Commun 7, 11657 (2016).
46. Peil, L. et al. Lys34 of translation elongation factor EF-P is hydroxylated by YfcM. Nat Chem Biol 8, 695-697 (2012).
47. Dedkova, L. M. et al. beta-Puromycin selection of modified ribosomes for in vitro incorporation of beta-amino acids. Biochemistry 51, 401-415 (2012).
48. Maini, R. et al. Protein Synthesis with Ribosomes Selected for the Incorporation of beta-Amino Acids. Biochemistry 54, 3694-3706 (2015).
49. Hong, S. H. et al. Cell-free protein synthesis from a release factor 1 deficient Escherichia coli activates efficient and multiple site-specific nonstandard amino acid incorporation. ACS Synth Biol 3, 398-409 (2014).
50. Martin, R. W. et al. Cell-free protein synthesis from genomically recoded bacteria enables multisite incorporation of noncanonical amino acids. Nat Commun 9, 1203 (2018)

### Materials and Methods for Example 2

All materials were of the best grade commercially available and used without further purification: 3,5-dinitrobenzyl chloride (Sigma Aldrich, 97%), diisopropylethylamine (DIPEA, Acros, ≥99.5%), chloroacetonitrile (Alfa Aesar, ≥98%), Boc-protected amino acids (Sigma Aldrich, 98%), 3-benzoylpropionic acid (Sigma Aldrich, 99%), 3-benzoylacrylic acid (Sigma Aldrich, 99%), tert-butyl triphenylphosphoranylidenecarbamate (Matrix Scientific, ≥95%), diethyl ketomalonate (Matrix Scientific, ≥95%), Oxone® (Alfa Aesar), trifluoroacetic acid (Alfa Aesar, ≥99.5%). All materials were stored under the recommended storage conditions as described by the supplier. All reaction solvents were purchased from Fischer Scientific, unless otherwise specified. Anhydrous solvents (CH₂Cl₂, DMF, THF, MeOH, and MeCN) were obtained by using the solvent delivery system from Vacuum Atmosphere Company and stored over 3 Å molecular sieves under argon. NMR solvents (CDCl₃, DMSO-d₆, MeOD) were purchased from Cambridge Isotope Laboratories or Sigma-Aldrich. Mass spectra were recorded on a Bruker Rapiflex, Bruker Autoflex, AmaZon SL, or Waters Q-TOF Ultima for electron-spray ionization (ESI) and Impact-II or Waters 70-VSE for electron impact (EI). High resolution mass spectrometry (HRMS) analysis was performed by the University of Texas, Pohang University of Science and Technology (POSTECH), or Korea Advanced Institute of Science and Technology (KAIST) Mass Spectrometry Facility using the 6530 Accurate Mass Q-TOF LC/MS system from Agilent Technologies. ¹H and ¹³C NMR spectra were collected either from Northwestern University, the University of Texas at Austin, POSTECH, or KAIST NMR facility using the Bruker AVANCE III HD 500 MHz cryoprobe NMR spectrometer (NIH grant number: 1 S10 OD021508-01) and processed by TopSpin or MestReNova. Chemical shifts, denoted in ppm, are assigned relative to the residual NMR solvent peaks. Silica gel flash chromatography was performed using 0.035-0.070 mm, 60 Å silica purchased from Acros. Thin layer chromatography was performed using glass silica plates coated with fluorescent indicator (F254) purchased from Merck. Sand, sodium chloride, sodium bicarbonate, potassium carbonate, concentrated hydrochloric acid, and sodium hydroxide pellets were purchased from Fischer Scientific. The 3 Å molecular sieves (4 to 8 mesh, Acros) were activated at 170° C. for at least 24 hours in a vacuum oven and stored in a desiccator.

### Synthetic Procedures

### General Procedure A

Formation of cyanomethyl ester and/or Boc deprotection: To a solution of carboxylic acid (1 equiv.) triethylamine (1.5 equiv.), chloroacetonitrile (1.2 equiv.) and dichloromethane (1.0 M) were added and stirred overnight. After stirring for 16 h at room temperature, the reaction mixture was diluted with EtOAc and washed with HCl (0.5 M aq.), NaHCO₃ (4% (w/v) in water), brine, and dried over MgSO₄. The organic phase was concentrated to provide the crude product. Flash column chromatography was performed when necessary. For deprotection of Boc group, 0.5 mL of TFA dropwise at 0° C. The solution was stirred at room temperature for 1 hour.

### General Procedure B

Formation of dinitrobenzyl esters and/or Boc deprotection: To a solution of carboxylic acid (1 equiv.), dichloromethane (1.0 M), triethylamine (1.5 equiv.), and 3,5-dinotrobenzyl chloride (1.2 equiv.) were added. After stirring for 16 h at room temperature, the reaction mixture was diluted with EtOAc and washed with HCl (0.5 M aq.), NaHCO₃ (4% (w/v) in water), brine, and dried over MgSO₄. The organic phase was concentrated to provide the crude product. The product was purified by flash column chromatography. The resulting fraction containing product was collected in a 100 mL flask and the solvent was removed under reduced pressure. 2 mL of HCl (4N in anhydrous dioxane) was added and let stir for 1 h in room temperature. The resulting product was transferred to a 20 mL glass vial and dried under high vacuum overnight to give final product.

### General Procedure C

Formation of 4-((2-aminoethyl)carbamoyl)benzyl thioates & Boc deprotection: To a solution of carboxylic acid (1.4 equiv.), tert-butyl 2[4-(mercaptomethyl)benzamido]ethyl carbamate (Boc-ABT)² (1.0 equiv), 4-dimethylaminopyridine (DMAP) (2.8 equiv) in DCM was added N-(3-dimethylaminopropyl)-N′-ethylcarbodiimide hydrochloride (EDC·HCl) (2.8 equiv) at 0° C., and the reaction mixture was then warmed to room temperature and stirred for 3 h. To this was added 1 N HCl (aq) and the layers were separated. The aqueous layer was extracted with DCM (×2), and the combined organic layers were dried (MgSO4) and concentrated under reduced pressure. The crude was purified by flash column chromatography (EtOAc/n-Hex) to furnish the Boc-protected products. The deprotection was achieved upon treatment with 4M solution of HCl in 1,4-dioxane, and the resulting products were used without further purification and characterization.

For the following synthesis reactions, ¹H NMR, ¹³C NMR, and HRMS spectral data are not shown.

Synthesis of γ-keto Substrates (1-4)
Cyanomethyl 4-oxo-4-phenylbutanoate (1).

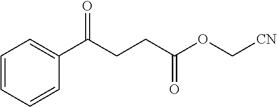 [SMILES: N#CCOC(=O)CCC(=O)c1ccccc1]

Prepared according to the general procedure A using 3-benzoylpropionic acid (200 mg, 1.12 mmol, 1.00 equiv.). A solution of 4-oxo-4-phenylbutanoic acid was dissolved in CH₂Cl₂ (1.12 mL, 1.00 M) and treated with triethylamine (782 μL, 5.61 mmol, 5.00 equiv.) dropwise at 0° C. under an inert atmosphere followed by chloroacetonitrile (214 μL, 3.37 mmol, 3.0 equiv.). Upon complete addition, the reaction was allowed to warm to room temperature and stirred for 18 hours. Upon reaction completion as determined by TLC, the reaction was concentrated in vacuo and the crude material was purified by silica gel flash chromatography (25% EtOAc/Hexanes) to yield the pure product as a clear oil (190 mg, 875 μmol, 77.9% yield).

R_{f}=0.27 (25% EtOAc/Hexanes)

¹H NMR (400 MHz, CDCl₃) δ 7.99-7.94 (m, 2H), 7.61-7.55 (m, 1H), 7.50-7.43 (m, 2H), 4.75 (s, 2H), 3.35 (t, J=6.4 Hz, 2H), 2.84 (t, J=6.6 Hz, 2H).

¹³C NMR (126 MHz, CDCl₃) δ 197.51, 171.55, 136.24, 133.60, 128.81, 128.12, 114.51, 48.59, 33.17, 27.67.

HRMS (ESI/Q-TOF) calc. for C₁₂H₁₁NO₃ [M+Na]⁺=240.0631; Found 240.0631.

Cyanomethyl 4-(4-(methylthio)phenyl)-4-oxobutanoate (2).

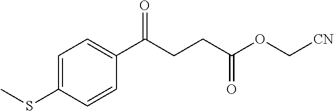 [SMILES: CSc1ccc(C(=O)CCC(=O)OCC#N)cc1]

Prepared according to general procedure A using 4-(4-(methylthio)phenyl)-4-oxobutanoic acid (224 mg, 1 mmol), triethylamine (167 μL, 1.2 mmol), chloroacetonitrile (95 μL, 1.5 mmol) and dichloromethane (0.5 mL). The product was obtained as a yellow oil (205 mg, 78%).

¹H NMR (500 MHz, CD₃OD) 67 7.94 (d, J=8.5 Hz, 2H), 7.35 (d, J=8.5 Hz, 2H), 3.37 (t, J=6.7 Hz, 2H), 2.82 (t, J=6.7 Hz, 2H).

13 C NMR (125 MHz, CD₃OD) 197.5, 171.8, 132.2, 130.0, 128.1 (2C), 124.6 (2C), 114.8, 32.5, 27.0, 14.4, 13.1.

HRMS (ESI/Q-TOF) calc. for C₁₃H₁₃NO₃S [M+Na]⁺=286.0513; Found 286.0511.

3,5-dinitrobenzyl 4-oxopentanoate (3a).

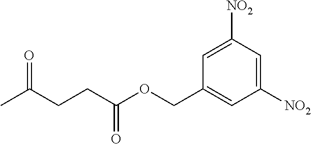 [SMILES: CC(=O)CCC(=O)OCc1cc([N+](=O)[O-])cc([N+](=O)[O-])c1]

Prepared according to general procedure B using 4-oxopentanoic acid (116 mg, 1 mmol), triethylamine (167 μL, 1.2 mmol), 3,5-dinitorbenzyl chloride (324.8 mg, 1.5 mmol) and dichloromethane (0.5 mL). The product was obtained as a white powder (201 mg, 65%).

¹H NMR (500 MHz, CD₃OD) δ 8.96 (s, 1H), 8.65 (d, J=1.7 Hz, 2H), 5.36 (s, 2H), 2.87 (t, 2H), 2.67 (t, 2H), 2.18 (s, 3H).

¹³C NMR (CD₃OD, 125 MHz) δ 208.1, 172.6, 148.5, 140.9, 127.5 (2C), 117.6 (2C), 63.7, 37.2, 28.1, 27.3.

HRMS (ESI/Q-TOF) calc. for C₁₂H₁₂N₂O₇ [M +Na]⁺=319.0542; Found 319.0540.

3,5-dinitrobenzyl 4-oxohexanoate (3b).

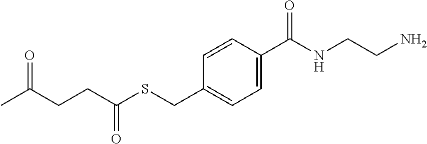 [SMILES: CC(=O)CCC(=O)SCc1ccc(C(=O)NCCN)cc1]

Prepared according to general procedure C using levulinic acid (98 mg, 0.84 mmol), Boc-ABT (186 mg, 0.6 mmol), DMAP (205 mg, 1.7 mmol), EDC·HCl (322 mg, 1.7 mmol) and DCM (4.0 mL). Purification by flash column chromatography (80% EtOAc in n-Hex) afforded the corresponding Boc-protected product as a white solid (146 mg, 60%). The deprotection was achieved upon treatment with 4M solution of HCl in 1,4-dioxane, and the resulting product was used without further purification and characterization. Boc-3b:

¹H NMR (400 MHz, CDCl₃) δ 7.73 (d, J=8.0 Hz, 2H), 7.30 (d, J=8.0 Hz, 2H), 7.25 (br s, 1H), 5.10 (br s, 1H), 4.12 (s, 2H), 3.54-3.50 (m, 2H), 3.39-3.35 (m, 2H), 2.87-2.77 (m, 4H), 2.18 (s, 3H), 1.41 (s, 9H).

¹³C NMR (125 MHz, DMSO-d6) 206.7, 197.6, 166.8, 141.8, 133.2 (2C), 128.9(2C), 128.0 (2C), 39.0 37.9 (2C), 37.5 (2C), 32.2 (2C), 29.9 (3C) ppm.

HRMS (ESI/Q-TOF) calc. for C₂₀H₂₈N₂O₅S [M+K]⁺=447.1356; Found 447.1365.

S-(4-((2-aminoethyl)carbamoyl)benzyl) 4-oxopentanethioate (4).

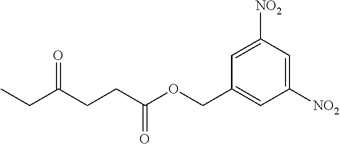 [SMILES: CCC(=O)CCC(=O)OCc1cc([N+](=O)[O-])cc([N+](=O)[O-])c1]

Prepared according to general procedure B using 4-oxo-4-phenylbutanoic acid (178 mg, 1 mmol), triethylamine (167 μL, 1.2 mmol), chloroacetonitrile (95 μL, 1.5 mmol) and dichloromethane (0.5 mL). The product was obtained as a white powder (xx mg, 51%).

¹H NMR (500 MHz, MeOD) δ 8.96 (s, 2H), 8.65 (s, 2H), 5.36 (s, 2H), 2.83 (t, J=6.3 Hz, 2H), 2.68 (t, J=6.0 Hz, 2H), 2.83 (t, J=Hz, 2H), 2.52 (q, J=7.3 Hz, 2H), 1.03 (t, J=7.3 Hz, 3H).

¹³C NMR (125 MHz, MeOD) 210.5, 172.6, 148.6 (2C), 140.9, 127.5 (2C), 117.6, 63.7, 36.0, 34.9, 27.3, 6.5 (2C) ppm.

HRMS (ESI/Q-TOF) calc. for C₁₃H₁₄N₂O₇ [M+Na]⁺=333.0699; Found 333.0684.

### Synthesis of Oxaziridine is Shown in FIG. 19.

2-(tert-butyl) 3,3-diethyl 1,2-oxaziridine-2,3,3-tricarboxylate (Boc-Ozd)^{1, 3}

Oxaziridine was synthesized using previously reported methods¹. A pressure flask containing N-Boc-iminophosphorane (9.76 g, 25.9 mmol, 1.00 equiv.) in 26 mL of anhydrous THF was treated with diethyl ketomalonate (3.94 mL, 25.9 mmol, 1.00 equiv.). The reaction mixture was sealed and stirred at 60° C. After 24 hours, the mixture was cooled and concentrated in vacuo. The light-yellow oil was redissolved in warm toluene and Ph₃PO was precipitated with pentane. The supernatant was filtered, and the filtrate was concentrated in vacuo. This process was repeated 3-4 times or until no more Ph₃PO precipitate was observed. Concentration in vacuo gave the N-Boc-iminodiethylmalonate as a light-yellow oil (5.35 g, 19.6 mmol, 76% yield. Without further purification, the N-Boc-iminodiethylmalonate was dissolved in 74 mL of MeCN and 48 mL of H₂O before addition of a solid mixture of Oxone (28.9 g, 47.0 mmol, 2.40 equiv.) and NaHCO₃ (6.09 g, 72.5 mmol, 3.70 equiv.). The reaction mixture was stirred for 5 hours before addition of another portion of Oxone (28.9 g, 47.0 mmol, 2.40 equiv.). The reaction was stirred for an additional 19 hours under ambient conditions. The heterogeneous mixture was diluted with 300 mL of H₂O and extracted 3 times with CH₂Cl₂. The combined organic layers were dried over MgSO₄, filtered, and concentrated in vacuo. Purification by silica gel flash chromatography (6:2.5:0.5 Hexane/CH₂Cl₂/Et₂O) to yield a light-yellow oil (1.84 g, 6.37 mmol, 32.5% yield). Spectra matched literature.

### Synthesis of HzPhe-CME HCl (5) is Shown in FIG. 20.

((tert-butoxycarbonyl)amino)-L-phenylalanine.¹

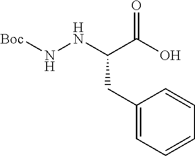 [SMILES: CC(C)(C)OC(=O)NN[C@@H](Cc1ccccc1)C(=O)O]

To a biphasic mixture of L-phenylalanine (357 mg, 2.16 mmol, 1.00 equiv.) in TRF (20 mL) and satd. NaHCO₃ (aq) (20 mL) was added Boc Ozd (625 mg, 2.16 mmol, 1.00 equiv.) dropwise. The reaction was allowed to stir for 4 hours under ambient conditions before treatment with ethylenediamine (550 μL, 8.21 mmol, 3.8 equiv.). After 5 minutes, the reaction mixture was acidified to pH ˜1 using 1M HCl (aq), extracted with EtOAc, and concentrated in vacuo to yield a white solid that quickly turned light brown. Trituration with EtOAc and hexanes gave the desired product as a white solid (424 mg, 1.51 mmol, 70.0% yield). Spectra matched literature.

¹H NMR (400 MHz, DMSO-d₆) δ 8.28 (s, 1H), 7.30-7.15 (m, 5H), 3.68 (t, J=6.3 Hz, 1H), 2.84 (d, J=6.3 Hz, 2H), 1.37 (s, 9H).

tert-butyl (S)-2-(1-(cyanomethoxy)-1-oxo-3-phenylpropan-2-yl)hydrazine-1-carboxylate.

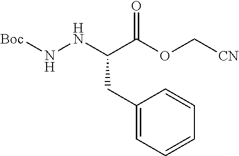 [SMILES: CC(C)(C)OC(=O)NN[C@@H](Cc1ccccc1)C(=O)OCC#N]

A solution of Boc-HzPhe-OH (250 mg, 892 μmol, 1.00 equiv.) in 1.5 mL of anhydrous DMF was treated with diisopropylethylamine (171 μL, 981 μmol, 1.10 equiv.) then chloroacetonitrile (62.2 μL, 981 μmol, 1.10 equiv.). The reaction mixture was stirred under an inert atmosphere at room temperature for 18 hours then concentrated in vacuo. The crude material was redissolved in EtOAc and purified by silica gel flash chromatography (30% EtOAc/Hexanes) to yield a clear oil (270 mg, 845 μmol, 94.8%).

R_{f}=0.30 (30% EtOAc/Hexanes).

1H NMR (500 MHz, CDCl₃) δ 7.37-7.21 (m, 5H), 6.31 (s, 1H), 4.71 (d, J=2.8 Hz, 2H), 4.08 (s, 1H), 4.05 (t, J=7.0 Hz, 1H), 3.06 (qd, J=14.0, 7.0 Hz, 2H), 1.45 (s, 9H).

¹³C NMR (126 MHz, CDCl₃) δ 171.20, 156.54, 135.77, 129.21, 128.87, 127.33, 114.10, 81.25, 64.29, 48.75, 36.86, 28.33.

HRMS (ESI/Q-TOF) calc. for C₁₆H₂₁N₃O₄ [M+Na]⁺=342.1424; Found 342.1435.

Cyanomethyl amino-L-phenylalaninate HCl. (5)

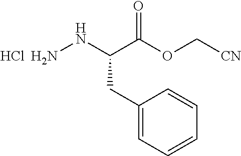 [SMILES: Cl ; N#CCOC(=O)[C@H](Cc1ccccc1)NN]

Prepared according to general procedure A using Boc-HzPhe-CME. To a vial containing Boc-hydPhe-CME (100 mg, 313 μmol, 1.00 equiv.) in 2 mL of MeCN was added dropwise 4 M HCl in dioxane (235 μL, 939 μmol, 3.00 equiv.) at 0° C. under argon. The solution was stirred at 0° C. for 2 hours and concentrated in vacuo. The residue was dissolved in a minimal amount of MeCN and the product was precipitated by addition of Et₂O. The solids were washed 3 times with 15% MeOH in Et₂O and dried in vacuo to yield the hydrochloride salt as a white solid which was used without further purification.

HRMS (ESI/Q-TOF) calc. for C₁₁H₁₃N₃O₂ [M+H]⁺=220.1081; Found 220.1081.

3,5-dinitrobenzyl (tert-butoxycarbonyl)-L-alaninate (Boc-Ala-DNB).

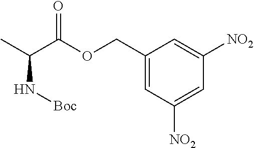 [SMILES: C[C@H](NC(=O)OC(C)(C)C)C(=O)OCc1cc([N+](=O)[O-])cc([N+](=O)[O-])c1]

Prepared using General Method B. Purification by silica gel flash chromatography (20% EtOAc/Hexanes) gave the title compound as a yellow tinted white solid (0.908 g, 82% yield).

R_{f}=0.21 (20% EtOAc/Hexane)

¹H NMR (500 MHz, CDCl₃) δ 9.01 (t, J=2.1 Hz, 1H), 8.56 (d, J=2.0 Hz, 2H), 5.41-5.31 (m, 2H), 4.96 (br s, 1H), 4.44-4.34 (m, 1H), 1.44 (d, J=7.3 Hz, 3H), 1.43 (s, 9H).

¹³C NMR (126 MHz, CDCl₃) δ 173.16, 155.29, 148.85, 140.24, 127.91, 118.75, 80.49, 64.52, 49.44, 28.39, 18.26.

HRMS (ESI/Q-TOF) calc. for C₁₅H₁₉N₃O₈ [M+Na]⁺=392.1064; Found 392.1065.

3,5-dinitrobenzyl L-alaninate TFA (Ala-DNB).

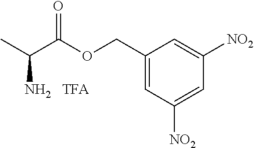 [SMILES: C[C@H](N)C(=O)OCc1cc([N+](=O)[O-])cc([N+](=O)[O-])c1]

To a vial containing Boc-Ala-DBE (500 mg, 1.35 mmol, 1.0 equiv.) dissolved in CH₂Cl₂ (4.0 mL) was added TFA (1.0 mL, 13.5 mmol, 10.0 equiv.) dropwise at 0° C. After complete addition, the reaction was warmed to room temperature and stirred for 30 min upon which TLC analysis confirmed reaction completion. The volatiles were removed in vacuo and the residue was triturated with Et₂O to yield the pure product as a white solid. (483 mg, 93% yield).

1H NMR (500 MHz, DMSO) δ 8.82 (t, J=2.1 Hz, 1H), 8.73 (d, J=2.1 Hz, 2H), 8.58 (s, 3H), 5.50 (d, J=1.9 Hz, 2H), 4.26 (q, J=7.2 Hz, 1H), 1.46 (d, J=7.2 Hz, 3H).

13C NMR (126 MHz, DMSO-d6) δ 169.70, 148.14, 139.74, 128.45, 118.41, 64.97, 47.97, 15.71.

HRMS (ESI/Q-TOF) calc. for C₁₀H₁₁N₃O₆ [M+H]⁺=270.0721; Found 270.0727.

tert-butyl (S)-2-(1-((3,5-dinitrobenzyl)oxy)-1-oxopropan-2-yl)hydrazine-1-carboxylate (6a).

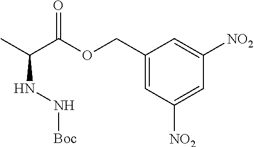 [SMILES: C[C@H](NNC(=O)OC(C)(C)C)C(=O)OCc1cc([N+](=O)[O-])cc([N+](=O)[O-])c1]

To a biphasic mixture of L-alanine-DBE TFA (132 mg, 346 μmol, 1.00 equiv.) in THF (2.5 mL) and satd. NaHCO₃ (aq) (2.5 mL) was added oxaziridine (100 mg, 346 μmol, 1.00 equiv.) dropwise. The reaction was allowed to stir for 120 min under ambient conditions before the reaction mixture was extracted three times with 20 mL of EtOAc. The combined organic layers were dried over anhydrous Na₂SO₄ and concentrated in vacuo to yield a clear oil. The product was purified by silica gel flash chromatography (35% EtOAc/Hexanes) to yield the product as a yellow-tinted oil (114 mg, 297 μmol, 85.8% yield).

R_{f}=0.24 (35% EtOAc/Hexanes).

¹H NMR (500 MHz, CDCl₃) δ 8.96-8.93 (m, 1H), 8.55-8.51 (m, 2H), 6.40 (s, 1H), 5.34 (s, 2H), 4.90 (s, 1H), 4.41-4.15 (m, 1H), 1.51-1.28 (m, 12H).

¹³C NMR (126 MHz, CDCl₃) δ 173.21, 156.67, 148.65, 140.25, 127.92, 118.58, 81.03, 64.27, 58.44, 28.23, 15.91.

HRMS (ESI/Q-TOF) calc. for C₁₅H₂₀N₄O₈ [M+Na]⁺=407.1173; Found 407.1180.

3,5-dinitrobenzyl amino-L-alaninate HCl (6a).

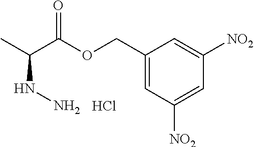 [SMILES: C[C@H](NN)C(=O)OCc1cc([N+](=O)[O-])cc([N+](=O)[O-])c1 ; Cl]

A dram vial equipped with a stirring rod was charged with Boc-hydAla-DBE (20.0 mg, 52.0 μmol, 1.00 equiv.) dissolved in 500 μL of anhydrous CH₂Cl₂. The vial was placed in an ice bath and cooled to 0° C. before it was treated with 50 μL of TFA (649 μmol, 12.5 equiv.). After removing the reaction from the ice bath and allowing it to slowly warm to 23° C., the solution was stirred for 120 minutes then concentrated in vacuo. The residue was redissolved in ˜200 μL of Et₂O and treated with 200 μL of 2N HCl in Et₂O to produce a cloudy white heterogeneous mixture. The resulting solids were allowed to settle, and the supernatant was carefully removed via pipette. The precipitate was washed 3 more times with ˜1.0 mL of Et₂O before being dried in vacuo to yield the HCl product as a light-yellow solid. The compound was used without further purification and characterization. (13.3 mg, 79.7% yield).

HRMS (ESI/Q-TOF) calc. for C₁₀H₁₂N₄O₆ [M+H]⁺=285.0830; Found 285.0833.

### Synthesis of HzAla-ABT (6)

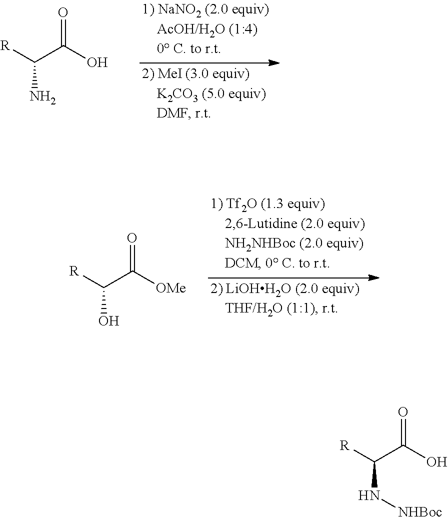

[(tert-Butoxycarbonyl)amino]-L-alanine

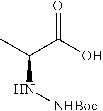

Synthesized according to a previously reported procedure¹; (+)-Methyl D-lactate was obtained from a commercial supplier (Sigma-Aldrich) and used as received: To a solution of (+)-methyl D-lactate (1.43 mL, 15.0 mmol, 1.0 equiv) in DCM (45 mL) was added trifluoromethanesulfonic anhydride (3.28 mL, 19.5 mmol, 1.3 equiv) and 2,6-lutidine (3.47 mL, 30.0 mmol, 2.0 equiv) at 0° C., and the reaction was stirred at the same temperature until full consumption of the starting material (confirmed by TLC). To this was then added tert-butyl carbazate (3.96 g, 30.0 mmol, 2.0 equiv), and the resulting mixture was further stirred at 0° C. for 4 h, then at room temperature for 16 h. The reaction mixture was diluted with DCM and washed with H₂O, brine and 1 M HCl_{(aq)}. The organic layer was then dried over anhydrous MgSO₄, concentrated under reduced pressure, and purified by flash column chromatography (30% EtOAc/n-Hex) to furnish [(tert-butoxycarbonyl)amino]-L-alanine methyl ester as a pale yellow oil (2.64 g, 81%).

The methyl ester (2.44 g, 11.2 mmol, 1.0 equiv) obtained above was then dissolved in 1:1 mixture of THF/H₂O (24 mL) and treated with LiOH·H₂O (940 mg, 22.4 mmol, 2.0 equiv). After stirring at room temperature for 3 h, the mixture was concentrated under reduced pressure and the remaining aqueous layer was washed with Et₂O. The aqueous layer was then acidified to pH ˜1 using 1M HCl_{(aq)}, extracted with EtOAc, dried over anhydrous MgSO₄, and concentrated under reduced pressure to give [(tert-butoxycarbonyl)amino]-L-alanine as a thick colorless oil (2.08 g, 91%). Data consistent with those previously reported.

S-(4-((2-aminoethyl)carbamoyl)benzyl) (R)-2-hydrazineylpropanethioate (6b).

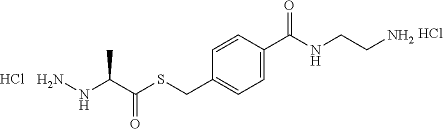 [SMILES: Cl ; C[C@H](NN)C(=O)SCc1ccc(C(=O)NCCN)cc1 ; Cl]

Prepared according to General Procedure C using [(tert-butoxycarbonyl)amino]-L-alanine¹ (428 mg, 2.1 mmol), Boc-ABT (465 mg, 1.5 mmol), DMAP (512 mg, 4.2 mmol), EDC·HCl (803 mg, 4.2 mmol) and DCM (10 mL). Purification by flash column chromatography (60% EtOAc in n-Hex) afforded the corresponding Boc-protected product as a colorless oil (338 mg, 45%). The deprotection was achieved upon treatment with 4M solution of HCl in 1,4-dioxane, and the resulting product was used without further purification and characterization. Boc-6b:

¹H NMR (400 MHz, CDCl₃) δ 7.74 (d, J=8.0 Hz, 2H), 7.34 (d, J=8.0 Hz, 2H), 7.20 (br s, 1H), 6.26 (br s, 1H), 5.02 (br s, 1H), 4.09 (s, 2H), 3.79 (q, J=7.0 Hz, 1H), 3.56-3.52 (m, 2H), 3.41-3.37 (m, 2H), 2.28 (br s, 1H), 1.44 (s, 9H), 1.42 (s, 9H), 1.31 (d, J=7.0 Hz, 3H).

¹³C NMR (101 MHz, CDCl₃) δ 203.1, 167.5, 157.7, 156.8, 141.4, 133.2, 129.1, 127.5, 81.3, 80.2, 66.2, 42.2, 40.1, 32.5, 28.5, 28.4, 17.3.

HRMS [Method] Calculated for C₂₃H₃₆N₄O₆S [M+H]⁺: 497.2428, Found: 497.2434.

### Synthesis of AOP (7) is Shown in FIG. 21.

Cyanomethyl 2-((tert-butoxycarbonyl)amino)-4-oxo-4-phenylbutanoate (Racemic).

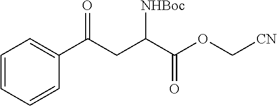 [SMILES: CC(C)(C)OC(=O)NC(CC(=O)c1ccccc1)C(=O)OCC#N]

Prepared according to General Procedure A. A solution of 2-((tert-butoxycarbonyl)amino)-4-oxo-4-phenylbutanoic acid (50 mg, 170 μmol, 1.00 equiv.) dissolved in 500 μL of anhydrous DMF was treated with DIPEA (148 μL, 852 μmol, 5.00 equiv.) then chloroacetonitrile (33 μL, 511 μmol, 3.00 equiv.) at 0° C. The reaction mixture was allowed to warm to room temperature and stirred for 18 hours. The mixture was concentrated in vacuo and the crude material was purified by silica gel flash chromatography (30% EtOAc/Hexanes) to afford the corresponding product as a colorless oil (53.3 g, 160 μmol, 94.1% yield).

R_{f}=0.25 (30% EtOAc/Hexane)

¹H NMR (500 MHz, CDCl₃) δ 7.93 (d, J=7.2 Hz, 2H), 7.60 (t, J=7.4 Hz, 1H), 7.48 (t, J=7.7 Hz, 2H), 5.59 (d, J=9.0 Hz, 1H), 4.85-4.70 (m, 3H), 3.74 (dd, J=18.3, 4.4 Hz, 1H), 3.58 (dd, J=18.3, 4.0 Hz, 1H), 1.43 (s, 9H).

¹³C NMR (126 MHz, CDCl₃) δ 197.57, 170.58, 155.50, 135.75, 134.13, 128.92, 128.31, 114.03, 80.57, 49.40, 49.35, 41.15, 28.35.

HRMS (ESI/Q-TOF) calc. for C₁₇H₂₀N₂O₅ [M+Na]⁺=355.1264; Found 355.1275.

Cyanomethyl 2-amino-4-oxo-4-phenylbutanoate TFA salt (Racemic). (7)

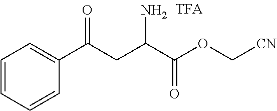 [SMILES: N#CCOC(=O)C(N)CC(=O)c1ccccc1]

The Boc-protected amino ester (25.0 mg, 72.0 μmol) was dissolved in 5 mL of CH₂Cl₂ and treated with 0.5 mL of TFA dropwise at 0° C. The solution was stirred at room temperature for 1 hour after which the volatiles were removed in vacuo. The off-white waxy residue was triturated with Et₂O to afford a white solid powder after filtration. (12.2 mg, 49.3 μmol, 68.5%).

¹H NMR (400 MHz, DMSO-d₆) δ 8.50 (s, 3H), 8.02-7.97 (m, 2H), 7.77-7.68 (m, 1H), 7.64-7.55 (m, 2H), 5.14 (s, 2H), 4.67 (t, J=4.7 Hz, 1H), 3.80 (d, J=5.0 Hz, 2H).

¹³C NMR (126 MHz, DMSO-d₆) δ 195.75, 168.45, 135.09, 134.26, 128.98, 128.21, 115.25, 50.30, 47.64, 38.58.

HRMS (ESI/Q-TOF) calc. for C₁₂H₁₂N₂O₃ [M+Na]⁺=255.0740; Found 255.0742.

### Preparation of Materials for Cell-Free Protein Translation

### Preparation for DNA Templates for RNAs

The DNA templates for flexizyme and tRNAs preparation were synthesized by using the following primers as previously described.⁴

Sequence of the final DNA templates used for in vitro transcription by the T7 RNA polymerase:

| | |
|---|---|
| fMet | GTAATACGACTCACTATAGGCGGGGTGGAGCAGCCTGGTAGCTCGTCGGGCTCATAAC |
| (CAU) | CCGAAGATCGTCGGTTCAAATCCGGCCCCCGCAACCA (SEQ ID NO: 15) |
| Pro1E2 | GTAATACGACTCACTATAGGGTGATTGGCGCAGCCTGGTAGCGCACTTCGTTGGTAAC |
| (GGU) | GAAGGGGTCAGGGGTTCGAATCCCCTATCACCCGCCA (SEQ ID NO: 16) |

*Note that the underlined sequences are the T7 promoter sequence.

Preparation of Fx and tRNAs

Flexizymes and tRNAs were prepared using the HiScribe™ T7 High yield RNA synthesis kit (NEB, E2040S) and purified by the previously reported methods⁴.

### General Fx-Medicated Acylation Reaction

### 1) Microhelix

1 μL of 0.5 M HEPES (pH 7.5) or bicine (pH 8.8), 1 μL of 10 μM microhelix, and 3 μL of nuclease-free water were mixed in a PCR tube with 1 μL of 10 μM eFx, dFx, and aFx, respectively. The mixture was heated for 2 min at 95° C. and cooled down to room temperature over 5 min. 2 μL of 300 mM MgCl₂ was added to the cooled mixture and incubated for 5 min at room temperature. Followed by the incubation of the reaction mixture on ice for 2 min, 2 μL of 25 mM activated ester substrate in DMSO was then added to the reaction mixture. The reaction mixture was further incubated for 6-48 h on ice in cold room.

2) tRNA

2 μL of buffer (0.5 M HEPES (pH 7.5) or 0.5 M bicine), 2 μL of 250 μM tRNA, 2 μL of 250 μM of a Fx selected on the microhelix experiment, and 6 μL of nuclease-free water were mixed in a PCR tube. The mixture was heated for 2 min at 95° C. and cooled down to room temperature over 5 min. 4 μL of 300 mM MgCl₂ was added to the cooled mixture and incubated for 5 min at room temperature. Followed by the incubation of the reaction mixture on ice for 2 min, 4 μL of 25 mM activated ester substrate in DMSO was then added to the reaction mixture. The reaction mixture was further incubated for the optimal time determined on the microhelix experiment on ice in cold room.

### In Vitro Synthesis of Pyridazinone

### 1) N-Terminal Incorporation

As a reporter peptide, a T7 promoter-controlled DNA template (pJL1_MT_StrepII) was designed to encode a streptavidin (Strep) tag and additional Met (AUG-X) and Thr (ACC-Y) codons (XYWHSPQFEK (SEQ ID NO: 17)). The initiation codon AUG and ACC were used for N-terminal incorporation of the γ-keto and hydrazineyl ester substrates, respectively). The PURExpress™ Δ (aa, tRNA) kit (NEB, E6840S) was used for pyridazinone formation reaction and the reaction was performed with only the 8 amino acids that decode the purification tag. The reaction mixtures were incubated at 37° C. for 2 h. The synthesized peptides were then purified using Strep-Tactin®-coated magnetic beads (IBA) and characterized by MALDI-TOF mass spectroscopy.

### 2) C-Terminal Incorporation (Alternating Consecutive Incorporation)

For alternating incorporations at the C-terminal region of a peptide, the pJL1-StrepII_TI2 and pJL1-StrepII_TI3 encoding the same amino acids (MWHSPQFEKSXYXY (SEQ ID NO: 18) or MWHSPQFEKSXYXYXY (SEQ ID NO: 19)), where X (Thr:ACC) and Y (Ile:AUC) indicate the position of the γ-keto amino acid (7) and (S)-HzAla (6) substrates, respectively. The reaction condition, purification and characterization methods are the same with the methods described in the paragraph above.

### 3) Effect of Other Translational Machinery for Pyridazinone Bond Formation

For this study, a custom-made PURExpress® Δ (aa, tRNA, ribosome) kit (NEB, E3315Z) and the wildtype ribosome provided in the kit was not used. To investigate the engineered ribosome's effect, 15 μM (final concentration) of the engineered ribosome (Hecht's 040329)⁵ was added to the reaction mixture that contains the 8 amino acids decoding the strep-tag. To investigate the EF-P's effect, additional 10 μM of EF-P 6 was added into the reaction mixture. The reaction condition, purification, and characterization methods are the same with the methods described in the paragraph above.

LC-MS analysis of pyridazinone. After 2 h at 37° C., NaOH (5 mM in final) was added to cleave the tRNA ester linkage of 1 and 5, or the resulting pyridazinone (2-(6-oxo-3-phenyl-5,6-dihydropyridazin-1(4H)-yl)-3-phenylpropanoic acid, OPDP) from the tRNA.

### REFERENCES FOR MATERIALS AND METHODS

1. Kang, C. W., Sarnowski, M. P., Elbatrawi, Y. M. & Del Valle, J. R. Access to Enantiopure alpha-Hydrazino Acids for N-Amino Peptide Synthesis. J Org Chem 82, 1833-1841 (2017).
2. Niwa, N., Yamagishi, Y., Murakami, H. & Suga, H. A flexizyme that selectively charges amino acids activated by a water-friendly leaving group. Bioorg Med Chem Lett 19, 3892-3894 (2009).
3. Armstrong, A., Jones, L. H., Knight, J. D. & Kelsey, R. D. Oxaziridine-mediated amination of primary amines: scope and application to a one-pot pyrazole synthesis. Org Lett 7, 713-716 (2005).
4. Lee, J. et al. Expanding the limits of the second genetic code with ribozymes. Nat Commun 10, 5097 (2019).
5. Maini, R. et al. Protein Synthesis with Ribosomes Selected for the Incorporation of beta-Amino Acids. Biochemistry 54, 3694-3706 (2015).
6. Katoh, T., Wohlgemuth, I., Nagano, M., Rodnina, M. V. & Suga, H. Essential structural elements in tRNA(Pro) for EF-P-mediated alleviation of translation stalling. Nat Commun 7, 11657 (2016).

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. An acylated tRNA molecule having a Formula I(a) or II(a): 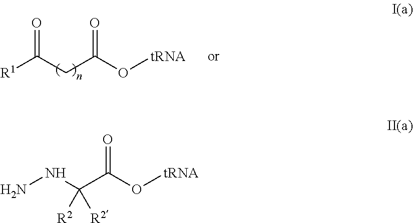
wherein:
n is 1-10, 1-9, 1-8, 1-7, 1-6, 1-5, 1-4, or 1-3;
R¹ is hydrogen, alkyl (e.g., methyl, ethyl), aryl (e.g., phenyl) which optionally is substituted at one or more positions with alkyl or alkylthio (e.g., 4-methylthio-phenyl),
R² is hydrogen, alkyl (e.g., methyl, isopropyl), alkylaryl (e.g., benzyl) which optionally is substituted at one or more positions with hydroxyl (e.g., 3,4-dihydroxy-benzyl), or R2 is the side chain of an amino acid (e.g., a side chain of an amino acid selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine; and
R^{2′} is hydrogen or alkyl (e.g., methyl).

2. The acylated tRNA molecule of claim 1, having a formula selected from:

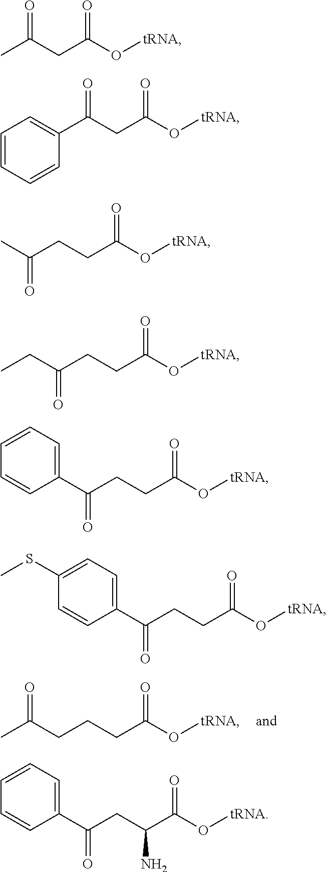

3. The acylated tRNA molecule of claim 1, having a formula selected from:

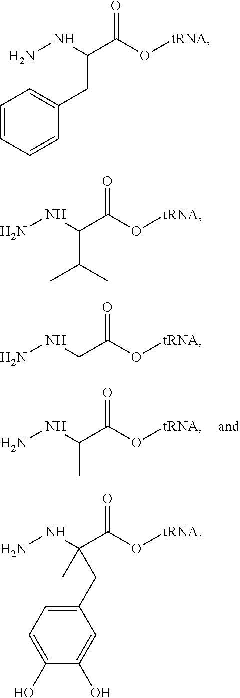

4. The acylated tRNA molecule of claim 1, having a formula selected from:

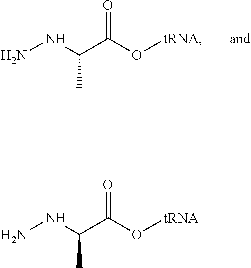

5. The acylated tRNA molecule of claim 1, having a formula selected from:

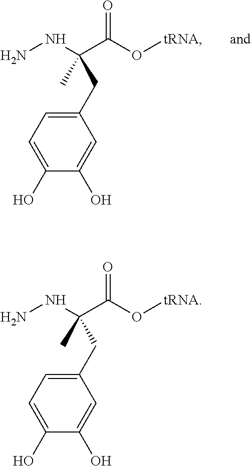

6. A compound or molecule having a Formula III: 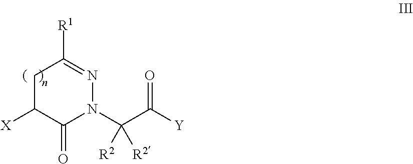
wherein:
X is hydrogen or the C-terminus of a polymer chain (e.g., the C-terminus of a polypeptide chain);
n is 0-8, 0-7, 0-6, 0-5, 0-4, 0-3, or 0-2;
R¹ is hydrogen, alkyl (e.g., methyl, ethyl), aryl (e.g., phenyl) which optionally is substituted at one or more positions with alkyl or alkylthio (e.g., 4-methylthio-phenyl), or R¹ is the C-terminus of a peptide chain;
R² is hydrogen, alkyl (e.g., methyl, isopropyl), alkylaryl (e.g., benzyl) which optionally is substituted at one or more positions with hydroxyl (e.g., 3,4-dihydroxy-benzyl), or R² is the side chain of an amino acid (e.g., a side chain of an amino acid selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine;
R^{2′} is hydrogen or alkyl (e.g., methyl);
Y is hydrogen or the N-terminus of a polymer chain (e.g., the N-terminus of a polypeptide chain) or Y has a formula selected from —O(tRNA), —O(R³), or —NH(R³), wherein R³ is selected from hydrogen and alkyl.

7. The compound of claim 6, having a formula selected from:

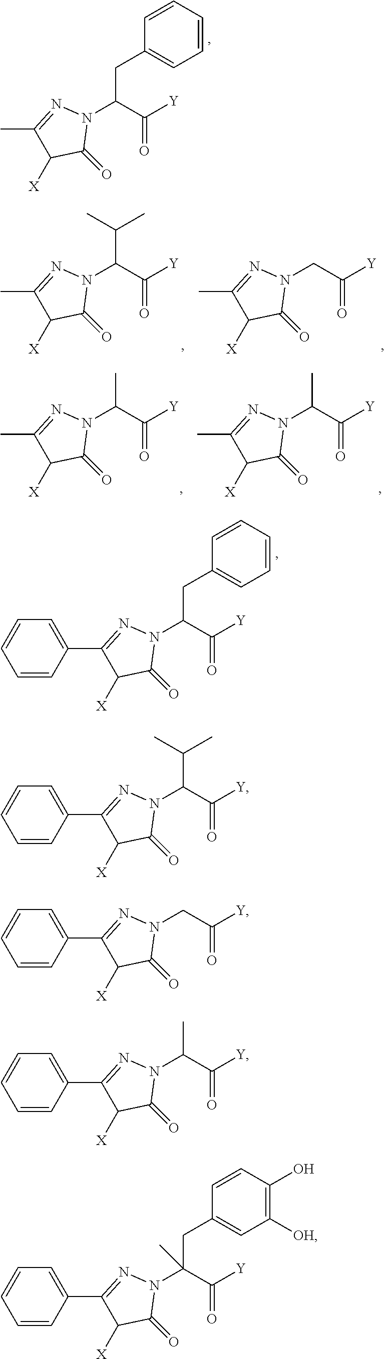

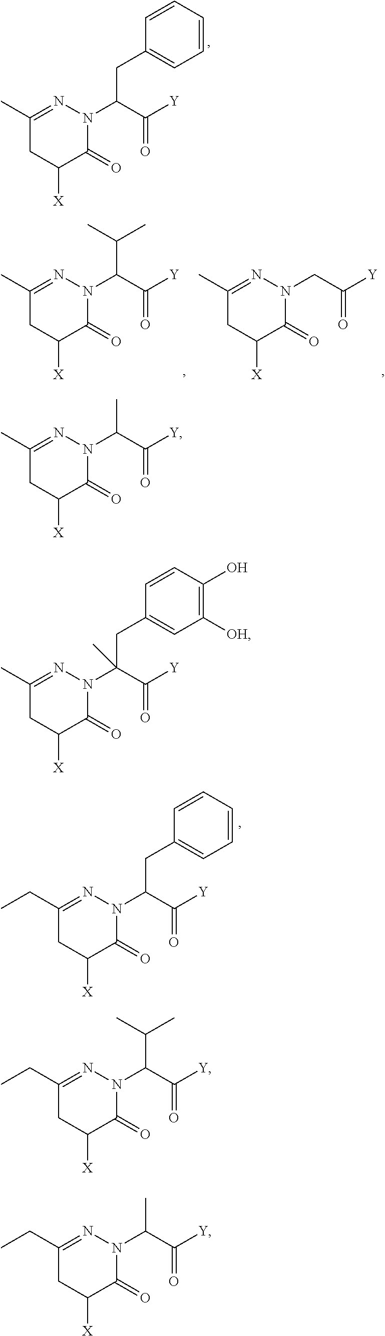

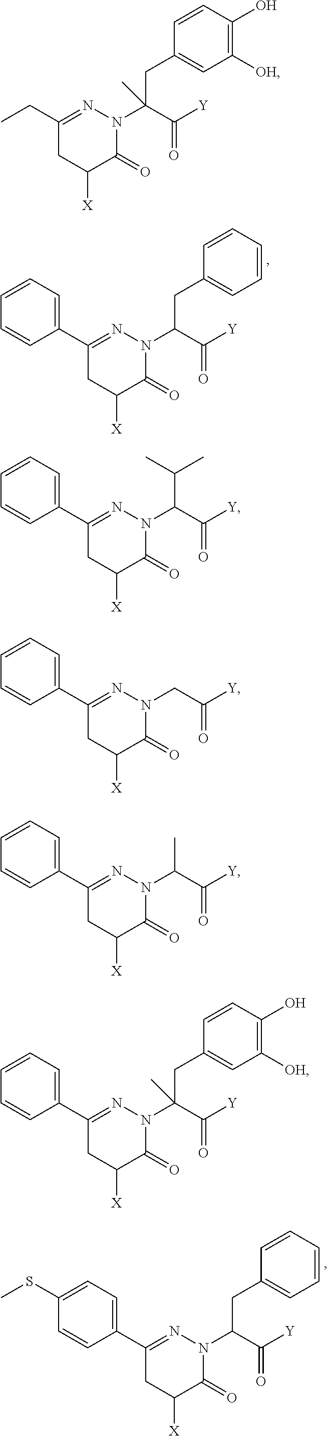

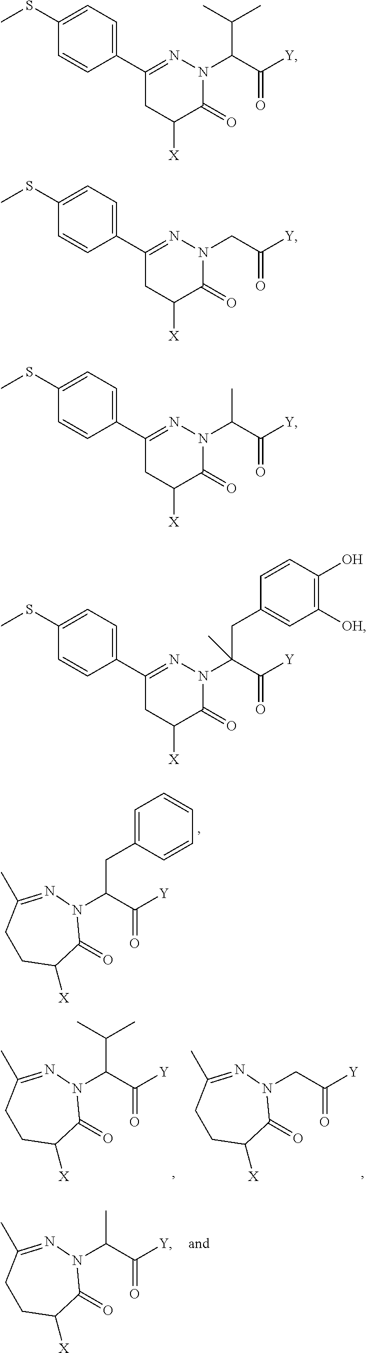

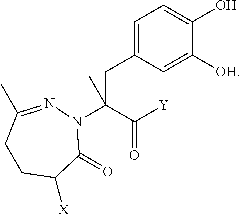

8. The compound or molecule of claim 6 having a formula selected from:

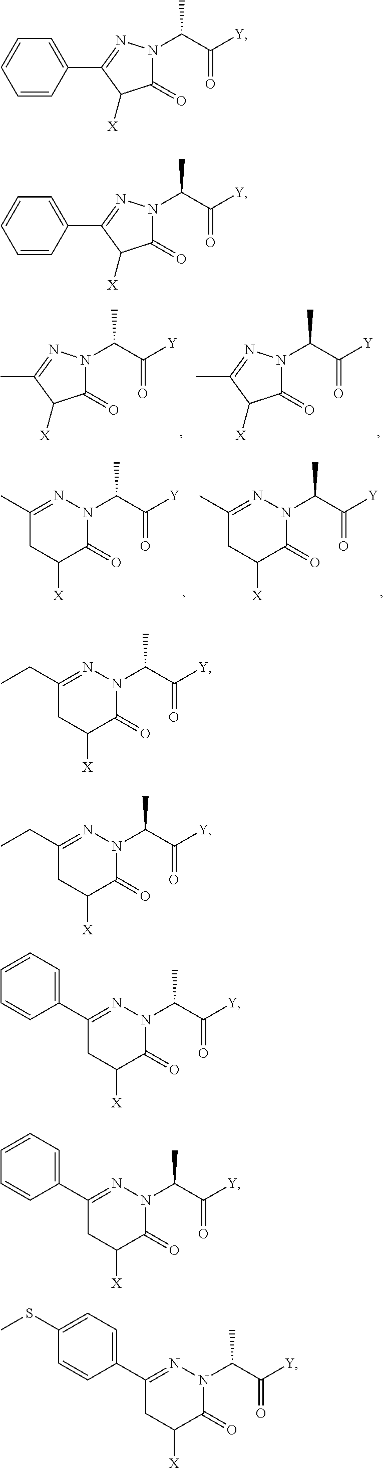

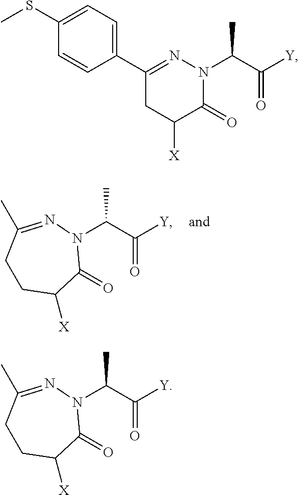

9. The compound or molecule of claim 6 having a formula selected from:

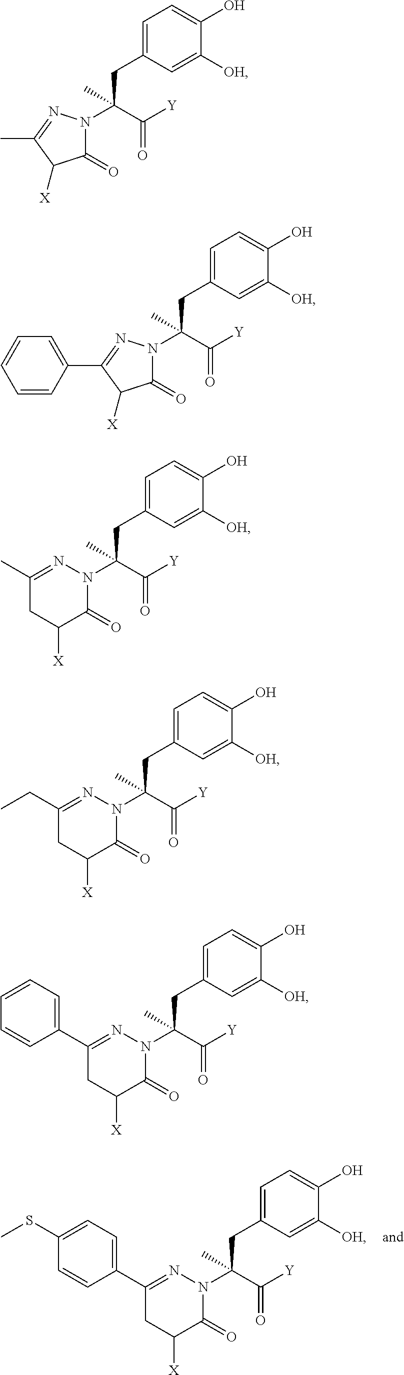

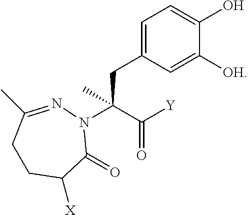

10. A method for preparing a sequence defined polymer via translating an mRNA, wherein the mRNA comprises a codon corresponding to an anticodon of an acylated tRNA molecule having a Formula I(a) or Formula II(a): 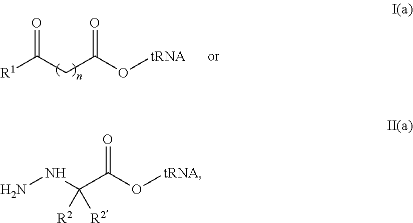
wherein:
n is 1-10, 1-9, 1-8, 1-7, 1-6, 1-5, 1-4, or 1-3; and
R¹ is hydrogen, alkyl (e.g., methyl, ethyl), aryl (e.g., phenyl) which optionally is substituted at one or more positions with alkyl or alkylthio (e.g., 4-methylthio-phenyl), and wherein the method comprises incorporating the chemical moiety of the acylated tRNA having a Formula I(a) into the polymer via translation;
R² is hydrogen, alkyl (e.g., methyl, isopropyl), alkylaryl (e.g., benzyl) which optionally is substituted at one or more positions with hydroxyl (e.g., 3,4-dihydroxy-benzyl), or R2 is the side chain of an amino acid (e.g., a side chain of an amino acid selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine;
R^{2′} is hydrogen or alkyl (e.g., methyl).

11. The method of claim 10, wherein the mRNA comprises a codon corresponding to an anticodon of an acylated tRNA molecule having the Formula I(a) and the mRNA further comprises a codon corresponding to an anticodon of an acylated tRNA molecule having the Formula II(a); and wherein in the mRNA the codon for the acylated tRNA having Formula II(a) is located immediately 3′ to the codon for the acylated tRNA having Formula I(a) and the method further comprising incorporating the chemical moiety of the acylated tRNA having Formula II(a) into the polymer via translation and conjugation with the chemical moiety of the acylated tRNA having Formula I(a).

12. The method of claim 11, wherein the chemical moiety of the acylated tRNA having Formula II(a) is conjugated to the chemical moiety of the acylated tRNA having Formula I(a) to form a linkage comprising an optionally substituted pyrazolone group, optionally substituted pyridazinone group, or an optionally substituted diazepinone group.

13. The method of claim 11, wherein the method is performed in vitro.

14. The method of claim 11, wherein the method is performed in vivo.

15. The method of claim 11, wherein the codon for the acylated tRNA having Formula I(a) is a codon for an N-terminal methionine.

16. The method of claim 11, wherein the codon for the acylated tRNA having Formula I(a) or the codon for the acylated tRNA having Formula II(a) is selected from a codon for threonine, a codon for isoleucine, a codon for alanine or a codon for methionine.

17. The method of claim 11, wherein the sequence defined polymer prepared from a monomer selected from 4-oxo-4-phenylbutanoic acid, 3-oxo-3-phenylpropanoic acid, 3-phenylpropiolic acid, 2-hydrazineyl-4-oxo-4-phenylbutanoic acid, (Z)-3-chloro-3-(4-hydrazineylphenyl)acrylic acid, 2-hydrazineyl-2-methyl-3-oxobutanoic acid, 4-(4-hydrazineylphenyl)-4-oxobutanoic acid, 3-amino-4-oxo-4-phenylbutanoic acid, 2-amino-4-oxo-4-phenylbutanoic acid, 4-(4-(methylthio)phenyl)-4-oxobutanoic acid, 4-oxopentanoic acid, 4-oxohexanoic acid, 3-oxobutanoic acid, 3-oxopentanoic acid, 3-oxo-3-phenylpropanoic acid,5 -oxohexanoic acid, with a leaving group of either cyanomethylester (CME), dinitrobenzylester (DNB), or amino-derivatized benzyl thioester (ABT), as well as the synthesis of enantiomerically pure (L- or D-) and racemic aminophenylalanine, aminoglycine, amionalanine, aminovaline, aminoisoleucine, aminotyrosine with a leaving group of CME, DNB, and ABT.

18. A method for preparing a compound or molecule having a Formula III: 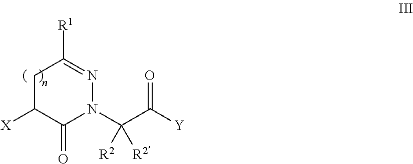
wherein:
X is hydrogen or the C-terminus of a polymer chain (e.g., the C-terminus of a polypeptide chain);
n is 0-8, 0-7, 0-6, 0-5, 0-4, 0-3, or 0-2;
R¹ is hydrogen, alkyl (e.g., methyl, ethyl), aryl (e.g., phenyl) which optionally is substituted at one or more positions with alkyl or alkylthio (e.g., 4-methylthio-phenyl), or le is the C-terminus of a peptide chain;
R² is hydrogen, alkyl (e.g., methyl, isopropyl), alkylaryl (e.g., benzyl) which optionally is substituted at one or more positions with hydroxyl (e.g., 3,4-dihydroxy-benzyl), or R² is the side chain of an amino acid (e.g., a side chain of an amino acid selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine;
R^{2′} is hydrogen or alkyl (e.g., methyl);
Y is hydrogen or the N-terminus of a polymer chain (e.g., the N-terminus of a polypeptide chain) or Y has a formula selected from —O(tRNA), —O(R³), or —NH(R³), wherein R³ is selected from hydrogen and alkyl;
the method comprising conjugating in a translation reaction the chemical moiety of an acylated tRNA having Formula I(a) and the chemical moiety of an acylated tRNA having Formula II(a): 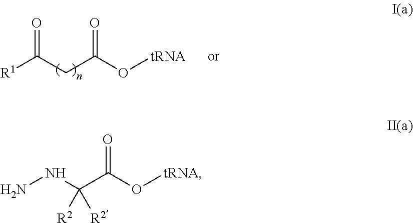
thereby forming the compound or molecule having Formula III.

19. A method for preparing an acylated tRNA molecule having a formula defined as: 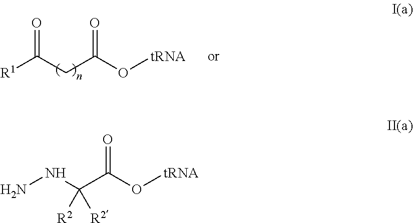
wherein:
n is 1-10, 1-9, 1-8, 1-7, 1-6, 1-5, 1-4, or 1-3;
R¹ is hydrogen, alkyl (e.g., methyl, ethyl), aryl (e.g., phenyl) which optionally is substituted at one or more positions with alkyl or alkylthio (e.g., 4-methylthio-phenyl),
R² is hydrogen, alkyl (e.g., methyl, isopropyl), alkylaryl (e.g., benzyl) which optionally is substituted at one or more positions with hydroxyl (e.g., 3,4-dihydroxy-benzyl), or R2 is the side chain of an amino acid (e.g., a side chain of an amino acid selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine; and
R^{2′} is hydrogen or alkyl (e.g., methyl);
the method comprising reacting in a reaction mixture:
(i) a flexizyme (Fx):
(ii) the tRNA molecule; and
(iii) a donor molecule having a formula: 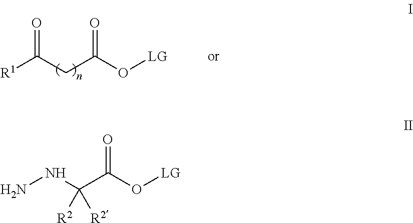
wherein:
LG is a leaving group that is removed when the chemical moiety is utilized to acylate a tRNA molecule (e.g., when the chemical moiety is utilized to acylate a tRNA molecule at the C3 hydroxyl group) and form an acylated tRNA having a Formula I(a) or II(a) and the Fx catalyzes an acylation reaction between the 3′ terminal ribonucleotide of the tRNA and the donor molecule to prepare the acylated tRNA molecule.

20. The method of claim 19, wherein the Fx is selected from aFx, dFx, and eFx.

21. The method of claim 19, wherein LG comprises a cyanomethyl moiety and the donor molecule comprises a cyanomethylester (CME).

22. The method of claim 19, wherein LG comprises a dinitrobenzyl moiety and the donor molecule comprises a dinitrobenzylester (DNB).

23. The method of claim 19, wherein LG comprises a (2-aminoethyl)amidocarboxybenzyl moiety and the donor molecule comprises a (2-aminoethyl)amidocarboxybenzyl thioester (ABT).

24. The method of claim 19, wherein the method is performed under reaction conditions such that at least about 5%, 10%, 15%, 20%, 25%, 30%, 35%, 40%, 45%, or 50% of the tRNA in the reaction mixture is acylated after reacting the reaction mixture for 120 hours, and preferably at least about 5%, 10%, 15%, 20%, 25%, 30%, 35%, 40%, 45%, or 50% of the tRNA in the reaction mixture is acylated after reacting the reaction mixture for 16 hours.


SEQUENCE LISTING (21 sequences)

SEQ ID NO: 1 - RNA, 22 nt, Artificial Sequence
  Synthetic sequence
ggcucuguucgcagagccgcca

SEQ ID NO: 2 - DNA, 33 bp, Artificial Sequence
  Synthetic- pJL1_StrepII_1
atgacctggtctcatccgcagttcgaaaaatag

SEQ ID NO: 3 - protein, 10 aa, Artificial Sequence
  Synthetic- strep tag
  misc_feature (1)..(1): Xaa can be any naturally occurring amino acid
XYWHSPQFEK

SEQ ID NO: 4 - protein, 12 aa, Artificial Sequence
  Synthetic peptide
MWHSPQFEKSAB

SEQ ID NO: 5 - protein, 13 aa, Artificial Sequence
  Synthetic peptide
MWHSPQFEKSABA

SEQ ID NO: 6 - protein, 14 aa, Artificial Sequence
  Synthetic peptide
MWHSPQFEKSABAB

SEQ ID NO: 7 - protein, 8 aa, Artificial Sequence
  Synthetic- streptavidin tag
WSHPQFEK

SEQ ID NO: 8 - DNA, 24 bp, Artificial Sequence
  Synthetic oligonucleotide
tggtctcatccgcagttcgaaaaa

SEQ ID NO: 9 - DNA, 45 bp, Artificial Sequence
  Synthetic oligonucleotide
catatgtcctggtctcatccgcagttcgaaaaatagtaagtcgac

SEQ ID NO: 10 - protein, 10 aa, Artificial Sequence
  Synthetic peptide
MTWSHPQFEK

SEQ ID NO: 11 - DNA, 57 bp, Artificial Sequence
  Synthetic oligonucleotide
catatgtggtctcatccgcagttcgaaaaatccaccatcaccatctagtaagtcgac

SEQ ID NO: 12 - protein, 14 aa, Artificial Sequence
  Synthetic peptide
MWSHPQFEKSTITI

SEQ ID NO: 13 - DNA, 63 bp, Artificial Sequence
  Synthetic oligonucleotide
catatgtggtctcatccgcagttcgaaaaatccaccatcaccatcaccatctagtaagtcgac

SEQ ID NO: 14 - protein, 16 aa, Artificial Sequence
  Synthetic peptide
MWSHPQFEKSTITITI

SEQ ID NO: 15 - DNA, 95 bp, Artificial Sequence
  Synthetic oligonucleotide fMet
gtaatacgactcactataggcggggtggagcagcctggtagctcgtcgggctcataacccgaagatcgtcggttcaaatccggcccccgcaacca

SEQ ID NO: 16 - DNA, 95 bp, Artificial Sequence
  Synthetic oligonucleotide Pro1E2
gtaatacgactcactatagggtgattggcgcagcctggtagcgcacttcgttggtaacgaaggggtcaggggttcgaatcccctatcacccgcca

SEQ ID NO: 17 - protein, 10 aa, Artificial Sequence
  Synthetic peptide
  misc_feature (1)..(1): Xaa can be any naturally occurring amino acid
XYWHSPQFEK

SEQ ID NO: 18 - protein, 14 aa, Artificial Sequence
  Synthetic peptide
  misc_feature (11)..(11): Xaa can be any naturally occurring amino acid
  misc_feature (13)..(13): Xaa can be any naturally occurring amino acid
MWHSPQFEKSXYXY

SEQ ID NO: 19 - protein, 16 aa, Artificial Sequence
  Synthetic peptide
  misc_feature (11)..(11): Xaa can be any naturally occurring amino acid
  misc_feature (13)..(13): Xaa can be any naturally occurring amino acid
  misc_feature (15)..(15): Xaa can be any naturally occurring amino acid
MWHSPQFEKSXYXYXY

SEQ ID NO: 20 - protein, 8 aa, Artificial Sequence
  Synthetic peptide
WHSPQFEK

SEQ ID NO: 21 - DNA, 1790 bp, Artificial Sequence
  plasmid pJL1_StrepII
ggatcctgcagttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaacgaattcagatctcgatcccgcgaaattaatacgactcactatagggagaccacaacggtttccctctagaaataattttgtttaactttaagaaggagatatacatatgtggtctcatccgcagttcgaaaaatagtaagtcgaccggctgctaacaaagcccgaaaggaagctgagttggctgctgccaccgctgagcaataactagcataaccccttggggcctctaaacgggtcttgaggggttttttgctgaaagccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgt